SEQUENCE LISTING (19 sequences)

SEQ ID NO: 1 - protein, 6 aa, Artificial Sequence
  Description of Artificial Sequence Synthetic peptide
CGRGDS

SEQ ID NO: 2 - protein, 6 aa, Artificial Sequence
  Description of Artificial Sequence Synthetic peptide
CGRGES

SEQ ID NO: 3 - protein, 12 aa, Artificial Sequence
  Description of Artificial Sequence Synthetic peptide
CGPQGIAGQGCR

SEQ ID NO: 4 - protein, 12 aa, Artificial Sequence
  Description of Artificial Sequence Synthetic peptide
CGPQGPAGQGCR

SEQ ID NO: 5 - protein, 7 aa, Artificial Sequence
  Description of Artificial Sequence Synthetic peptide
MNYYSNS

SEQ ID NO: 6 - protein, 7 aa, Artificial Sequence
  Description of Artificial Sequence Synthetic peptide
CNYYSNS

SEQ ID NO: 7 - protein, 4 aa, Artificial Sequence
  Description of Artificial Sequence Synthetic peptide
DAPS

SEQ ID NO: 8 - protein, 6 aa, Artificial Sequence
  Description of Artificial Sequence Synthetic peptide
AELDVP

SEQ ID NO: 9 - protein, 6 aa, Artificial Sequence
  Description of Artificial Sequence Synthetic peptide
VALDEP

SEQ ID NO: 10 - protein, 6 aa, Artificial Sequence
  Description of Artificial Sequence Synthetic peptide
  MOD_RES (3)..(3): 4-Hydroxyproline
GFPGER

SEQ ID NO: 11 - protein, 6 aa, Artificial Sequence
  Description of Artificial Sequence Synthetic peptide
NGRAHA

SEQ ID NO: 12 - protein, 8 aa, Artificial Sequence
  Description of Artificial Sequence Synthetic peptide
GPQGIAGQ

SEQ ID NO: 13 - protein, 8 aa, Artificial Sequence
  Description of Artificial Sequence Synthetic peptide
GPQGIWGQ

SEQ ID NO: 14 - protein, 8 aa, Artificial Sequence
  Description of Artificial Sequence Synthetic peptide
VPMSMRGG

SEQ ID NO: 15 - protein, 7 aa, Artificial Sequence
  Description of Artificial Sequence Synthetic peptide
QPQGLAK

SEQ ID NO: 16 - protein, 9 aa, Artificial Sequence
  Description of Artificial Sequence Synthetic peptide
GPLGLSLGK

SEQ ID NO: 17 - protein, 8 aa, Artificial Sequence
  Description of Artificial Sequence Synthetic peptide
GPLGMHGK

SEQ ID NO: 18 - protein, 4 aa, Artificial Sequence
  Description of Artificial Sequence Synthetic peptide
RGDS

SEQ ID NO: 19 - protein, 4 aa, Artificial Sequence
  Description of Artificial Sequence Synthetic peptide
RGES

(19) United States
(12) Patent Application Publication
(10) Pub. No.: US 2022/0143272 A1
(43) Pub. Date: May 12, 2022

(54) SYNTHETIC HEPARIN MIMETICS AND USES THEREOF

(71) Applicants: Massachusetts Institute of Technology, Cambridge, MA (US); Trustees of Boston University, Boston, MA (US)

(72) Inventors: Christopher S. Chen, Newton, MA (US); Sangeeta N. Bhatia, Lexington, MA (US); Linqing Li, Boston, MA (US); Jinling Yang, Boston, MA (US); Jeroen Eyckmans, Boston, MA (US)

(21) Appl. No.: 17/376,138

(22) Filed: Jul. 14, 2021

Related U.S. Application Data
(60) Provisional application No. 63/051,857, filed on Jul. 14, 2020.

(51) Int. Cl. A61L 27/50; A61L 27/20; C08B 37/02; A61L 27/54

(52) U.S. Cl. CPC A61L 27/507; A61L 27/20; A61L 2300/414; A61L 27/54; A61L 2430/36; C08B 37/0021

(57) ABSTRACT

Synthetic polymers, e.g., synthetic heparin mimetics, are provided, including hydrogel compositions incorporating the synthetic polymers. Methods of making and using the synthetic polymers are also provided.

### CROSS-REFERENCE TO RELATED APPLICATIONS

This application claims the benefit of U.S. Provisional Patent Application Ser. No. 63/051,857, filed Jul. 14, 2020, the entire contents of which are incorporated herein by reference.

### GOVERNMENT FUNDING

This invention was made with government support under Grant Nos. R01EB000262 and R01EB008396 awarded by the National Institute of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jul. 14, 2021, is named 127299-01902_SL.txt and is 4,359 bytes in size.

### BACKGROUND

A major challenge in tissue engineering is the development of synthetic biomaterials that induce and maintain functional vascularization of engineered tissue constructs after implantation. Establishing a functional vasculature that supplies sufficient oxygen and nutrient exchange is critical for the maintenance of tissue function as well as the survival and integration of engineered constructs after implantation, which remains one of the most fundamental challenges in regenerative medicine.

Many biomaterials derived from natural and synthetic sources, such as fibrin, collagen, hyaluronic acid, polyethylene glycol, alginate and chitosan, have been widely used in tissue engineering constructs as a starting point to support angiogenesis upon implantation. While none of these materials alone is sufficient to drive robust angiogenesis, infusing high concentrations of angiogenic growth factors leads to increased vascularization, but the effect is short-lived because of the rapid clearance of these diffusible factors out of the biomaterial. Accordingly, novel materials and strategies are needed to allow for the successful implantation and maintenance of engineered constructs.

### SUMMARY OF THE DISCLOSURE

In one aspect, the present invention provides a synthetic polymer comprising a polysaccharide comprising hydroxyl groups; wherein one or more of said hydroxyl groups has been modified by converting the hydroxyl groups into negatively charged functional groups, wherein said negatively charged functional groups provide an amount of negative charge to the synthetic polymer that is sufficient to promote one or more of binding of growth factors, growth factor activity, and vascularization.

In some embodiments, said synthetic polymer promotes one or more of binding of growth factors, growth factor activity and vascularization to a greater extent than a corresponding polysaccharide that has not been modified by converting the hydroxyl groups into negatively charged functional groups.

In some embodiments, said synthetic polymer does not impair blood coagulation.

In some embodiments, said synthetic polymer promotes greater binding of one or more growth factors as compared to a corresponding polysaccharide that has not been modified by converting the hydroxyl groups into negatively charged functional groups.

In some embodiments, said one or more growth factors is selected from the group consisting of a vascular endothelial growth factor (VEGF), a fibroblast growth factor (FGF), a bone morphogenic protein (BMP), an epidermal growth factor (EGF), a platelet derived growth factor (PDGF), a WNT, and a combination thereof.

In some embodiments, said one or more growth factors is a cytokine, optionally wherein the cytokine is an interleukin, an interferon, or chemokine.

In some embodiments, said synthetic polymer promotes greater growth factor activity as compared to a corresponding polysaccharide that has not been modified by converting the hydroxyl groups into negatively charged functional groups.

In some embodiments, the growth factor activity comprises growth factor-dependent cell signaling.

In some embodiments, said synthetic polymer promotes an equivalent or greater amount of growth factor dependent cell signaling as heparin.

In some embodiments, said synthetic polymer promotes vascularization.

In some embodiments, said synthetic polymer promotes greater vascularization as compared to a corresponding polysaccharide that has not been modified by converting the hydroxyl groups into negatively charged functional groups.

In some embodiments, said synthetic polymer is characterized by a zeta potential of −10 mV or less.

In another aspect, the present disclosure provides a synthetic polymer comprising a polysaccharide comprising hydroxyl groups; wherein one or more of said hydroxyl groups has been modified by converting the hydroxyl groups into negatively charged functional groups, wherein said synthetic polymer is characterized by a zeta potential of −10 mV or less.

In some embodiments, said synthetic polymer is characterized by a zeta potential of about −10 mV to about −60 mV.

In some embodiments, said synthetic polymer is characterized by a zeta potential of about −10 mV to about −30 mV, about −20 mV to about −40 mV, about −30 mV to about −50 mV, about −40 mV to about −60 mV, or about −50 mV to about −60 mV.

In some embodiments, said synthetic polymer comprises an average of 0.1 to 2.0 negatively charged functional groups per monosaccharide unit. In some embodiments, said synthetic polymer comprises an average of 0.5 to 1.5 negatively charged functional groups per monosaccharide unit. In some embodiments, said synthetic polymer comprises an average of at least 0.5 negatively charged functional groups per monosaccharide unit.

In some embodiments, said polysaccharide is a naturally occurring polysaccharide.

In some embodiments, said naturally occurring polysaccharide is selected from the group consisting of alginate, agarose, chondroitin sulfate, chitin/chitosan, cellulose, dextran, starch, glycogen, galactogen, inulin, pectin, and hyaluronic acid.

In some embodiments, said polysaccharide comprises repeating monosaccharide units prior to modification of the hydroxyl groups.

In some embodiments, said polysaccharide comprises repeating disaccharide units prior to modification of the hydroxyl groups.

In some embodiments, said polysaccharide comprises repeating polysaccharide units prior to modification of the hydroxyl groups.

In some embodiments, said polysaccharide is dextran.

In some embodiments, said negatively charged functional groups are selected from the group consisting of a sulfate group, a phosphate group, a carboxyl group and combinations thereof.

In some embodiments, said synthetic polymer has a mean weight-average molecular weight of about 5 kDa to about 650 kDa.

In some embodiments, said synthetic polymer has a mean weight-average molecular weight of about 50 to about 100 kDa.

In some embodiments, said synthetic polymer has a mean weight-average molecular weight of about 70 kDa to about 90 kDa.

In some embodiments, said synthetic polymer is characterized by a zeta potential of about −20 mV to about −30 mV.

In some embodiments, said synthetic polymer has a mean weight-average molecular weight of about 450 kDa to about 650 kDa.

In some embodiments, said synthetic polymer is characterized by a zeta potential of about −40 mV to about −50 mV.

In another aspect, the disclosure provides a synthetic dextran polymer having one or more hydroxyl groups naturally present in dextran modified by converting the hydroxyl groups into negatively charged functional groups, wherein said synthetic dextran polymer has a zeta potential of about −20 mV to about −50 mV.

In some embodiments, the synthetic dextran polymer comprises an average of at least 0.5 negatively charged functional groups per monosaccharide unit of the polymer.

In some embodiments, said negatively charged functional groups are sulfate groups.

In some embodiments, the synthetic polymer has a mean weight-average molecular weight of about 10 kDa to about 650 kDa, about 30 kDa to about 50 kDa, about 70 kDa to about 80 kDa, or about 450 kDa to about 650 kDa.

In one aspect, the present disclosure provides a method for generating the synthetic polymer of the disclosure, said method comprising contacting a polysaccharide comprising hydroxyl groups with a moiety comprising a negatively charged functional group under conditions that allow for conversion of one or more of said hydroxyl groups into negatively charged functional groups.

In one aspect, the present disclosure provides a hydrogel comprising a plurality of the synthetic polymers, wherein the synthetic polymers are cross-linked to each other by a cross-linker.

In another aspect, the present disclosure provides a hydrogel comprising a plurality of synthetic polymers cross-linked to each other by a cross-linker, wherein each of said synthetic polymers comprises a polysaccharide comprising hydroxyl groups; wherein one or more of said hydroxyl groups has been modified by converting the hydroxyl groups into negatively charged functional groups.

In another aspect, the present disclosure provides hydrogel comprising a plurality of synthetic dextran polymers cross-linked to each other by a cross-linker, wherein each of said synthetic dextran polymers comprises a dextran polymer in which one or more hydroxyl groups naturally present in dextran has been modified by converting the hydroxyl groups into negatively charged functional groups.

In some embodiments, the negatively charged functional groups provide an amount of negative charge to the synthetic polymer that is sufficient to promote one or more binding of growth factors, growth factor activity, and vascularization, or wherein the synthetic polymer has a zeta potential of about −10 mV to about −60 mV.

In some embodiments, said cross-linker is a non-covalent cross-linker.

In some embodiments, said cross-linker is an ionic cross-linker.

In some embodiments, said cross-linker is a covalent cross-linker.

In some embodiments, said cross-linker is a peptide cross-linker.

In some embodiments, said cross-linker is a cleavable cross-linker.

In some embodiments, said cleavable cross-linker is a matrix metalloproteinase (MMP)-cleavable peptide.

In some embodiments, said peptide comprises an amino acid sequence CGPQGIAGQGCR (SEQ ID NO: 3).

In some embodiments, said synthetic polymers further comprised alkene containing moieties covalently attached to the polysaccharide polymer chains prior to cross-linking.

In some embodiments, the alkene containing moiety is methacrylate, acrylate, or maleimide.

In some embodiments, the hydrogel further comprises a cell-adhesive peptide.

In some embodiments, the cell-adhesive peptide comprises an amino acid sequence RGD.

In some embodiments, said cell-adhesive peptide comprises an amino acid sequence CGRGDS (SEQ ID NO: 1).

In some embodiments, the hydrogel further comprises at least one growth factor.

In some embodiments, said at least one growth factor is a selected from the group consisting of a vascular endothelial growth factor (VEGF), a fibroblast growth factor (FGF), a bone morphogenic protein (BMP), an epidermal growth factor (EGF), a platelet derived growth factor (PDGF), a WNT, and a combination thereof.

In some embodiments, said at least one growth factor is a cytokine, optionally wherein the cytokine is an interleukin, an interferon, or chemokine.

In some embodiments, the hydrogel further comprises a population of cells.

In some embodiments, said population of cells comprises one cell type.

In some embodiments, said population of cells comprises two or more cell types.

In some embodiments, said population of cells comprises parenchymal cells.

In some embodiments, said parenchymal cells are of heart, lung, liver, kidney, adrenal gland, pituitary gland, pancreas, or muscle.

In some embodiments, said population of cells comprises stromal cells.

In some embodiments, said population of cells comprises endothelial cells.

In some embodiments, said population of cells comprises endothelial cells and fibroblasts.

In one aspect, the disclosure provides a composition comprising the synthetic polymer or the hydrogel as described herein.

In some embodiments, the composition further comprises a growth factor.

In one aspect, the disclosure provides a method of promoting vascularization of a cell implant or an engineered tissue construct in a subject, comprising administering to the subject the cell implant or engineered tissue construct in combination with the synthetic polymer, the hydrogel, or the composition described herein.

In some embodiments, promoting vascularization of a cell implant or an engineered tissue construct results in an amount of vascularization of an engineered tissue construct that is greater than the amount of vascularization of an engineered tissue construct obtained using a corresponding polysaccharide in which hydroxyl groups have not been modified by converting the hydroxyl groups into negatively charged functional groups or using a hydrogel comprising said corresponding polysaccharide.

In another aspect, the disclosure provides a method of promoting cell survival in a cell implant or an engineered tissue construct in a subject, comprising administering to the subject the cell implant or engineered tissue construct in combination with the synthetic polymer, the hydrogel, or the composition described herein.

In some embodiments, promoting cell survival in a cell implant or an engineered tissue construct results in a greater cell survival in a cell implant or an engineered tissue construct than cell survival in a cell implant or an engineered tissue construct achieved using a corresponding polysaccharide in which hydroxyl groups have not been modified by converting the hydroxyl groups into negatively charged functional groups or using a hydrogel comprising said corresponding polysaccharide.

In another aspect, the disclosure provides a method of promoting engraftment of a cell implant or an engineered tissue construct in a subject, comprising administering to the subject the cell implant or engineered tissue construct in combination with the synthetic polymer, the hydrogel, or the composition of claims described herein.

In some embodiments, promoting engraftment of a cell implant or an engineered tissue construct results in a greater engraftment of a cell implant or an engineered tissue construct than engraftment of a cell implant or an engineered tissue construct achieved using a corresponding polysaccharide in which hydroxyl groups have not been modified by converting the hydroxyl groups into negatively charged functional groups or using a hydrogel comprising said corresponding polysaccharide.

In another aspect, the disclosure provides a method of promoting vascularization in a diseased or damaged tissue in a subject, comprising administering to the subject the synthetic polymer, the hydrogel, or the composition described herein.

In some embodiments, promoting vascularization in a diseased tissue results in a higher vascularization in a diseased tissue than vascularization in a diseased tissue achieved using a corresponding polysaccharide in which hydroxyl groups have not been modified by converting the hydroxyl groups into negatively charged functional groups or using a hydrogel comprising said corresponding polysaccharide.

In some embodiments, said diseased tissue comprises a region of ischemia.

In one aspect, the disclosure provides a method of promoting vascularization of a tissue graft in a subject, comprising contacting a tissue to be grafted with the synthetic polymer, the hydrogel, or the composition of claims described herein prior to grafting of the tissue for a sufficient time to promote vascularization of the tissue graft upon grafting in the subject.

In another aspect, the disclosure provides a method of promoting a growth factor-dependent cell therapy, comprising administering to a subject the growth factor-dependent cell therapy in combination with the synthetic polymer, the hydrogel, or the composition described herein, such that the growth factor-dependent cell therapy is promoted.

In another aspect, the disclosure provides a method of promoting activity of a growth factor in a subject, comprising administering to a subject the growth factor in combination with the synthetic polymer, the hydrogel, or the composition described herein, such that the growth factor activity is promoted.

### Other Embodiments

In some aspects, the disclosure provides a synthetic polymer, e.g., synthetic heparin mimetic, comprising a polymeric carbohydrate backbone of repeating polysaccharide units, each unit having one or more chemically reactive hydroxyl groups, wherein the mimetic is modified at the one or more hydroxyl groups with a functional group to provide a negative charge to the mimetic to promote growth factor binding and/or growth factor activity. In some aspects, the repeating polysaccharide units are the same. In some aspects, the synthetic polymer, e.g., synthetic heparin mimetic, is a homopolymer. In other aspects, the repeating polysaccharide units comprise 2, 3 or more different polysaccharide units.

In any of the foregoing or related aspects, the functional group is selected from a sulfate group, a phosphate group, a carboxylic group, other negatively charged moieties, and mixtures thereof. In some aspects, the functional group is a sulfate group.

In any of the foregoing or related aspects, the synthetic polymer, e.g., synthetic heparin mimetic, comprises a zeta potential of −10 to −50 millivolts (mV). In some aspects, the synthetic polymer, e.g., synthetic heparin mimetic, comprises a zeta potential of −10 to −20, −10 to −30, −10 to −40, −15 to −25, −15 to −35, −15 to −45, −15 to −55, −20 to −30, −20 to −40, −20 to −50, −20 to −60, −25 to −35, −25 to −45, −25 to −55, −25 to −65, −30 to −40, −30 to −50, −30 to −60, −30 to −70, −35 to −45, −35 to −55, −35 to −65, −35 to −75, −40 to −50, −40 to −60, −40 to −70, −40 to −80, −45 to −55, −45 to −65, −45 to −75, or −45 to −85 mV.

In any of the foregoing or related aspects, the synthetic polymer, e.g., synthetic heparin mimetic, has a mean weight-average molecular weight of 70 to 90 kDa. In some aspects, the synthetic polymer, e.g., synthetic heparin mimetic, has a mean weight-average molecular weight of 5 to 30 kDa, 10 to 30 kDa, 50 to 70 kDa, 50 to 90 kDa, 70 to 90 kDa, 90 to 110 kDa, or 150-500 kDa. In some aspects, the synthetic polymer, e.g., synthetic heparin mimetic, has a mean weight-average molecular weight of less than 70 kDa. In some aspects, the synthetic polymer, e.g., synthetic heparin mimetic, has a mean weight-average molecular weight of at least 90 kDa.

In any of the foregoing or related aspects, each repeating polysaccharide unit of the synthetic polymer, e.g., synthetic heparin mimetic, comprises 0.5-2.0 functional groups per repeating unit. In some aspects, each repeating polysaccharide unit of the synthetic polymer, e.g., synthetic heparin mimetic, comprises 0.5-1.0, 1.0-2.0, 0.5-3.0, 1.0-3.0, or 2.0-3.0 functional groups per repeating unit.

In any of the foregoing or related aspects, the growth factor is a VEGF, FGF, or combination thereof. In some aspects, the growth factor is selected from the group of angiopoietins, extracellular matrix proteins, adhesion proteins, BMPs, TGFbeta, SDFs, interleukins, interferons, CXCLs, lipoproteins or other polypeptides that bind and activate cellular receptors. In some aspects, the growth factor is any polypeptide having a positive charge.

In any of the foregoing or related aspects, the synthetic polymer, e.g., synthetic heparin mimetic, has reduced anti-coagulant activity relative to heparin. In some aspects, the anti-coagulant activity is reduced by 10%, 20%, 30%, 40%, 50%, 60%, 70%, 80%, 90%, or 100%. In some aspects, the anti-coagulant activity is reduced by at least 50%.

In any of the foregoing or related aspects, the synthetic polymer, e.g., synthetic heparin mimetic, comprises a polysaccharide selected from the group of alginate, agarose, chondroitin sulfate, chitin/chitosan, cellulose, starch, and glycogen. In some aspects, the synthetic polymer, e.g., synthetic heparin mimetic, comprises dextran.

In some aspects, the disclosure provides a synthetic polymer, e.g., synthetic heparin mimetic, comprising a polymeric carbohydrate backbone of repeating polysaccharide units, each unit having one or more chemically reactive hydroxyl groups, wherein the mimetic polymer is modified at the one or more hydroxyl groups with a functional group to provide a negative charge to the mimetic to promote growth factor binding and/or growth factor activity.

In some aspects, the synthetic polymer, e.g., synthetic heparin mimetic, comprises dextran as the polysaccharide. Dextran is represented by the following structure in schematic A having 3 reactive hydroxyl groups (“C2, C3 and C4”) on each monosaccharide unit, wherein n is 100-1000, 200-800, 300-600 or 400-500 (repeating units), Due to the structure and space availability, the reactive preference of —OH (hydroxyl) is C2>C4>C3:

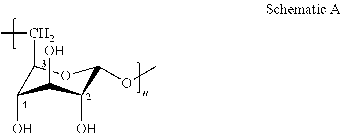

Accordingly, in some aspects, the disclosure provides a synthetic polymer, e.g., a heparin mimetic, that is a syntheticdextran polymer. The monosaccharide unit of dextran has 3 reactive hydroxyl groups on 3 carbons atoms (“C2, C3 and C4”), as shown in schematic A. In some embodiments, the synthetic dextran polymer comprises monosaccharides in which one, two, or three of the reactive hydroxyl groups on C2, C3 and C4 of each unit are modified and converted into a functional group independently selected from a sulfate group, a phosphate group, a carboxylic group, other negatively charged moieties, and mixtures thereof. In some embodiments, the synthetic dextran polymer may comprise a mixture of unmodified monosaccharides and modified monosaccharides, e.g., monosaccharides in which the hydroxyl groups on carbons C2 and/or C3 and/or C4 have been converted into negatively charged functional groups.

In other aspects, the disclosure provides a method for generating a synthetic polymer, e.g., synthetic heparin mimetic, as described herein, comprising contacting the polymeric carbohydrate backbone with the functional group under conditions that allow for a chemical reaction between the hydroxyl group and the functional group.

In yet other aspects, the disclosure provides a composition comprising a modified dextran molecule having at least one chemically reactive hydroxyl group modified with a sulfate group to provide a negative charge, wherein the modified dextran molecule has a mean weight-average molecular weight of 70-90 kDa and a zeta potential of −20 to −30 mV. In other aspects, the disclosure provides a composition comprising a modified dextran molecule having at least one chemically reactive hydroxyl group modified with a sulfate group to provide a negative charge, wherein the modified dextran molecule has a mean weight-average molecular weight of 30-150 kDa and a zeta potential of −10 to −50 mV.

In any of the foregoing or related aspects, the dextran molecule comprises repeating polysaccharide units, each unit comprising 0.5-2.0 sulfate groups per repeating unit. In some aspects, each unit comprises 0.5-1.0, 1.0-2.0, 0.5-3.0, 1.0-3.0, or 2.0-3.0 sulfate groups per repeating unit.

In any of the foregoing or related aspects, the negative charge of the modified dextran molecule promotes growth factor binding and/or growth factor activity. In some aspects, the growth factor is a VEGF, FGF, or combination thereof. In some aspects, the growth factor is selected from the group of angiopoietins, extracellular matrix proteins, adhesion proteins, BMPs, TGFbeta, SDFs, interleukins, interferons, CXCLs and lipoproteins. In some aspects, the growth factor is any polypeptide having a positive charge.

In any of the foregoing or related aspects, the modified dextran molecule has reduced anti-coagulant activity relative to heparin. In some aspects, the anti-coagulant activity is reduced by 10%, 20%, 30%, 40%, 50%, 60%, 70%, 80%, 90%, or 100%. In some aspects, the anti-coagulant activity is reduced by at least 50%.

In other aspects, the disclosure provides a hydrogel comprising a plurality of synthetic polymers, e.g., synthetic heparin mimetics, described herein, wherein the synthetic polymers, e.g., synthetic heparin mimetics, are cross-linked via a cross-linker. In yet other aspects, the disclosure provides a hydrogel comprising modified dextran molecules each having at least one chemically reactive hydroxyl group modified with a sulfate group to provide a negative charge, wherein the modified dextran molecules are cross-linked via a cross-linker.

In any of the foregoing or related aspects, the cross-linker in the hydrogel is a cleavable cross-linker. In some aspects, the cleavable cross-linker is a matrix metalloproteinase (MMP)-cleavable dithiol-containing crosslinker peptide. In some aspects, the crosslinker peptide is CGPQGIAGQGCR (SEQ ID NO: 3).

In any of the foregoing or related aspects, the synthetic polymer, e.g., synthetic heparin mimetic, or the modified dextran molecule is functionalized with methacrylate prior to cross-linking.

In any of the foregoing or related aspects, the hydrogel further comprises a cell-adhesive peptide. In some aspects, the cell-adhesive peptide is an extracellular matrix-derived adhesive peptide. In some aspects, the cell-adhesive peptide is a collagen-derived adhesive peptide. In some aspects, the cell-adhesive peptide is a laminin-derived adhesive peptide. In some aspects, the cell-adhesive peptide is a fibronectin-derived adhesive peptide. In some aspects, the cell-adhesive peptide is CGRGDS (SEQ ID NO: 1).

In any of the foregoing or related aspects, the hydrogel further comprises at least one growth factor. In some aspects, the growth factor is a VEGF, FGF, or combination thereof. In some aspects, the growth factor is selected from the group of angiopoietins, extracellular matrix proteins, adhesion proteins, BMPs, TGFbeta, SDFs, interleukins, interferons, CXCLs and lipoproteins. In some aspects, the growth factor is any polypeptide having a positive charge.

In any of the foregoing or related aspects, the hydrogel further comprises at least one population of cells. In other aspects, the hydrogel further comprises at least two different populations of cells.

In any of the foregoing or related aspects, cells within the hydrogel are capable of forming multicellular sprouts, and the number of multicellular sprouts is increased relative to a hydrogel without the heparin mimetic or modified dextran molecule.

In other aspects, the disclosure provides a method of increasing vascularization of an engineered tissue construct in a subject, comprising administering to the subject the engineered tissue construct in combination with a synthetic polymer, e.g., synthetic heparin mimetic, a modified dextran molecule, a composition, or a hydrogel, described herein, wherein vascularization is increased relative to an engineered tissue construct administered without the heparin mimetic, the composition, or the hydrogel.

In other aspects, the disclosure provides a method of increasing survival of an engineered tissue construct in a subject, comprising administering to the subject the engineered tissue construct in combination with a synthetic polymer, e.g., synthetic heparin mimetic, a modified dextran molecule, a composition, or a hydrogel, described herein, wherein survival is increased relative to an engineered tissue construct administered without the heparin mimetic, the composition, or the hydrogel.

In other aspects, the disclosure provides a method of increasing engraftment of an engineered tissue construct in a subject, comprising administering to the subject the engineered tissue construct in combination a synthetic polymer, e.g., synthetic heparin mimetic, a modified dextran molecule, a composition, or a hydrogel, described herein, wherein engraftment is increased relative to an engineered tissue construct administered without the heparin mimetic, the composition, or the hydrogel.

In yet other aspects, the disclosure provides a method of promoting angiogenesis in a diseased tissue in a subject, comprising administering to the subject a synthetic polymer, e.g., synthetic heparin mimetic, a modified dextran molecule, a composition, or a hydrogel, described herein. In some aspects, the diseased tissue comprises a region of ischemia.

In other aspects, the disclosure provides a kit comprising a synthetic polymer, e.g., synthetic heparin mimetic, described herein and instructions for administering the mimetic with an engineered tissue construct to improve the survival, vascularization and/or engraftment of the construct. In some aspects, the instructions comprise administration of the mimetic simultaneously or sequentially with the construct.

In further aspects, the disclosure provides a kit comprising a synthetic polymer, e.g., synthetic heparin mimetic, described herein and instructions for promoting angiogenesis in a diseased tissue in a subject by administering the mimetic to a subject having a diseased tissue.

In other aspects, the disclosure provides a kit comprising a modified dextran molecule described herein and instructions for administering the molecule with an engineered tissue construct to improve the survival, vascularization and/or engraftment of the construct. In some aspects, the instructions comprise administration of the molecule simultaneously or sequentially with the construct.

In further aspects, the disclosure provides a kit comprising a modified dextran molecule described herein and instructions for promoting angiogenesis in a diseased tissue in a subject by administering the molecule to a subject having a diseased tissue.

In other aspects, the disclosure provides a kit comprising a hydrogel described herein and instructions for administering the hydrogel with an engineered tissue construct to improve the survival, vascularization and/or engraftment of the construct. In some aspects, the instructions comprise administration of the hydrogel simultaneously or sequentially with the construct.

In further aspects, the disclosure provides a kit comprising a hydrogel described herein and instructions for promoting angiogenesis in a diseased tissue in a subject by administering the hydrogel to a subject having a diseased tissue.

In other aspects, the disclosure provides a hydrogel comprising a plurality of modified dextran molecules conjugated with heparin, wherein each modified dextran molecule comprises repeating units comprising at least one chemically reactive hydroxyl group modified with a sulfate group to provide a negative charged, and wherein the dextran molecules are cross-linked via a cross-linker.

### BRIEF DESCRIPTION OF FIGURES

FIG. 1 is a schematic showing formulation of synthetic and pro-angiogenic hydrogels containing Dex-MA (dextran functionalized with methacrylate) co-crosslinked with either chemically conjugated heparin or soluble heparin in the presence of thiolated cell-adhesive peptide and di-thiol terminated MMP-cleavage peptide crosslinkers via Michael-type addition reaction at pH 8.

FIG. 2A is a graph showing tunable hydrogel stiffness through modulating bulk material solution concentrations (wt %) or crosslinking ratio (macromer:crosslinker), yielding hydrogels with stiffness ranging from 200 Pa (2 wt %, 1:1) to 4500 Pa (4 wt % 1:1).

FIG. 2B is a graph showing that the synthetic material system permits independent control of cell adhesion motif (CGRGDS (SEQ ID NO: 1)) and matrix degradation crosslinkers (degradable: CGPQGIAGQGCR (SEQ ID NO: 3) versus non-degradable: CGPQGPAGQGCR (SEQ ID NO: 4)) in dextran hydrogels while maintaining hydrogel stiffness consistent. Represented are measurement of hydrogel stiffness for dextran hydrogels (i.e., Dex-MA) crosslinked with either degradable or non-degradable crosslinker and formulated with the cell adhesion motif.

FIG. 2C is a graph showing in situ degradation of dextran hydrogels crosslinked with MMP-cleavable peptides designed to elicit different degradation kinetics. Dextran was crosslinked using the degradable crosslinker set forth by SEQ ID NO: 3 or the non-degradable cross-linker set forth by SEQ ID NO: 4 to generate a degradable dextran hydrogel (“HD”) or non-degradable dextran hydrogel (“SD”) respectively. Degradable and non-degradable dextran hydrogels were incubated with 0.2 mg/mL collagenase at 37° C. for 72 hours, and hydrogel weights were monitored. Degradable dextran hydrogels exhibited significant matrix degradation leading to significant decrease in hydrogel weights while slow-degradable gels or degradable gels incubated with PBS showed minimum weight loss.

FIG. 2D is a graph showing mechanical properties of hydrogels formulated with various material compositions and crosslinked with varying crosslinking densities to achieve different hydrogel stiffness. Dex-MA=hydrogel formed from dextran functionalized with methacrylate; sHep=hydrogel formed from Dex−MA and containing soluble heparin; cHep-MA=hydrogel formed from heparin-conjugated dextran and functionalized with methacrylate

FIG. 3 provides confocal images of encapsulated of human dermal fibroblasts in dextran-based hydrogels (i.e., Dex−MA) with various matrix stiffness (soft, intermediate, or stiff) and Dex-MA prepared without cell adhesive sequence (no RGD). Human dermal fibroblasts were encapsulated at 1×10⁶/mL and cultured in dextran hydrogels and fluorescent images were taken at day 3 following encapsulation.

FIG. 4 shows confocal images of in vitro vascular network formation (scale bar, 100 μm) through 3D co-culturing of Ruby-LifeAct-HUVECs (human umbilical vein endothelial cells) and GFP-HDFs (human dermal fibroblasts) in dextran-based biomimetic hydrogels with different material compositions; dextran gels without heparin (Dex-MA+GFs), dextran gels with soluble non-reactive heparin (sHep+GFs), dextran gels with conjugated heparin (cHep−MA+GFs) and dextran gels with conjugated heparin but without growth factors (cHep−MA). HUVECs and HDFs were encapsulated at 6 million/mL and 3 million/mL cell density in 4 wt % dextran hydrogels (dextran:heparin=90%:10% (w/w)) at an intermediate crosslinking density (1 to 0.75) to reach a stiffness approximately to 2000 Pa. Cell-laden hydrogels were cultured in regular EMG-2 medium with medium changes every 2 days, and samples were fixed after 14 days and formation of a vasculature network was imaged. Hydrogels also contained growth factors VEGF and bFGF (GFs) where indicated.

FIGS. 5A-5D are graphs showing quantitative analysis of vascular network structure cells cultured and imaged in FIG. 4. FIG. 5A shows vessel density quantification, defined by percentage of total endothelial cell area per image frame. Additional vascular network structure was analyzed via quantifying average vessel length (FIG. 5B), total vessel length (FIG. 5C), and number of branch points (FIG. 5D) per field of image with ****P<0.0001.

FIGS. 6A & 6B show HUVEC-aggregates in dextran hydrogels. FIG. 6A provides representative bright field images of multicellular HUVEC-aggregates encapsulated in dextran hydrogels engineered with different angiogenic features elicited different angiogenic sprouting behavior, scale bar: 100 μm. Comparison was made between dextran hydrogels loaded with VEGF and bFGF (Dex-MA+GFs), dextran hydrogel loaded with soluble heparin, VEGF, and bFGF (sHep+GFs), heparin-conjugated dextran hydrogel loaded with VEGF and bFGF (cHep−MA+GFs), and heparin-conjugated dextrin hydrogel without growth factors (cHep−MA) FIG. 6B shows the degree of angiogenesis quantified by comparing number of endothelial sprouts per aggregate in different hydrogel compositions. HUVEC aggregates were encapsulated ˜1000 aggregates/mL density and cultured in regular EGM-2 medium with medium changes every 2 days, and samples were fixed and imaged after 5 days, *P<0.05 and ****P<0.0001.

FIG. 7A shows representative images and a graph showing quantitative analysis of host blood vessels invading into different hydrogel compositions implanted into mice based on percentage of mCD31 positive area. Confocal imaging was performed on hydrogels harvested at day 14 post-implantation (n≥4) scale bar, 200 μm. mCD31 is a marker for mouse endothelial cells.

FIG. 7B shows representative images and a graph showing quantification of perfused host vessels quantified by FITC-dextran (70 kDa) positive area in different hydrogel compositions implanted into mice. Confocal imaging was performed on hydrogels harvested at day 14 post-implantation (n≥4) scale bar, 200 μm.

FIG. 7C shows representative images and a graph showing quantification of skin area showing local hemorrhage side effects (outlined in black in top panel, right-most image) induced by implantation of heparin-containing hydrogels (i.e., heparin-conjugated dextran hydrogel loaded with VEGF and bFGF growth factors) at day 1 relative to hydrogel without heparin (i.e., dextran hydrogel loaded with VEGF and bFGF growth factors).

FIG. 8 provides a fluorescent image of in vivo implantation of heparinized dextran gels containing human-hepatocyte aggregates (top) and a graph showing hepatic function of hydrogels having dextran-conjugated heparin and loaded with VEGF and bFGF (cHep+GFs) comprising human hepatocytes and implanted in mice as measured by human albumin secretion at days 5, 10 and 14 post-implantation (bottom).

FIG. 9A is a schematic of sulfated dextran hydrogel formation with increased negatively charge characteristics to mimic native heparin. Synthetic heparin-mimetic hydrogels were formulated via co-crosslinking Dex-MA and sulfated-Dex-MA in the presence of thiolated cell-adhesive peptide and di-thiol terminated MMP-cleavage peptide crosslinkers via Michael-type addition reaction at pH 8, identical crosslinking reaction employed in heparin-conjugated dextran gels. For exemplary hydrogels containing sulfated-dextrin, the sulfated dextran is highly sulfated dextran (HS-Dex-MA) or low sulfated dextran (LS-Dex-MA), and the hydrogels are formed by crosslinking methacrylated dextran (Dex-MA) and sulfated Dex-MA, e.g., at a ratio of Dex-MA to sulfated-Dex-MA of 80 to 20 (w/w (%)).

FIG. 9B provides schematics showing the chemical reaction from dextran to methacrylate dextran (top), dextran to sulfated dextran (middle), and methacrylated dextran to sulfated methacrylated dextran (bottom).

FIG. 9C is a graph showing the zeta potential of sulfated dextran at different degree of sulfation with comparison to unmodified dextran (Dex), methacrylated dextran (Dex-MA), native heparin and methacrylated heparin (Heparin-MA). The sulfated dextran was either highly sulfated dextran (HS-Dex-MA) or low sulfated dextran (LS-Dex-MA).

FIG. 9D is a graph showing hydrogel stiffness via oscillatory shear rheological cauterizations of various hydrogel compositions, showing no statistical differences among groups with shear modulus (G′˜2000 Pa). The hydrogels included dextran hydrogel (Dex-MA), heparin-conjugated dextran hydrogel (cHep-MA), dextran hydrogel loaded with soluble heparin (sHep), low sulfated dextran hydrogel (LS-Dex-MA), and high sulfated dextran hydrogel (HS-Dex-MA).

FIG. 9E is a graph showing swelling measurements of hydrogels made of various compositions as indicated in FIG. 9D.

FIG. 9F is a graph showing quantitative analysis of anticoagulant activity of heparin-MA, Dex-MA and sulfated-Dex-MA using a tail-bleeding assay.

FIG. 9G provides graphs showing zeta potential of sulfated dextran of different mean weight-average molecular weights (˜10 kDa, ˜40 kDa and ˜450-650 kDa) (top and bottom left panels) and zeta potential of heparin and methacrylated heparin from different sources (bottom right panel).

FIG. 9H shows NMR spectra of Dex, Dex-MA and HS-Dex-MA, showing that sulfation modification does not change methacrylation degree.

FIGS. 10A-10D show western blot and quantitative analysis to assess angiogenesis signaling pathways with HUVECs cultured on various hydrogel compositions. Western blot (FIG. 10A) and quantitative analysis of pVEGFR2 (FIG. 10B), pERK1/2 (FIG. 10C) and pAkt signaling (FIG. 10D). The hydrogels evaluated included dextran hydrogel loaded with VEGF and bFGF (Dex-MA+GFs), heparin-conjugated dextran hydrogel either loaded with VEGF and bFGF or containing no growth factors (cHep-MA+GFs and cHep-MA respectively), dextran hydrogel loaded with soluble heparin, VEGF and bFGF (sHep+GFs), low sulfated dextran hydrogel loaded with VEGF and bFGF (LS-Dex-MA+GFs), and high sulfated dextran hydrogel loaded with VEGF and bFGF (HS-Dex-MA+GFs).

FIG. 11A provides fluorescent images of various dextran-derived hydrogels supporting HUVEC cell attachment and proliferation in vitro. Dex-MA: dextran hydrogel; Dex-MA+GFs: dextran hydrogel loaded with VEGF and bFGF; cHep-MA+GFs: heparin-conjugated dextran hydrogel loaded with VEGF and bFGF; LS-Dex-MA+GFs and HS-Dex-MA+GFs: low and high sulfated dextran hydrogel loaded with VEGF and bFGF, respectively.

FIG. 11B provides fluorescent images of in vitro vascularization of human hepatocyte aggregates (top) and a graph showing albumin production by the human hepatocytes (bottom) in dextran hydrogel loaded with VEGF and bFGF (noHep+GFs), dextran hydrogel loaded with soluble heparin, VEGF and bFGF (sHep+GFs), heparin-conjugated dextran hydrogel loaded with VEGF and bFGF (cHep+GFs) and high sulfated dextran hydrogel loaded with VEGF and bFGF (sDex+GFs).

FIGS. 12A-12F provide representative bright field and confocal fluorescent images and quantification of in vitro vascular network formation through co-culturing of Ruby-Lifeact-HUVECs and GFP-HDFs in sulfated dextran hydrogels. FIG. 12A shows bright field and confocal images of cells in hydrogels (Dex-MA:sulfated-Dex-MA=80:20, w/w (%)), LS-Dex-MA (low sulfation dextran), HS-Dex-MA (high sulfation dextran)), scale bar, 100 μm. FIG. 12B shows representative bright field images of multicellular HUVEC-aggregates encapsulated in sulfated dextran hydrogels engineered with different sulfate degrees, scale bar: 200 μm. The degree of angiogenesis is quantified by comparing number of endothelial sprouts per aggregate in different hydrogel compositions. HUVEC-aggregates were encapsulated ˜1000 aggregates/mL density and cultured in regular EGM-2 medium with medium changes every 2 days, and samples were fixed and imaged after 5 days, ***P<0.001. FIG. 12C-F show quantitative assessment of in vitro vascular network structure at day 14, through quantifying vessel density (FIG. 12C), average vessel length (FIG. 12D), number of branch points (FIG. 12E); and quantification of in vitro angiogenic sprouting via counting multicellular endothelial sprouts (FIG. 12F), n≥4.

FIG. 12G provides fluorescent images of vascularization of sulfated-dextran hydrogels, (left image: GFP-HDF channel, middle image: Ruby-HUVEC channel, and right image: overlay). GFP-HDF and Ruby-HUVEC cells in vitro supported by sulfated dextran of ˜400-600 kDa mean weight-average molecular weight (˜550 kDa) and zeta potential of ˜−47 mV.

FIG. 13 shows orthogonal views of vascular network formed in highly sulfated dextran hydrogel at day 14, with sections at different planes revealing the formation of lumen structures, scale bar, 50 μm (left), and representative confocal fluorescent image of in vitro vascular network formation in highly sulfated dextran hydrogel at day 30, scale bar: 500 μm (right).

FIGS. 14A & 14B provide representative images and graphs showing quantitative analysis of host blood vessels invading into sulfated dextran gels (either low sulfation (LS) or high sulfation (HS)) based on percentage of mCD31 positive area (FIG. 14A), and percentage of perfused host vessels quantified by FITC-dextran (70 kDa) positive area, n>4 (FIG. 14B), scale bar, 200 μm.

### DETAILED DESCRIPTION

The present disclosure is based, in part, on the development of a synthetic polymer, e.g., synthetic heparin mimetic, comprising a polymeric carbohydrate backbone of repeating polysaccharide units, wherein the polysaccharide units comprise one or more chemically reactive hydroxyl groups, wherein one or more of the hydroxyl groups is modified by converting the hydroxyl groups with a negatively charged functional group to provide a negative charge to the synthetic polymer (e.g., by sulfation) to promote vascularization, growth factor binding and/or growth factor activity, e.g., similar to heparin, without the corresponding anti-coagulant activity, essentially de-coupling the two main functions of heparin. Without being bound by theory, it is believed that the length and flexible nature of the carbohydrate backbone, and the general hydrophilicity and negative charge of the synthetic polymer allows for interaction of the mimetic with cell-associated growth factors in vivo. By providing a negative charge to the synthetic polymer (e.g., by sulfation) it is believed that growth factor activity is enhanced relative to an unmodified polymer by for example, (a) inhibiting growth factor internalization by the cell; (b) maintaining the growth factor on the cell surface thereby enhancing cell signaling; (c) enhancing multimerization of growth factors by receptor clustering; and/or (d) acting as a sink to reduce growth factor diffusion from the cell surface.

The disclosure further provides a synthetic polymer, e.g., synthetic heparin mimetic, with enhanced angiogenesis and/or improved vascularization of a tissue or engineered tissue construct without the potential unwanted side effect of bleeding.

The disclosure is further based on the discovery that incorporating a synthetic polymer, e.g., synthetic heparin mimetic, into a hydrogel increases sprouting of vessels from cells within the hydrogel. Without being bound by theory, a hydrogel comprising the synthetic polymer, e.g., synthetic heparin mimetic, increases vascularization, survival and/or engraftment of cell implant or an engineered tissue construct in a subject. In some embodiments, a cell implant or an engineered tissue construct comprises the hydrogel described herein.

### I. Definitions

Terms used in the claims and specification are defined as set forth below unless otherwise specified.

As used herein and in the appended claims, the singular forms “a,” “and,” and “the” include plural referents unless the context clearly dictates otherwise. Thus, for example, reference to “the cell” includes reference to one or more cells known to those skilled in the art, and so forth.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

As used herein, the term “bioactive agent” can refer to any agent capable of promoting tissue formation, destruction, and/or targeting a specific disease state. Examples of bioactive agents can include, but are not limited to, chemotactic agents, various proteins (e.g., short term peptides, bone morphogenic proteins, collagen, glycoproteins, and lipoprotein), cell attachment mediators, biologically active ligands, integrin binding sequence, various growth and/or differentiation agents and fragments thereof (e.g., epidermal growth factor (EGF), hepatocyte growth factor (HGF), vascular endothelial growth factors (VEGF), fibroblast growth factors (e.g., bFGF), platelet derived growth factors (PDGF), insulin-like growth factor (e.g., IGF-I, IGF-II) and transforming growth factors (e.g., TGF-β I-III), parathyroid hormone, parathyroid hormone related peptide, bone morphogenic proteins (e.g., BMP-2, BMP-4, BMP-6, BMP-7, BMP-12, BMP-13, BMP-14), transcription factors, such as sonic hedgehog, growth differentiation factors (e.g., GDF5, GDF6, GDF8), recombinant human growth factors (e.g., MP52 and the MP-52 variant rhGDF-5), cartilage-derived morphogenic proteins (CDMP-1, CDMP-2, CDMP-3), small molecules that affect the upregulation of specific growth factors, tenascin-C, hyaluronic acid, chondroitin sulfate, fibronectin, decorin, thromboelastin, thrombin-derived peptides, heparin-binding domains, heparin, heparan sulfate, polynucleotides, DNA fragments, DNA plasmids, MMPs, TIMPs, interfering RNA molecules, such as siRNAs, oligonucleotides, proteoglycans, glycoproteins, glycosaminoglycans, and DNA encoding for shRNA.

As used herein, the term “cell implant” refers to a composition comprising a population of cells for implantation to a subject, aimed to restore or to replace the function of missing, damaged or diseased cells or tissue. The cell implant may be in the form of a liquid, e.g., a liquid solution of single cells, or may be in the form of a solid, e.g., a hydrogel. The cells comprised in a cell implant may be single cells, e.g., in liquid solution, or cell aggregates, e.g., in a hydrogel. In some embodiments, the cell implant may further comprise a growth factor. The cell implant may be a tissue e.g., a tissue graft harvested from a subject, or an engineered tissue construct.

As used herein, the term “coagulation,” also known as “blood coagulation,” blood clotting” and “clotting,” refers to the process by which blood changes from a liquid to a gel, forming blood clot. When a blood vessel is damaged and becomes leaky, coagulation is triggered to form a blood clot to seal the site of injury and prevent blood loss. The coagulation system functions under an intricate balance between coagulation factors in the blood ready to be activated when injury occurs and mechanisms to inhibit coagulation beyond the site of injury. Dysregulation of the coagulation system in a subject can either lead to excessive bleeding or clotting disorders such as thrombosis, stroke and pulmonary embolism.

Anti-coagulants are agents that impede blood coagulation usually by reducing the action of clotting factors directly or indirectly. Anti-coagulants are one type of agent often prescribed to subjects with excessive clotting in their circulation, such as people with high age. Commonly used anti-coagulations include vitamin K antagonists, thrombin inhibitors, factor Xa inhibitors and low molecular weight heparin.

Methods of measuring coagulation activity, e.g., anti-coagulant activity or pro-coagulant activity, of an agent are well-known in the art. Coagulation tests are also routinely used in clinical setting to assess to ability to clot by a subject's blood (see, e.g., Aria M M. et al. Front Bioeng Biotechnol. 2019; 7:395). In a non-limiting example, the anti-coagulant activity of an agent can be measured by mixing the agent with blood at varying concentration and determining the time it takes for blood to clot, also called blood clotting time. An agent has anti-coagulant activity, i.e., impairs blood coagulation, when blood clotting time in the presence of the agent increases compared to blood clotting time without the agent. An agent has pro-coagulant activity, i.e., enhances blood coagulation, when blood clotting time in the presence of the agent decreases compared to blood clotting time without the agent. An exemplary assay that may be used to measure coagulation activity of an agent includes a mouse tail bleeding assay as described herein in Example 5.

As used herein, the term “co-culture” refers to a collection of cells cultured in a manner such that more than one population of cells are in association with each other. Co-cultures can be made such that cells exhibit heterotypic interactions (i.e., interaction between cells of populations of different cell types), homotypic interactions (i.e., interaction between cells of the same cell types) or co-cultured to exhibit a specific and/or controlled combination of heterotypic and homotypic interactions between cells.

As used herein, the terms “cross-linked” and “linked” are used interchangeably and refer to an attachment of two chains of polymer molecules by bridges, composed of either an element, a group, or a compound, that join certain atoms of the chains by chemical bonds. Cross-linking can be effected naturally and artificially. Internal cross-linking between two sites on a single polymer molecular is also possible.

The terms “cross-linker” or “cross-linking agent”, as used herein, refers to the element, group, or compound that effects cross-linking between polymer chains.

As used herein, the term “degree of sulfation” refers to the number of sulfate groups per monosaccharide unit of a polysaccharide.

As used herein, the term “ectopic” means occurring in an abnormal position or place. Accordingly, “implantation at an ectopic site” means implantation at an abnormal site or at a site displaced from the normal site. Exemplary ectopic sites of implantation include, but are not limited to the intraperitoneal space and ventral subcutaneous space. Ectopic sites of implantation can also be within an organ, i.e., an organ different than that of the source cells of the construct being implanted (e.g., implanting a human liver construct into the spleen of an animal). Ectopic sites of implantation can also include other body cavities capable of housing a construct described herein. In some embodiments, ectopic sites include, for example, lymph nodes. The term “ectopic” and “heterotropic” can be used interchangeably herein.

As used herein, the term “encapsulation” refers to the confinement of a cell or population of cells within a material, in particular, within a biocompatible hydrogel. The term “co-encapsulation” refers to encapsulation of more than one cell or cell type or population or populations of cells within the material, e.g., the hydrogel.

As used herein, the term “functional group” refers to an atom or group of atoms within a molecule that has similar chemical properties whenever it appears in various compounds. The same functional group will undergo the same or similar chemical reaction(s) regardless of the size of the molecule it is part of.

As used herein, the term “growth factor” refers to a molecule that elicits a biological response to improve tissue regeneration, tissue growth and organ function.

The terms “heparin” and “heparan sulfate” refer generally to any preparation isolated from a mammalian tissue in a manner conventional for the preparation of heparin as an anticoagulant, or to any preparation otherwise obtained or synthesized and corresponding to that obtained from tissue. Such preparations are composed of repeating units of D-glucosamine and either L-iduronic or D-glucuronic acids. The size and precise nature of the polymeric chains and the degree of sulfation in heparin varies from preparation to preparation, and the terms “heparin” and “heparin sulfate” are intended to cover all such preparations.

As used herein, the term “heparin mimetic” refers to a molecule having at least one function of heparin. In some embodiments, the heparin mimetic shares structural features of heparin. In some embodiments, the heparin mimetic has the same or substantially the same negative charge as heparin. In some embodiments, the synthetic heparin mimetic is capable of binding growth factors to the same or substantially the same extent as heparin. In some embodiments, the synthetic heparin mimetic has reduced anti-coagulation activity relative to heparin.

As used herein, the term “hydrogel” refers to a network of polymer chains that are hydrophilic in nature, such that the material absorbs a high volume of water or other aqueous solution. Hydrogels can include, for example, at least 70% v/v water, at least 80% v/v water, at least 90% v/v water, at least 95%, 96%, 97%, 98% and even 99% or greater v/v water (or other aqueous solution). Hydrogels can comprise natural or synthetic polymers, the polymeric network often featuring a high degree of crosslinking. Hydrogels also possess a degree of flexibility very similar to natural tissue, due to their significant water content. Hydrogels are particularly useful in tissue engineering applications as scaffolds for culturing cells. In certain embodiments, the hydrogels are made of biocompatible polymers.

As used herein, the term “homopolymer” refers to a molecule having the same repeating monosaccharide unit.

As used herein, the term “modified dextran” refers to a dextran molecule comprising one or more chemically reactive hydroxyl groups modified with a functional group (e.g., sulfate).

“Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “polysaccharide” refers to a polymer comprising repeating monosaccharide, disaccharide or polysaccharide units bound together by glycosidic linkages. This term encompasses any polysaccharide, including synthetically derived polysaccharides and naturally occurring polysaccharides that are found in nature. Naturally occurring polysaccharides may be isolated and purified from natural sources, e.g., plants, algae, animal bacteria, and fungi. Non-limiting examples of polysaccharides include starch, cellulose, glucomannan, pectin, hemicellulose, gums, mucilage, agar, galactans, alginates, carrageenans, chitin, chitosan, hyaluronic acid, glycosaminoglycans, galactogen, dextran, inulin, levan, polygalactosamine, gellan, xanthan, elsinan, pectin, pullulan and yeast glucans. In some embodiments, the polysaccharide is selected from the group consisting of dextran, hyaluronic acid, galactogen, inulin and pectin. In one specific embodiment, the polysaccharide is dextran. In some embodiments, the polysaccharide serves as a starting material for generating a synthetic polymer of the disclosure by modifying the polysaccharide to convert one or more functional groups present in the polysaccharide into negatively charged functional groups. Naturally occurring polysaccharides may also be artificially synthesized.

As used herein, the term “monosaccharide”, “monosaccharide unit”, which may be used interchangeably with the term “monosaccharide monomer”, refers to the simplest carbohydrate that cannot be hydrolyzed into smaller carbohydrates.

As used herein, the term “disaccharide” refers to a carbohydrate consisting of two monosaccharide monomers.

As used herein, the term “negatively charged functional group” refers to a functional group that comprises a negative charge. Exemplary negatively charged functional groups include a sulfate group, a phosphate group and a carboxylic group.

As used herein, the term “polysaccharide unit” refers to a molecule comprising more than two monosaccharide monomers. A polysaccharide unit will vary in size depending on the characteristic and number of monomers.

As used herein, the term “sulfation” refers to a transfer of a sulfonate or sulfuryl group from one molecule to another.

As used herein, the term “sulfation site” refers to functional groups that can be sulfated. Preferably, the functional group is a hydroxyl group or an amino group. As used herein, “sulfation site” includes both free functional groups that can be sulfated and functional groups that already have sulfate groups.

As used herein, the term “vascularization” refers to the formation of blood vessels. In order for a tissue, e.g., an engineered tissue for implantation, to survive, a connecting network of blood vessels, i.e., a vascular network (e.g., a capillary network), has to form to provide sufficient blood supply and necessary nutrients to cells in the tissue. Insufficient vascularization can lead to improper cell integration or cell death in an implanted tissue. Blood vessel formation is classically divided into 2 categories: vasculogenesis and angiogenesis. As used herein, the term “vasculogenesis” refers to the de novo formation of blood vessels from precursor cells (e.g., endothelial cells). The term “angiogenesis” refers to the formation of blood vessels from preexisting vessels.

Assays that can be used to assess and quantify vascularization are well known in the art. For example, vascularization promoting activity of an agent, e.g., a synthetic polymer of the disclosure, can be assessed by assessing endothelial cell survival, proliferation, migration and morphogenesis after the cell has been contacted with the agent. An exemplary assay that may be used to assess vasculogenesis involves culturing human umbilical vein endothelial cells (HUVEC) in a matrix scaffold in the presence of growth factors. Under appropriate conditions, endothelial cells migrate and form a network of chords or tubes. Quantification of properties such as the length or area covered by chords/tubes per unit area, or number of branching per area can be used as measurements of vasculogenesis. Another exemplary assay that may be used to assess angiogenesis common assay is a sprouting assay, wherein endothelial cells are cultured as spheroids or aggregates in a matrix and angiogenesis is determined by the number and length of sprouts formed from the cell spheroids. Discussion of additional assays that may be used to assay vascularization may be found in Goodwin, Microvasc Res. 74(2-3): 172-183 (2007) and Tahergorabi and Khazaei et al. Iran J Basic Med Sci. 15(6): 1110-1126 (2012).

As used herein, the term “promote vascularization” refers to the ability of an agent to support, e.g., increase and/or accelerate, blood vessel formation. A number of growth factors or cytokines (e.g., VEGF, PDGF, FGF, TGF-β and angiopoietin) and extracellular matrix proteins (e.g., collagen I, fibrin) are known to promote vascularization.

As used herein, the term “synthetic polymer promotes vascularization”, refers to ability of the synthetic polymer of the disclosure to support, e.g., induce, increase and/or accelerate, blood vessel formation. In some embodiments, the term “synthetic polymer promotes vascularization” refers to the ability of the synthetic polymer of the disclosure to support an amount of vascularization that is substantially similar to an amount of vascularization supported by a comparable amount of heparin. For example, the synthetic polymer of the disclosure may support an amount of vascularization that is at least 10%, at least 25%, at least 50%, at least 75% or at least 90% of the amount of vascularization supported by a comparable amount of heparin.

As used herein, the language “synthetic polymer promotes greater vascularization than a corresponding polysaccharide that has not been modified by converting hydroxyl groups into negatively charged functional groups” means that the synthetic polymer of the disclosure supports an amount of vascularization that is at least 10%, at least 20%, at least 30%, at least 50%, at least 75% or at least 100% greater than vascularization supported by a corresponding polysaccharide that has not been modified by converting hydroxyl groups into negatively charged functional groups.

As used herein, the term “promote binding of growth factors”, when used in reference to the synthetic polymer of the disclosure, refers to the ability of the synthetic polymer of the invention to bind to one or more growth factors. Without wishing to be bound by a specific theory, it is believed that the binding affinity of a synthetic polymer of the disclosure may be correlated with the amount of negative charge present in the synthetic polymer. For example, a synthetic polymer of the disclosure comprising a higher amount of negatively charged functional groups, e.g., an average of 2 negatively charged functional groups per monosaccharide unit, may be characterized by a higher binding affinity to one or more growth factors than a synthetic polymer comprising a lower amount, of negatively charged functional groups, e.g., an average of 0.5 negatively charged functional groups per monosaccharide unit.

In some embodiments, a synthetic polymer of the disclosure is characterized by a binding affinity to one or more growth factors that is comparable to the binding affinity of heparin to the one or more growth factors. For example, a synthetic polymer of the disclosure may be characterized by a binding affinity to one or more growth factors that is at least 10%, at least 20%, at least 30%, at least 50%, at least 75% or at least 90% of the binding affinity of the one or more growth factors to heparin.

As used herein, the language “synthetic polymer promotes greater binding of one or more growth factors as compared to a corresponding polysaccharide that has not been modified by converting the hydroxyl groups into negatively charged functional groups” means that binding affinity of the synthetic polymer of the disclosure to one or more growth factors is least 10%, at least 20%, at least 30%, at least 50%, at least 75%, or at least 100% higher than binding affinity to one or more growth factors of a corresponding polysaccharide that has not been modified by converting hydroxyl groups into negatively charged functional groups.

As used herein, the term “growth factor activity” encompasses growth factor dependent cell signaling activity. A growth factor dependent cell signaling activity may be measured, e.g., by measuring an amount of phosphorylation of one or more proteins involved in a cell signaling pathway modulated by the growth factor. For example, activity of VEGF may be measured by determining an amount of phosphorylation of VEGF receptor, and/or phosphorylation of one or more proteins that function downstream of VEGF receptor signaling, such as ERK1/2 and Akt, as described in Example 6.

As used herein, the language “promote growth factor activity”, when used in reference to a synthetic polymer of the disclosure, refers to the ability of the synthetic polymer of the disclosure to support, e.g., induce and/or increase activity of growth factors in the presence of the synthetic polymer of the disclosure. In some embodiments, the activity of one or more growth factors in the presence of a synthetic polymer of the disclosure is comparable to the activity of the one or more growth factors in the presence of heparin. For example, activity of one or more growth factors in the presence of a synthetic polymer of the disclosure, e.g., as measured by phosphorylation of one or more proteins involved in a cell signaling pathway modulated by the growth factor, may be at least 10%, at least 25%, at least 50%, at least 75% or at least 90% of the activity of the one or more growth factors in the presence of a comparable amount of heparin.

As used herein, the language “synthetic polymer promotes growth factor activity to a greater extent than a corresponding polysaccharide that has not been modified by converting hydroxyl groups into negatively charged functional groups” means that activity of one or more growth factors in the presence of a synthetic polymer of the disclosure is at least 10%, at least 20%, at least 30%, at least 50%, at least 75%, or at least 100% higher than activity of the one or more growth factors in the presence of a corresponding polysaccharide that has not been modified by converting hydroxyl groups into negatively charged functional groups.

As used herein, the language “does not impair blood coagulation” refers to an agent, e.g., a synthetic polymer of the disclosure, that does not inhibit blood coagulation. In some embodiments, the agent, e.g., a synthetic polymer of the disclosure, causes a higher amount of blood coagulation than amount of blood coagulation observed in the presence of a comparable amount of heparin. That is, an agent, e.g., a synthetic polymer of the disclosure, that does not impair blood coagulation is an agent that may reduce an amount of blood coagulation to a lesser extent than a comparable amount of heparin. For example, an agent that does not impair blood coagulation, e.g., a synthetic polymer of the disclosure, may cause an amount of blood coagulation that is at least about 10% higher, e.g., at least about 25%, at least about 50% higher, at least about 75% higher, or at least about 100% higher than the amount of blood coagulation observed in the presence of a comparable amount of heparin.

An amount of blood coagulation may be measured by any method known in the art for measuring blood coagulation, for example, by a tail bleeding assay. Thus, a tail bleeding time in the presence of a synthetic polymer of the disclosure may be at least about 10% shorter, at least about 25% shorter, at least about 50% shorter, or at least about 75% shorter than the tail bleeding time in the presence of a comparable amount of heparin.

In some embodiments, a synthetic polymer of the present disclosure that does not impair blood coagulation comprises an average of less than 2 negatively charged functional groups per monosaccharide.

As used herein, the term “growth factor dependent cell therapy” refers to cell therapy that comprises therapeutic use of cells responsive to one or more growth factors. The term ‘treatment of a patient in need of an implant’ as used herein refers to a treatment aiming to restore or to replace the function of a missing tissue and wherein the provision of the hydrogel described herein is aimed at improving regeneration of a damaged tissue wherein said implant is implanted. In other embodiments, the treatment is aimed at the sustained or extended release of a medicament or drug incorporated in said hydrogel.

### II. Synthetic Polymers

The present disclosure provides a synthetic polymer comprising a polysaccharide that has been modified by converting one or more groups present in the polysaccharide into negatively charged functional groups, wherein the negatively charged groups provide an amount of negative charge to the synthetic polymer sufficient to promote one or more of binding of growth factors, growth factor activity and vascularization. In some embodiments, the functional groups in the polysaccharide that are converted into the negatively charged groups are hydroxyl groups.

In some embodiments, the present disclosure provides a synthetic polymer comprising a polysaccharide comprising hydroxyl groups, wherein one or more of the hydroxyl groups has been modified by converting the hydroxyl groups into negatively charged functional groups, wherein the negatively charged groups provide an amount of negative charge to the synthetic polymer sufficient to promote one or more of binding of growth factors, growth factor activity and vascularization.

In some embodiments, the disclosure provides a synthetic polymer comprising a polymeric carbohydrate backbone of repeating polysaccharide units, each unit having one or more hydroxyl groups, wherein the synthetic polymer is modified at one or more hydroxyl groups (e.g., one or more hydroxyl groups in a each repeating polysaccharide unit) with a functional group to provide a negative charge to the synthetic polymer.

In some embodiments, the synthetic polymer is characterized by a zeta potential of −20 mV or less, e.g., about −20 mV to about −60 mV. In some embodiments, the synthetic polymer of the present disclosure comprises an average of 0.5 to 2 negatively charged groups per monosaccharide unit.

In some embodiments, the synthetic polymer is a synthetic heparin mimetic. Heparin sulfates (HSs) are highly sulfated polysaccharides present on the surface of mammalian cells and in the extracellular matrix in large quantities. HS is a highly charged polysaccharide comprising 1 to 4-linked glucosamine and glucuronic/iduronic acid units that contain both N- and O-sulfo groups. Heparin, a specialized form of HS, is a commonly used anticoagulant drug. Thus, “heparan sulfate”, as used herein, includes heparin.

Heparin is a polysaccharide that comprises a disaccharide-repeating unit of either iduronic acid (IdoA) or glucuronic acid (GlcA) and glucosamine residues, each capable of carrying sulfo groups. The locations of the sulfo groups, IdoA and GlcA dictate the anticoagulant activity of heparin. In vivo, heparin is synthesized by a series of heparan sulfate (HS) biosynthetic enzymes. HS polymerase catalyzes the formation of the polysaccharide backbone, a repeating disaccharide of GlcA and N-acetylated glucosamine (GlcNAc). This backbone is then modified by N-deacetylase/N-sulfotransferase (NDST), C5-epimerase (C5-epi), 2-O-sulfotransferase (2-OST), 6-O-sulfotransferase (6-OST), and 3-O-sulfotransferase (3-OST).

Heparins play roles in a variety of important biological processes, including assisting viral infection, regulating blood coagulation and embryonic development, and suppressing tumor growth. The biosynthesis of heparin occurs in the Golgi apparatus. It can initially be synthesized as a copolymer of glucuronic acid and N-acetylated glucosamine by D-glucuronyl and N-acetyl-D-glucosaminyltransferase, followed by various modifications (Lindahl, U., et al., (1998) J. Biol. Chem. 273:24979-24982). These modifications can include N-deacetylation and N-sulfation of glucosamine, C5 epimerization of glucuronic acid to form iduronic acid residues, 2-O-sulfation of iduronic and glucuronic acid, as well as 6-O-sulfation and 3-O-sulfation of glucosamine. Several enzymes that are responsible for the biosynthesis of heparan sulfate have been cloned and characterized (Esko, J. D., and Lindahl, U. (2001) J. Clin. Invest. 108:169-173).

In some embodiments, the disclosure provides a synthetic polymer, e.g., synthetic heparin mimetic, comprising a polymeric carbohydrate backbone of repeating polysaccharide units, each unit having one or more chemically reactive hydroxyl groups, wherein the synthetic polymer, e.g., synthetic heparin mimetic, is modified at the one or more hydroxyl groups with a functional group to provide a negative charge to the synthetic polymer, e.g., synthetic heparin mimetic. The term “chemically reactive hydroxyl groups” refers to hydroxyl groups present in the polymeric carbohydrate backbone that are capable of being modified with a functional group to provide a negative charge to the synthetic polymer. The term “hydroxyl groups” is used interchangeably with the term “chemically reactive hydroxyl groups” herein.

In some embodiments, a synthetic polymer of the disclosure, e.g., synthetic heparin mimetic, is generated by providing a saccharide substrate, elongating the saccharide substrate to a polysaccharide of a desired or predetermined length, and performing one or more chemical reactions with a functional group to negatively charge the polysaccharide. In some embodiments, a synthetic polymer of the disclosure, e.g., synthetic heparin mimetic, is generated by providing a polysaccharide, e.g., that has been isolated and purified from a cell or biological material, and performing one or more chemical reactions with a functional group to negatively charge the polysaccharide.

In some embodiments, the synthetic polymer of the disclosure, e.g., synthetic heparin mimetic, has a negative (surface) charge. Native heparin binds to various growth factors, extracellular matrix proteins, chemokines, etc. through the electrostatic interaction of highly negative charges. In some embodiments, the synthetic polymer of the disclosure, e.g., synthetic heparin mimetic, has the same or substantially the same negative charge as heparin. In some embodiments, the negative charge is measured by determining the zeta potential. An exemplary method for measuring zeta potential comprises applying a controlled electric field to a sample via electrodes immersed in the same. The electric field causes charged particles to move towards the electrode of opposite polarity. In some embodiments, zeta potential is calculated with Smoluchowski's formula. In some embodiments, units for zeta potential is millivolts (mV). In some embodiments, the zeta potential of a synthetic polymer, e.g., synthetic heparin mimetic, is the same or substantially the same zeta potential as heparin. In some embodiments, the zeta potential of a synthetic polymer, e.g., synthetic heparin mimetic is between −10 mV and −20 mV, −10 mV and −30 mV, −10 mV and 50 mV, −10 mV and −60 mV, −20 mV and −30 mV, −20 mV and −50 mV, −20 mV and −70 mV, −30 mV and −40 mV, −30 mV and −50 mV, −30 mV and −60 mV, −40 mV and −50 mV, −50 mV and −60 mV, and −60 mV and −70 mV. In some embodiments, the negative charge of the synthetic polymer, e.g., synthetic heparin mimetic, is sufficient for interacting with (e.g., binding) growth factors. In some embodiments, a higher negative charge of the synthetic polymer, e.g., synthetic heparin mimetic, (e.g., −40 to −50 mV, −20 to −30 mV vs. −10 to −20 mV) allows for enhanced interaction (e.g., binding) with growth factors.

In some embodiments, a synthetic polymer of the disclosure, e.g., synthetic heparin mimetic, binds a growth factor to the same or similar extent as heparin. In some embodiments, a synthetic polymer of the disclosure, e.g., synthetic heparin mimetic, has reduced anti-coagulant activity relative to heparin. In some embodiments, the synthetic polymer, e.g., synthetic heparin mimetic, has reduced anti-coagulant activity relative to heparin. Methods of measuring anti-coagulant activity or coagulant activity of an agent are well-known in the art. Coagulation tests are also routinely used in a clinical setting to assess to ability to clot by a subject's blood (see, e.g., Aria M M. et al. Front Bioeng Biotechnol. 2019; 7:395). In a non-limiting embodiment, the anti-coagulant activity of an agent, e.g., heparin, or synthetic polymer, e.g., synthetic heparin mimetic, may be measured by mixing the agent with blood at varying concentration and determining blood clotting time. In some embodiments, the synthetic polymer, e.g., synthetic heparin mimetic, has a similar anti-coagulant activity to heparin. In some embodiments, the negative charge of the synthetic polymer, e.g., synthetic heparin mimetic, is sufficient for anti-coagulant activity. In some embodiments, the synthetic polymer having similar anti-coagulant activity to heparin has a the zeta potential of about −50 to about −60 mV.

In some embodiments, the polysaccharide of the synthetic polymer, e.g., synthetic heparin mimetic, comprises the same repeating unit. In some embodiments, the polysaccharide comprising the same repeating unit is a homopolymer. In some embodiments, the polysaccharide of the synthetic polymer, e.g., synthetic heparin mimetic, comprises two different repeating units. In some embodiments, the polysaccharide of the synthetic polymer, e.g., synthetic heparin mimetic, comprises three or more different repeating units. In some embodiments, the polysaccharide comprises repeating monoccharide units, e.g., prior to modification of the hydroxyl groups. In some embodiments, the polysaccharide comprises repeating disaccharide units, e.g., prior to modification of the hydroxyl groups. In some embodiments, the polysaccharide comprises repeating polyccharide units, e.g., prior to modification of the hydroxyl groups.

In some embodiments, the polysaccharide is linear.

In some embodiments, the polysaccharide of the synthetic polymer, e.g., synthetic heparin mimetic, is dextran, alginate, agarose, chondroitin sulfate, chitin/chitosan, cellulose, starch, hyaluronic acid, galactogen, inulin, pectin or glycogen. In some embodiments, the polysaccharide is dextran. In some embodiments, the polysaccharide is dextran. In some embodiments, the polysaccharide is alginate. In some embodiments, the polysaccharide is agarose. In some embodiments, the polysaccharide is chondroitin sulfate. In some embodiments, the polysaccharide is chitin/chitosan. In some embodiments, the polysaccharide is cellulose. In some embodiments, the polysaccharide is starch. In some embodiments, the polysaccharide is hyaluronic acid. In some embodiments, the polysaccharide is galactogen. In some embodiments, the polysaccharide is inulin. In some embodiments, the polysaccharide is pectin. In some embodiments, the polysaccharide is glycogen.

In some embodiments, one or more hydroxyl groups present in the polysaccharide are modified by converting them into negatively charged functional groups. In some embodiments, at least 5%, at least about 10%, at least about 20%, at least 30%, at least about 40%, at least about 50%, at least about 60%, at least about 70%, at least about 80%, or at least about 90% of hydroxyl groups present in the polysaccharide are modified by converting them into negatively charged functional groups.

In some embodiments, the synthetic polymer, e.g., synthetic heparin mimetic, comprises an average of at least 0.1, e.g., at least 0.2, at least 0.5, at least 0.8, at least 1, at least 1.5 or at least 2 negatively charged functional groups per monosaccharide unit. In some embodiments, the synthetic polymer comprises an average of 0.1 to 2.0 negatively charged functional groups per monosaccharide unit. In some embodiments, the synthetic polymer comprises an average of 0.1 to 0.25, 0.25 to 0.5, 0.3 to 0.6, 0.5 to 1.0, 0.5 to 1.5, 0.5 to 3.0, 1.0 to 1.5, 1.5 to 2.0, 1.0 to 3.0, or 2.0 to 3.0 negatively charged functional groups per monosaccharide unit.

In some embodiments, the synthetic polymer is characterized by greater binding of growth factors than the unmodified polysaccharide. In some embodiments, the synthetic polymer can bind a greater number of growth factor molecules, or can bind growth factor molecules with higher affinity, as compared to the unmodified polysaccharide.

In some embodiments the synthetic polymer binds at least 10%, 20%, 30%, 40%, 50%, 60%, 70%, or 80% more growth factor molecules than the unmodified polysaccharide. In some embodiments the synthetic polymer binds a growth factor molecule with at least 10%, 20% 30%, 40%, 50%, 60%, 70% or 80% higher affinity than the unmodified polysaccharide.

Dextran

In some embodiments, the synthetic polymer, e.g., synthetic heparin mimetic, comprises dextran as the polysaccharide. Dextrans (α-1,6-glucan) are viscous glucans, which mainly comprise α-1,6 linkages, and may be produced from sucrose by Leuconostoc mesenteroides and such, which belong to lactic acid bacteria. Typically, dextran is synthesized by transferring a glucose residue from a sucrose molecule to the primer via an α-1,6-linkage, by the action of dextran sucrase. To date, several dozen types of dextran-producing bacteria have been found. Though the α-1,6 linkage content varies according to the bacterial strain, glucans comprising 65% or more α-1,6 linkage content are generally called “dextrans”. α-1,3 and α-1,2 linkages are also comprised as other linkages, but most are present as branches. The monomer of dextran is C₆H₁₀O₅.

Dextran is represented by the following structure in the following schematic having 3 reactive hydroxyl groups (“C2, C3 and C4”) on each monosaccharide unit, wherein n is 100-1000, 200-800, 300-600 or 400-500 (repeating units), Due to the structure and space availability, the reactive preference of —OH (hydroxyl) is C2>C4>C3:

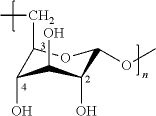

The monosaccharide unit of dextran has 3 reactive hydroxyl groups on 3 backbone carbons (“C2, C3 and C4”), as shown in schematic A. In some embodiments, the synthetic polymer comprises a plurality of monosaccharide units, wherein each monosaccharide unit independently may have one, two, all three, or none of the reactive hydroxyl groups on C2, C3 and C4 modified and replaced with a functional group. In some embodiments, the functional group is independently selected from a sulfate group, a phosphate group, a carboxylic group, other negatively charged moieties, and mixtures thereof.

Without wishing to be bound by theory, in some embodiments, in one or more monosaccharide units of dextran (at least 10%, 20%, 30%, 40%, 50%, 60%, 70%, 80% or more of the monosaccharide units), the hydroxyl group on C2 is replaced by a sulfate group, a phosphate group or a carboxylic group, and the hydroxyl groups on C3 and C4 are unmodified. In some embodiments, in one or more monosaccharide units of dextran (at least 10%, 20%, 30%, 40%, 50%, 60%, 70%, 80% or more of the monosaccharide units), the hydroxyl group on C4 is replaced by a sulfate group, a phosphate group or a carboxylic group, and the hydroxyl groups on C2 and C3 are unmodified. In some embodiments, in one or more monosaccharide units of dextran (at least 10%, 20%, 30%, 40%, 50%, 60%, 70%, 80% or more of the monosaccharide units), the hydroxyl groups on C2 and C4 are replaced by sulfate groups, phosphate group or carboxylic groups, and the hydroxyl groups on C3 is unmodified. In some embodiments, in one or more monosaccharide units of dextran (at least 10%, 20%, 30%, 40%, 50%, 60%, 70%, 80% or more of the monosaccharide units), the hydroxyl group on each of C2, C3 and C4 are replaced by sulfate groups, phosphate group or carboxylic groups.

In some embodiments, in one or more monosaccharide units of dextran (at least 10%, 20%, 30%, 40%, 50%, 60%, 70%, 80% or more of the monosaccharide units), the hydroxyl group on C2 is replaced by a sulfate group, a phosphate group or a carboxylic group, and the hydroxyl groups on C3 and/or C4 are replaced by a sulfate group, a phosphate group or a carboxylic group, wherein the functional groups on C3 and/or C4 are different from the function group on C2. In some embodiments, in one or more monosaccharide units of dextran (at least 10%, 20%, 30%, 40%, 50%, 60%, 70%, 80% or more of the monosaccharide units), the hydroxyl group on C4 is replaced by a sulfate group, a phosphate group or a carboxylic group, and the hydroxyl groups on C2 and/or C3 are replaced by a sulfate group, a phosphate group or a carboxylic group, wherein the functional groups on C2 and/or C3 are different from the function group on C4. In some embodiments, in one or more monosaccharide units of dextran (at least 10%, 20%, 30%, 40%, 50%, 60%, 70%, 80% or more of the monosaccharide units), the hydroxyl groups on C2, C3 and C4 are not converted to a functional group.

In certain aspects, the synthetic polymer comprises monosaccharide units wherein the functional groups on C2, C3 and C4 are not the same across all units.

In some embodiments, dextran has a mean weight-average molecular weight of 10-150 kDa. In some embodiments, dextran has a mean weight-average molecular weight of 20-50 kDa, 30-50 kDa, 50-100 kDa, 60-90 kDa, 70-90 kDa, or 80-100 kDa. In some embodiments, dextran has a mean weight-average molecular weight of 150-650 kDa, 150-400 kDa, or 400-650 kDa. In some embodiments, dextran comprises 50 to 500 monomers. In some embodiments, dextran comprises 400 to 500 or 400 to 600 monomers.

In some embodiments, a chemically reactive hydroxyl group of the polysaccharide, e.g., dextran, is modified with a functional group. In some embodiments, polysaccharide, e.g., dextran comprises a hydroxyl group at carbons C2, C3 and C4. In some embodiments, the hydroxyl group at carbon C2 is modified with a functional group. In some embodiments, the hydroxyl group at carbon C3 is modified with a functional group. In some embodiments, the hydroxyl group at carbon C4 is modified with a functional group. In some embodiments, the hydroxyl groups at carbons C2 and C3 is modified with a functional group. In some embodiments, the hydroxyl groups at carbons C2 and C4 is modified with a functional group. In some embodiments, the hydroxyl groups at carbons C3 and C4 is modified with a functional group. In some embodiments, the hydroxyl groups at carbons C2, C3 and C4 is modified with a functional group.

In some embodiments, at least 15% to 90% of chemically reactive hydroxyl groups are modified with a functional group. In some embodiments, at least 50% of chemically reactive hydroxyl groups are modified with a functional group. In some embodiments, at least 70% of chemically reactive hydroxyl groups are modified with a functional group. In some embodiments, dextran comprises 400 to 500 monomers with 15% to 90% of chemically reactive hydroxyl groups modified with a functional group (e.g., sulfate).

In some embodiments, the synthetic polymer, e.g., synthetic heparin mimetic, comprises a polysaccharide selected from any of the polysaccharides disclosed herein. It will be understood that the polysaccharide may, depending on the particular monosaccharide units comprised in the polysaccharide, contain monosaccharide units having 1, 2, or 3 reactive hydroxyl groups (carbon positions “C2, C3 and C4”) on the monosaccharide unit. It will be understood that any hydroxyl group present on the monosaccharide units of the polysaccharide may be modified by converting the hydroxyl group to a negatively charged functional group disclosed herein, similarly as described for dextran.

In some embodiments, a chemically reactive hydroxyl group of the polysaccharide is modified with a functional group. In some embodiments, the polysaccharide comprises a hydroxyl group at carbons C2, C3 and C4. In some embodiments, the hydroxyl group at carbon C2 is modified with a functional group. In some embodiments, the hydroxyl group at carbon C3 is modified with a functional group. In some embodiments, the hydroxyl group at carbon C4 is modified with a functional group. In some embodiments, the hydroxyl groups at carbons C2 and C3 is modified with a functional group. In some embodiments, the hydroxyl groups at carbons C2 and C4 is modified with a functional group. In some embodiments, the hydroxyl groups at carbons C3 and C4 is modified with a functional group. In some embodiments, the hydroxyl groups at carbons C2, C3 and C4 is modified with a functional group.

In some embodiments, at least 15% to 90% of chemically reactive hydroxyl groups are modified with a functional group. In some embodiments, at least 50% of chemically reactive hydroxyl groups are modified with a functional group. In some embodiments, at least 70% of chemically reactive hydroxyl groups are modified with a functional group. In some embodiments, dextran comprises 400 to 500 monomers with 15% to 90% of chemically reactive hydroxyl groups modified with a functional group (e.g., sulfate).

Functional Groups

In some embodiments, the functional group providing a negative charge to the synthetic polymer, e.g., synthetic heparin mimetic, is selected from a sulfate group, a phosphate group, a carboxylic group, and any combination thereof. In some embodiments, a chemically reactive hydroxyl group of dextran is modified with the functional group.

In some embodiments, the synthetic polymer, e.g., synthetic heparin mimetic, comprises a sulfate group. Methods for adding a sulfate group to a polysaccharide are known to those of skill in the art, and are described, for example, in WO 2003020735, incorporated herein by this reference. Sulfotransferases comprise a family of enzymes that catalyze the transfer of a sulfonate or sulfuryl group (SO3) from a sulfo donor compound, i.e. an SO3-donor molecule, to an acceptor molecule. Sulfotransferases mediate sulfation of different classes of substrates such as carbohydrates, oligosaccharides, peptides, proteins, flavonoids, and steroids for a variety of biological functions including signaling and modulation of receptor binding. In some embodiments, a sulfate is added by dissolving the starting saccharide salt is in a dipolar aprotic solvent, optionally selected from the group consisting of pyridine, pyridine-dimethyl formamide (DMF), and pyridine-dimethylsulfoxide (DMSO), and is treated with a sulfating agent.

In some embodiments, the synthetic polymer, e.g., synthetic heparin mimetic, comprises a phosphate group. Methods for adding a phosphate group to a polysaccharide are known to those of skill in the art, and are described, for example, in US 20060154896, incorporated herein by this reference.

In some embodiments, the synthetic polymer, e.g., synthetic heparin mimetic, comprises a carboxylic group. Methods for adding a carboxylic group to a polysaccharide are known to those of skill in the art, and are described, for example, in WO 200002788, incorporated herein by this reference.

### Synthetic Polymer-Based Hydrogel

In some embodiments, the disclosure provides a hydrogel comprising a plurality of synthetic polymers, e.g., synthetic heparin mimetics, described herein. In some embodiments, the plurality of synthetic polymers, e.g., synthetic heparin mimetics, are cross-linked as described herein to form the hydrogel. In some embodiments, one or more bioactive agents are further added to modify the function of the hydrogel for various biomedical applications.

In some embodiments, the hydrogel described herein can promote vascularization, e.g., angiogenesis. In some embodiments, the hydrogel comprising the synthetic polymers of the disclosure promotes vascularization, e.g., angiogenesis, better than the hydrogel comprising unmodified polysaccharide.

In vitro assays to assess the effect of an agent on vascularization, e.g., angiogenesis, are well known in the art. Angiogenic activity of an agent can be assessed by observing the effect of the agent on endothelial cell survival, proliferation, migration and morphogenesis. An often used assay involves culturing human umbilical vein endothelial cells (HUVEC) in a matrix scaffold in the presence of growth factor. Under appropriate conditions, endothelial cells migrate and form a network of chords or tubes. Quantification of properties such as the length or area covered by chords/tubes per unit area, or number of branching per area can be used as measurements of angiogenesis. Another common assay is sprouting angiogenesis, wherein endothelial cells are cultured as spheroids or aggregates in a matrix and angiogenesis is often determined by the number and length of sprouts formed from the cell spheroids. Discussion of additional assays which may be used to assess vascularization may be found in Goodwin, Microvasc Res. 74(2-3): 172-183 (2007) and Tahergorabi and Khazaei et al. Iran J Basic Med Sci. 15(6): 1110-1126 (2012), the entire contents of which are incorporated herein.

In some embodiments, the present disclosure provides a hydrogel comprising a plurality of the synthetic polymers provided herein, wherein the synthetic polymers are cross-linked to each other by a cross-linker.

In some embodiments, the present disclosure provides a hydrogel comprising a plurality of synthetic polymers cross-linked to each other by a cross-linker, wherein each of said synthetic polymers comprises a polysaccharide comprising hydroxyl groups; wherein one or more of said hydroxyl groups has been modified by converting the hydroxyl groups into negatively charged functional groups. In some embodiments, the present disclosure provides hydrogel comprising a plurality of synthetic dextran polymers cross-linked to each other by a cross-linker, wherein each of said synthetic dextran polymers comprises a dextran polymer in which one or more hydroxyl groups naturally present in dextran has been modified by converting the hydroxyl groups into negatively charged functional groups. In some embodiments, the negatively charged functional groups provide an amount of negative charge to the synthetic polymer that is sufficient to promote one or more binding of growth factors, growth factor activity, and vascularization, or wherein the synthetic polymer has a zeta potential of about −10 mV to about −60 mV.

In some embodiments, said cross-linker is a non-covalent cross-linker. In some embodiments, said cross-linker is an ionic cross-linker. In some embodiments, said cross-linker is a covalent cross-linker. In some embodiments, said cross-linker is a peptide cross-linker. In some embodiments, said cross-linker is a cleavable cross-linker. In some embodiments, said cleavable cross-linker is a matrix metalloproteinase (MMP)-cleavable peptide. In some embodiments, said peptide comprises an amino acid sequence CGPQGIAGQGCR (SEQ ID NO: 3). Additional molecules suitable for crosslinking the hydrogel are described below.

In some embodiments, said synthetic polymers further comprised alkene containing moieties covalently attached to the polysaccharide polymer chains prior to cross-linking. In some embodiments, the alkene containing moiety is methacrylate, acrylate, or maleimide. Additional moieties suitable as handles for crosslinking the hydrogel are described below.

In some embodiments, the hydrogel further comprises a cell-adhesive peptide. In some embodiments, the cell-adhesive peptide comprises an amino acid sequence RGD. In some embodiments, said cell-adhesive peptide comprises an amino acid sequence CGRGDS (SEQ ID NO: 1). Additional cell-adhesive peptides suitable for incorporation into the hydrogel are described below.

In some embodiments, the hydrogel further comprises at least one bioactive agent, such as a growth factor. In some embodiments, said at least one growth factor is a selected from the group consisting of a vascular endothelial growth factor (VEGF), a fibroblast growth factor (FGF), a bone morphogenic protein (BMP), an epidermal growth factor (EGF), a platelet derived growth factor (PDGF), a WNT, and a combination thereof. In some embodiments, said at least one growth factor is a cytokine, optionally wherein the cytokine is an interleukin, an interferon, or chemokine. Additional bioactive agents suitable for incorporation into the hydrogel are described below.

In some embodiments, the hydrogel further comprises a population of cells. In some embodiments, said population of cells comprises one cell type. In some embodiments, said population of cells comprises two or more cell types. In some embodiments, said population of cells comprises parenchymal cells. In some embodiments, said parenchymal cells are of heart, lung, liver, kidney, adrenal gland, pituitary gland, pancreas, or muscle. In some embodiments, said population of cells comprises stromal cells. In some embodiments, said population of cells comprises endothelial cells. In some embodiments, said population of cells comprises endothelial cells and fibroblasts. Additional populations and types of cells suitable for incorporation into the hydrogel are described below.

In some embodiment, the hydrogel may further comprise an additional polymer that is different from the synthetic polymer of the disclosure. In some embodiments, the hydrogel comprises an additional polymer that is the unmodified polysaccharide corresponding to the synthetic polymer of the disclosure. In some embodiments, the hydrogel comprises an additional polymer, wherein the additional polymer is different from the polysaccharide corresponding to the synthetic polymer of the disclosure. In some embodiments, the additional polymer that may be used to generate the hydrogel may be a polysaccharide, e.g., dextran, alginate, agarose, chondroitin sulfate, chitin/chitosan, cellulose, dextran, starch, and glycogen, galactogen, inulin, pectin, and hyaluronic acid. In other embodiments, the additional polymer that may be used to generate the hydrogel is not a polysaccharide. Exemplary polymers include, but are not limited to, PEG, HEMA, and PHEMA. Additional polymers and methods to make the hydrogels from those polymers may be found in the art.

Bioactive Agent

In some embodiments, the synthetic polymer described herein may be associated with at least one bioactive agent. In other embodiments, the hydrogel described herein comprises at least one bioactive agent. In some embodiments, the bioactive agent is a growth factor. In some embodiments, the growth factor is a cytokine, e.g., an interleukin, an interferon, or chemokine. In some embodiments, the cytokine is an immunomodulatory cytokine. In some embodiments, the bioactive agent is an immunomodulatory agent. In some embodiments, the bioactive agent is an anti-inflammatory agent. In some embodiments, the bioactive agent is an extracellular matrix protein.

Suitable growth factors and cytokines that may be incorporated into the hydrogel include, but are not limited, to stem cell factor (SCF), granulocyte-colony stimulating factor (G-CSF), granulocyte-macrophage stimulating factor (GM-CSF), stromal cell-derived factor-1, steel factor, VEGFs, TGFβ, platelet derived growth factors (PDGFs), angiopoeitins (Ang), epidermal growth factor (EGF), bFGF, HNF, NGF, fibroblast growth factors (FGFs), hepatocye growth factor, liver growth factor (LGF), insulin-like growth factor (IGF-1), interleukin (IL)-3, IL-1α, IL-1β, IL-4, IL-6, IL-7, IL-8, IL-10, IL-11, IL-12, IL-13, IL-18, colony-stimulating factors, thrombopoietin, erythropoietin, fit3-ligand, tumor necrosis factor α (TNFα), IFNγ, a growth factor of the bone morphogenetic protein (BMP) family (e.g. BMP1, BMP2, BMP3, BMP4, BMP5, BMP6, BMP7, BMP8a, BMP8b, BMP10, BMP11, BMP15) and a growth factor that functions in the Wnt signaling pathway (e.g., WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11 and WNT16). Other examples are described in Dijke et al., “Growth Factors for Wound Healing”, Bio/Technology, 7:793-798 (1989); Mulder G D, Haberer Pa., Jeter K F, eds. Clinicians' Pocket Guide to Chronic Wound Repair. 4th ed. Springhouse, Pa.: Springhouse Corporation; 1998:85; Ziegler T. R., Pierce, G. F., and Herndon, D. N., 1997, International Symposium on Growth Factors and Wound Healing: Basic Science & Potential Clinical Applications (Boston, 1995, Serono Symposia USA), Publisher: Springer Verlag, the entire contents of which are incorporated herein.

In some embodiments, the hydrogel comprises a vascular endothelial growth factor (VEGF). VEGF is a key protein in physiological angiogenesis (or neo-vascularization), or formation of new blood vessels. N. Ferrara et al., The biology of VEGF and its receptors, 9 Nat. Med. 669-676 (2003), the entire contents of which are incorporated herein.

In some embodiments, the hydrogel comprises a fibroblast growth factor (FGF).

In some embodiments, the hydrogel comprises an anti-inflammatory agent. Non-limiting examples of a suitable anti-inflammatory agent include corticosteroids, nonsteroidal anti-inflammatory drugs (e.g., aspirin, phenylbutazone, indomethacin, sulindac, tolmetin, ibuprofen, piroxicam, and fenamates), acetaminophen, phenacetin, gold salts, chloroquine, D-Penicillamine, methotrexate colchicine, allopurinol, probenecid, and sulfinpyrazone.

In some embodiments, the hydrogel comprises a bioactive agent selected from angiopoietins, extracellular matrix proteins (e.g., fibronectin, vitronectin, collagen), adhesion proteins, BMPs, TGFbeta, SDFs, interleukins, interferons, CXCLs, and lipoproteins. In some embodiments, the bioactive agent is any protein having a positive charge.

In some embodiments, the bioactive agent improves the function of the hydrogel. For example, in some embodiments, a hydrogel comprising a bioactive agent enhances multicellular sprouting compared to a hydrogel lacking a bioactive agent. In some embodiments, the bioactive agent modifies the function of the hydrogel for various biomedical applications, e.g., regulating inflammatory response and tissue engineering e.g., engineered organoids, liver tissue and bone tissue.

Adherence Material

In some embodiments, the hydrogel comprises an adherence material. The term “adherence material” is a material incorporated into a hydrogel disclosed herein to which a cell or microorganism has some affinity, such as a binding agent. The material can be incorporated, for example, into a hydrogel prior to seeding with parenchymal and/or non-parenchymal cells. The material and a cell or microorganism interact through any means including, for example, electrostatic or hydrophobic interactions, covalent binding or ionic attachment. The material may include, but is not limited to, antibodies, proteins, peptides, nucleic acids, peptide aptamers, nucleic acid aptamers, sugars, proteoglycans, or cellular receptors.

The type of adherence material(s) (e.g., ECM materials, sugars, proteoglycans etc.) will be determined, in part, by the cell type or types to be cultured. ECM molecules found in the parenchymal cell's native microenvironment are useful in maintaining the function of both primary cells, and precursor cells and/or cell lines. For example, hepatocytes are known to bind to collagen. Therefore, collagen is well suited to facilitate binding of hepatocytes. The liver has heterogeneous staining for collagen I, collagen III, collagen IV, laminin, and fibronectin. Hepatocytes also display integrins β1, β2, α1, α2, α5, and the nonintegrin fibronectin receptor Agp110 in vivo. Cultured rat hepatocytes display integrins α1, α3, α5, β1, and α6μ1, and their expression is modulated by the culture conditions.

In some embodiments, the adherence material comprises a cell-adhesive peptide. In some embodiments, the cell-adhesive peptide is an extracellular matrix protein-derived cell-adhesive peptide. In some embodiments, the cell-adhesive peptide is an RGD peptide or comprises the sequence RGD. In some embodiments, the RGD peptide is CGRGDS (SEQ ID NO: 1). In some embodiments, the cell-adhesive peptide comprises the sequence MNYYSNS (SEQ ID NO: 5) or CNYYSNS (SEQ ID NO: 6). In some embodiments, the cell-adhesive peptide comprises the sequence DAPS (SEQ ID NO: 7). In some embodiments, the cell-adhesive peptide comprises the sequence AELDVP (SEQ ID NO: 8) or VALDEP (SEQ ID NO: 9). In some embodiments, the cell-adhesive peptide comprises the sequence GFOGER (SEQ ID NO: 10). In some embodiments, the cell-adhesive peptide comprises the sequence NGRAHA (SEQ ID NO: 11). Other examples of cell-adhesive peptides are described in Huettner et al., “Discovering cell-adhesion peptides in tissue engineering: Beyond RGD” Tissue Engineering, 36(4): 372-383 (2018), the entire content of which is incorporated by reference herein.

In some embodiments, the hydrogel comprises more than one type of cell-adhesive peptide. In some other embodiments, the hydrogel comprises two, three, four, or five different cell-adhesive peptides.

In some embodiments, the hydrogel comprises one or more cell-adhesive peptides at a concentration of 0.1-10 mM, 0.5-10 mM, 1-20 mM, 1-50 mM, 5-100 mM, 5-200 mM, 10-50 mM, 25-75 mM, 10-200 mM, 10-500 mM, 50-100 mM, or 0.1-1M.

In some embodiments, the hydrogel comprises the RGD peptide CGRGDS (SEQ ID NO: 1) at a concentration of 2-100 mM, 20-80 mM, 30-70 mM, or 40-60 mM, e.g., about 50 mM.

In some embodiments, the cell-adhesive peptide is crosslinked to the hydrogel. In some embodiments, the cell-adhesive peptide binds without crosslinking to the hydrogel.

Cells

In some embodiments, a hydrogel provided herein comprises at least one population of cells. In some embodiments, a cell implant provided herein comprises at least one population of cells. In some embodiments, an engineered tissue construct provided herein comprises at least one population of cells. In some embodiments, a composition provided herein comprises a synthetic polymer or hydrogel and at least one population of cells.

In some embodiments, a synthetic polymer of the disclosure is used in combination with a cell implant or an engineered tissue construct comprising at least one population of cells. In some embodiments, a hydrogel of the disclosure is used in combination with a cell implant or an engineered tissue construct comprising at least one population of cells.

In some embodiments, the hydrogel, composition, cell implant and/or engineered tissue construct described herein comprises parenchymal cells. In some embodiments, the hydrogel, composition, cell implant and/or engineered tissue construct described herein comprises non-parenchymal cells, e.g., stromal cells. In some embodiments, the hydrogel, composition, cell implant and/or engineered tissue construct described herein comprises parenchymal and non-parenchymal cells (e.g., stromal cells).

Parenchymal cells can be obtained from a variety of sources including, but not limited to, liver, skin, pancreas, neuronal tissue, muscle (e.g., heart and skeletal), and the like. Parenchymal cells can be obtained from parenchymal tissue using any one of a host of art-described methods for isolating cells from a biological sample, e.g., a human biological sample. Parenchymal cells. e.g., human parenchymal cells, can be obtained by biopsy or from cadaver tissue. In certain embodiments, parenchymal cells are derived from lung, kidney, nerve, heart, fat, bone, muscle, thymus, salivary gland, pancreas, adrenal, spleen, gall bladder, liver, thyroid, parathyroid, small intestine, uterus, ovary, bladder, skin, testes, prostate, pituitary gland, or mammary gland.

In certain embodiments, hydrogels, cell implants and engineered tissue constructs contain human parenchymal cells optimized to maintain the appropriate morphology, phenotype and cellular function conducive to use in the methods of the disclosure. Primary human parenchymal cells can be isolated and/or pre-cultured under conditions optimized to ensure that the parenchymal cells of choice (e.g., hepatocytes) initially have the desired morphology, phenotype and cellular function and, thus, are poised to maintain said morphology, phenotype and/or function in the constructs, and in vivo upon implantation to create the engineered tissue seeds described herein.

Non-parenchymal cells are cells that support parenchymal cells in an organ. Non-parenchymal cells include, e.g., stromal cells (e.g., stem cell) such as endothelial cells and fibroblasts

Cells useful in the constructs and methods of the disclosure are available from a number of sources including commercial sources. For example, hepatocytes may be isolated by conventional methods (Berry and Friend, 1969, J. Cell Biol. 43:506-520) which can be adapted for human liver biopsy or autopsy material. In general, cells may be obtained by perfusion methods or other methods known in the art, such as those described in U.S. Pat. Pub. No. 20060270032.

Parenchymal and non-parenchymal cell types that can be used include, but are not limited to, hepatocytes, pancreatic cells (alpha, beta, gamma, delta), myocytes, enterocytes, renal epithelial cells and other kidney cells, brain cell (neurons, astrocytes, glia), respiratory epithelium, stem cells, and blood cells (e.g., erythrocytes and lymphocytes), adult and embryonic stem cells, blood-brain barrier cells, and other parenchymal cell types known in the art, fibroblasts, endothelial cells, and other non-parenchymal cell types known in the art.

In some embodiments, the cells are mammalian cells, although the cells may be from two different species (e.g., humans, mice, rats, primates, pigs, and the like). The cells can be primary cells, or they may be derived from an established cell-line. Cells can be from multiple donor types, can be progenitor cells (e.g., liver progenitor cells), tumor cells, and the like. In some embodiments, the cells are freshly isolated cells (for example, encapsulated within 24 hours of isolation), e.g., freshly isolated hepatocytes from cadaveric donor livers. Although any combination of cell types that promotes maintenance of differentiated function of the parenchymal cells can be used (e.g., parenchymal and one or more populations of non-parenchymal cells, e.g., stromal cells), an exemplary combination of cells for producing the constructs include, without limitation: fibroblasts and endothelial cells. Other exemplary combinations include, without limitation, (a) human hepatocytes (e.g., primary hepatocytes) and fibroblasts (e.g., normal or transformed fibroblasts, including, for example, non-human transformed fibroblasts); (b) hepatocytes and at least one other cell type, particularly liver cells, such as Kupffer cells, Ito cells, endothelial cells, and biliary ductal cells; and (c) stem cells (e.g., liver progenitor cells, oval cells, hematopoietic stem cells, embryonic stem cells, and the like) and a non-parenchymal cell population, for example, stromal cells (e.g., fibroblasts). In some embodiments it may be desirable to include immune cells in the constructs, e.g., Kupffer cells, macrophages, B-cells, dendritic cells, etc.

Hepatocytes may be from any source known in the art, e.g., primary hepatocytes, progenitor-derived, ES-derived, induced pluripotent stem cells (iPS-derived), etc. Hepatocytes useful in the constructs and methods described herein may be produced by the methods described in Takashi Aoi et al., Science 321 (5889): 699-702; U.S. Pat. Nos. 5,030,105; 4,914,032; 6,017,760; 5,112,757; 6,506,574; 7,186,553; 5,521,076; 5,942,436; 5,580,776; 6,458,589; 5,532,156; 5,869,243; 5,529,920; 6,136,600; 5,665,589; 5,759,765; 6,004,810; U.S. patent application Ser. Nos. 11/663,091; 11/334,392; 11/732,797; 10/810,311; and PCT application PCT/JP2006/306783, all of which are incorporated herein by reference in their entirety.

Further cell types which may be cultured include pancreatic cells (alpha, beta, gamma, delta), enterocytes, renal epithelial cells, astrocytes, muscle cells, brain cells, neurons, glia cells, respiratory epithelial cells, lymphocytes, erythrocytes, blood-brain barrier cells, kidney cells, cancer cells, normal or transformed fibroblasts, liver progenitor cells, oval cells, adipocytes, osteoblasts, osteoclasts, myoblasts, beta-pancreatic islets cells, stem cells (e.g., embryonic stem cells, hematopoietic stem cells, mesenchymal stem cells, endothelial stem cells, etc.), cells described in U.S. patent application Ser. No. 10/547,057 paragraphs 0066-0075 which is incorporated herein by reference, myocytes, keratinocytes, and indeed any cell type that adheres to a substrate.

In some embodiments, the hydrogel, cell implant and/or engineered tissue construct comprises endothelial cells. In some embodiments, the endothelial cells are adult vein endothelial cells, adult artery endothelial cells, embryonic stem cell-derived endothelial cells, iPS-derived endothelial cells, umbilical vein endothelial cells, umbilical artery endothelial cells, endothelial progenitors cells derived from bone marrow, endothelial progenitors cells derived from cord blood, endothelial progenitors cells derived from peripheral blood, endothelial progenitors cells derived from adipose tissues, endothelial cells derived from adult skin, or a combination thereof. In some embodiments, the umbilical vein endothelial cells are human umbilical vein endothelial cells (HUVEC).

In some embodiments, the hydrogel, cell implant and/or engineered tissue construct comprises fibroblast and/or fibroblast-like cells. In some embodiments, the fibroblasts are human foreskin fibroblasts, human embryonic fibroblasts, mouse embryonic fibroblasts, skin fibroblasts cells, vascular fibroblast cells, myofibroblasts, smooth muscle cells, mesenchymal stem cells (MSCs)-derived fibroblast cells, or a combination thereof. In some embodiments the fibroblasts are normal human dermal fibroblasts (NHDFs).

Methods for Generating Hydrogels and Hydrogel Properties

In some embodiments, the disclosure provides a method for generating a hydrogel comprising a synthetic heparin mimetic described herein. In some embodiments, a synthetic polymer of the disclosure, e.g., a heparin mimetic, is modified to comprise a moiety that can facilitate crosslinking of the polymer to generate a hydrogel. In some embodiments, the moiety comprises an alkyne groups and may be selected from the group consisting of methacrylate, acrylate, maleimide and vinyl sulfone. In some embodiments, the moiety is methacrylate. In some embodiments, a plurality of synthetic polymers, e.g., synthetic heparin mimetics, comprising methacrylate are cross-linked.

In some embodiments, a synthetic polymer of the disclosure may be crosslinked to form a hydrogel by any methods known in the art for preparing hydrogels.

Polymers for use herein are preferably crosslinked, for example, ionically crosslinked. In some embodiments, the methods and constructs described herein use polymers in which polymerization can be promoted photochemically (i.e., photocrosslinked), by exposure to an appropriate wavelength of light (i.e., photopolymerizable) or a polymer which is weakened or rendered soluble by light exposure or other stimulus. Although some of the polymers listed above are not inherently light sensitive (e.g. collagen, HA), they may be made light sensitive by the addition of acrylate or other photosensitive groups.

In some embodiments, the method utilizes a photoinitiator. A photoinitiator is a molecule that is capable of promoting polymerization of hydrogels upon exposure to an appropriate wavelength of light as defined by the reactive groups on the molecule. In the context of the disclosure, photoinitiators are cytocompatible. A number of photoinitiators are known that can be used with different wavelengths of light. For example, 2,2-dimethoxy-2-phenyl-acetophenone, HPK 1-hydroxycyclohexyl-phenyl ketone and Irgacure 2959 (hydroxyl-1-[4-(hydroxyethoxy)phenyl]-2methyl-lpropanone) are all activated with UV light (365 nm). Other crosslinking agents activated by wavelengths of light that are cytocompatible (e.g. blue light) can also be used with the methods described herein.

In some embodiments, the method involves the use of polymers bearing non-photochemically polymerizable moieties. In some embodiments, the non-photochemically polymerizable moieties are Michael acceptors. Non-limiting examples of such Michael acceptor moieties include α,β-unsaturated ketones, esters, amides, sulfones, sulfoxides, phosphonates. Additional non-limiting examples of Michael acceptors include quinines and vinyl pyridines. In some embodiments, the polymerization of Michael acceptors is promoted by a nucleophile. Suitable nucleophiles include, but are not limited to thiols, amines, alcohols and molecules possessing thiol, amine and alcohol moieties. In some embodiments, the disclosure features use of thermally crosslinked polymers.

In some embodiments, the hydrogel is non-covalently cross-linked.

In some other embodiments, the hydrogel is covalently cross-linked. In some embodiments, the cross-linker is a peptide cross-linker.

In some embodiments, the hydrogel is cross-linked by a cleavable crosslinker. In some preferred embodiments, the hydrogel is cross-linked by a crosslinker cleavable by a proteinase of a cell. In some embodiments, the cleavable cross-linker comprises a matrix metalloproteinase (MMP)-cleavable peptides.

The MMP-cleavable peptides may be derived from naturally existing protein or artificially designed. In some embodiments, the MMP-cleavable peptide is, e.g., GPQGIAGQ (SEQ ID NO: 12), GPQGIWGQ (SEQ ID NO: 13), VPMSMRGG (SEQ ID NO: 14), QPQGLAK (SEQ ID NO: 15), GPLGLSLGK (SEQ ID NO: 16), or GPLGMHGK (SEQ ID NO: 17). Additional MMP-cleavable peptides may be found in Tu, Y. and Zhu, L. “Matrix metalloproteinase-sensitive nanocarriers” Smart Pharmaceutical Nanocarriers; 83-116 (2016), the entire contents of which are incorporated by reference herein.

In some embodiments, the hydrogel is about 0-100% crosslinked, e.g., about 1%, 5%, 10%, 15%, 20%, 25%, 30%, 35%, 40%, 45%, 50%, 60%, 70%, 80%, 90%, or more crosslinked.

In some embodiments, the hydrogel is degradable. In some embodiment, the hydrogel is partially degradable, e.g., 95% 90%, 80%, 60%, 50%, 40%, 30%, 20%, 10%, or 5% degradable.

In some embodiment, the hydrogel is not degradable.

In some embodiments, the hydrogel comprises a stiffness of 1 Pa-1000 kPa, e.g., 1-10,000 Pa, 10-1000 Pa, 10-500 Pa, 10-100 Pa, 0.1-10 kPa, 0.1-20 kPa, 0.1-500 kPa, 1-1000 kPa, 1-500 kPa, 5-1000 kPa, 5-500 kPa, 0.1-500 kPa, 0.1-100 kPa, 1-500 kPa, 1-100 kPa, 5-500 kPa, 5-100 kPa, or 1-50 kPa, e.g., about 1, 2, 5, 10, 15, or 20 kPa. In some embodiments the hydrogel comprises a stiffness of 200-5000 Pa, 500-4000 Pa, 1000-3000 Pa, or 1500-2500 Pa, e.g., about 2000 Pa.

Cells may be patterned within the hydrogel by selective polymerization of the biopolymer or by patterning of the cells using an electrical field or both. In some embodiments, patterned cells suitable for the constructs and methods described herein are localized in specked locations that may occur in repeating structures within 3-dimensional biopolymer rather than being randomly localized throughout 3-dimensional slab of biopolymer, on the surface of a regularly or irregularly shaped 3-dimensional scaffold, or patterned on a 2-dimensional support (e.g. on a glass slide). The cells can be patterned by locating the cells within specific regions of relatively homogeneous slabs of biopolymers (resolution up to about 5 microns) or by creating patterned biopolymer scaffolds of defined patterns wherein the living cells are contained within the hydrogel (resolution up to about 100 microns). Patterning is performed without direct, mechanical manipulation or physical contact and without relying on active cellular processes such as adhesion of the cells.

Relatively homogeneous slab of biopolymer refers to a polymerized biopolymer scaffold that is approximately the same thickness throughout and is essentially the same shape of the casting or DEP chamber in which it was polymerized.

Patterned biopolymer scaffold refers to a biopolymer scaffold that is of a substantially different shape than the casting or DEP chamber in which it was polymerized. The pattern could be in the form of shapes (e.g. circles, stars, triangles) or a mesh or other form. In some embodiments, the biopolymer is patterned to mimic in vivo tissue architecture, such as branching structures.

The methods described herein can be used for the production of any of a number of patterns in single or multiple layers including geometric shapes or a repeating series of dots with the features in various sizes. Alternatively, multilayer biopolymer gels can be generated using a single mask turned in various orientations. The formation of high resolution patterned cells in 3-dimensions can be achieved by methods other than photopolymerization, such that the limitations of the method are overcome.

Stereolithography via photopatterning may be used to introduce perfusion channels, thus significantly improving diffusive transport of oxygen and nutrients to photoencapsulated hepatocytes. In some embodiments, the perfusion channel consists of a single-layer hexagonal branching pattern.

Cells may be patterned within the hydrogel by selective polymerization of the biopolymer or by patterning of the cells using an electrical field or both. Theoretically a single cell can be patterned by locating it in a specific position within a biopolymer; however, in some embodiments a plurality of cells, at least 10, at least 20, at least 100, at least 500 cells, are patterned. Patterning does not require localization of all cells to a single, discrete location within the biopolymer. Cells can be localized, in lines one or two or many cells wide, or in multiple small clusters throughout a relatively homogeneous biopolymer scaffold (e.g. approximately 20,000 clusters of 10 cells each in a single scaffold). The 3-dimensional patterning can also include patterning of cells or other particles in a single plane by DEP as the cells are contained in a three dimensional scaffold. The cell patterning methods described herein, can also be used for patterning of organelles, liposomes, beads and other particles.

Cell organization can be controlled by photopatterning of the hydrogel structure. The photopolymerizable nature of acrylate-based hydrogels enables the adaptation of photolithographic techniques to generate patterned hydrogel networks. In this process, patterned masks printed on transparencies act to localize the UV exposure of the prepolymer solution, and thus, dictate the structure of the resultant hydrogel.

Dielectrophoresis (DEP) can be used alone for patterning of cells in relatively homogeneous slabs of hydrogel or in conjunction with the photopolymerization method. The methods allow for the formation of three dimensional scaffolds from hundreds of microns to tens of centimeters in length and width, and tens of microns to hundreds of microns in height. A resolution of up to 100 microns in the photopolymerization method and possible single cell resolution (10 micron) in the DEP method is achievable. Photopolymerization apparatus, DEP apparatus, and other methods to produce 3-dimensional co-cultures are described in U.S. patent application Ser. No. 11/035,394, which is incorporated herein by reference.

In some embodiments, the biopolymers may additionally contain any of a number of growth factors, adhesion molecules, degradation sites or bioactive agents to enhance cell viability or for any of a number of other reasons. Such molecules are well known to those skilled in the art and described herein.

The tunability of scaffold chemistry allows manipulation of cell-matrix interactions of encapsulated human hepatocytes in vitro. NHS ester chemistry may be used to conjugate RGDS (SEQ ID NO: 18), or the negative control RGES (SEQ ID NO: 19) peptide, to acrylate polymer monomers. In some embodiments, the RGDS (SEQ ID NO: 18) peptide is covalently attached to a component of the hydrogel. In some embodiments, the RGDS (SEQ ID NO: 18) peptide is covalently attached to an acrylate PEG monomer polymerized in the hydrogel. ECM-derived peptides can be included, for example, at a concentration of about 1-100 μM/ml, for example, at a concentration of about 2-50 μM/ml or about 5-20 μM/ml. In some embodiments, incorporation of said functionalized monomers within the hydrogel network improves encapsulated cells synthetic and secretory functions by two- to three-fold compared to RGES (SEQ ID NO: 19) controls cultured over one week in vitro. Other conjugation chemistries are well-known in the art and interchangeable with the NHS chemistries exemplified herein.

The hydrogel may be polymerized homogeneously or through a mask to result in selective photopolymerization and patterning of the biopolymer. In some embodiments, other ways of photopatterning are used including, but not limited to, shining light through an emulsion mask, and also including shining light in a pattern through a digital pattern generator or scanning a laser in a pattern as in stereolithography or using a hologram. In certain embodiments of the above methods, the hydrogel comprises perfusion channels supporting diffusive transport of oxygen and/or nutrients. In some embodiments of the above methods, the scaffold is biodegradable. Photopatterning allows thicker constructs of to be utilized due to increased nutrient and/or oxygen transport to encapsulated cells.

Soluble factors can be included at about 1-1000 ng/ml and, in some embodiments, can be included at up to, for example, 100 μg/ml. Soluble factors can be added or released (e.g., drug delivery means) or can be secreted by supporting cells to achieve the desired concentration, for example, at a specified time after encapsulation or implantation.

### III. Uses of Synthetic Polymers

In some embodiments, the disclosure provides methods for using the synthetic polymers, e.g., synthetic heparin mimetics, and hydrogels described herein.

The synthetic polymer of the invention comprises an amount of negative charge that, in some embodiments, is similar to the amount of negative charge present in heparin. Accordingly, the synthetic polymer of the disclosure can mimic the functional properties of heparin. For example, the synthetic polymer of the disclosure has the potential to bind various bioactive agents, e.g., growth factors, that naturally bind to heparin, or which are highly positively charged. Therefore, the synthetic polymer of the disclosure, as well as the hydrogel comprising the synthetic polymer described herein can bind various bioactive agents, e.g., growth factors, thereby preventing the bioactive agents from diffusing away and maintaining the bioactive agents at a high concentration locally, so that they can act on cells and promote various cell functions.

Exemplary growth factors and cytokines that may bind to the synthetic polymer of the disclosure, or that may be administered in combination with the synthetic polymer of the disclosure may be selected from the group consisting of stem cell factor (SCF), granulocyte-colony stimulating factor (G-CSF), granulocyte-macrophage stimulating factor (GM-CSF), stromal cell-derived factor-1, steel factor, VEGF, TGFβ, platelet derived growth factor (PDGF), angiopoetins (Ang), epidermal growth factor (EGF), bFGF, HNF, NGF, fibroblast growth factor (FGF), hepatocye growth factor, liver growth factor (LGF) insulin-like growth factor (IGF-1), interleukin (IL)-3, IL-1α, IL-1β, IL-6, IL-7, IL-8, IL-11, and IL-13, colony-stimulating factors, thrombopoietin, erythropoietin, fit3-ligand, tumor necrosis factor α (TNFα), a growth factor of the bone morphogenetic protein (BMP) family (e.g. BMP1, BMP2, BMP3, BMP4, BMP5, BMP6, BMP7, BMP8a, BMP8b, BMP10, BMP11, BMP15), a growth factor that functions in the Wnt signaling pathway (e.g., WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11 and WNT16), and other growth factors and cytokines known in the art.

In some embodiments, a synthetic polymer, a composition comprising the synthetic polymer or a hydrogel comprising the synthetic polymer as described herein may be administered in the absence of cells. In other embodiments, synthetic polymer, a composition comprising the synthetic polymer or a hydrogel comprising the synthetic polymer as described herein may be administered in combination with one or more populations of cells.

In some embodiments, the synthetic polymer of the disclosure may be administered to subject, optionally with one or more bioactive agent, e.g., growth factor or cytokine, as a part of a composition that is not a hydrogel. Such composition may be, for example, a liquid composition comprising the synthetic polymer of the disclosure and a buffer, e.g., a phosphate buffer.

In some embodiments, the synthetic polymer of the disclosure may be administered to subject, optionally with one or more bioactive agent, e.g., growth factor or cytokine, as a part of a composition that is a hydrogel.

In some embodiments, a synthetic polymer of the disclosure, e.g., synthetic heparin mimetic, can be administered to a subject alone or in combination with one or more populations of cells. In some embodiments, a hydrogel comprising a synthetic polymer, e.g., synthetic heparin mimetic, can be administered to a subject alone or in combination with one or more population of cells. In some embodiments, the synthetic polymer, e.g., synthetic heparin mimetic, or hydrogel is administered sequentially or simultaneously with one or more population of cells.

In some embodiments, a synthetic polymer, e.g., synthetic heparin mimetic, or hydrogel enhances or promotes vascularization, survival, and/or engraftment of an engineered tissue construct implanted in a subject compared to an engineered tissue construct implanted in a subject without the synthetic polymer, e.g., heparin mimetic, or hydrogel. After a synthetic polymer of the disclosure, is administered to a subject, the synthetic polymer or a hydrogel comprising a synthetic polymer may associate with the native extracellular matrix of the subject and bind one or more bioactive agents e.g., growth factors and cytokines, that are present in the subject or that are produced by cells in the tissue of the subject. Therefore, administration of the synthetic polymer of the disclosure can promote natural processes, including but not limited to cell proliferation, cell differentiation, vascularization and wound healing.

In some embodiments, the disclosure provides a method of promoting vascularization in a subject, e.g., vascularization of diseased or damaged tissue in a subject, comprises administering to the subject the synthetic polymer, or a hydrogel comprising the synthetic polymer, or a composition comprising the synthetic polymer as described herein.

In some embodiments, promoting vascularization in a diseased tissue results in a higher vascularization in a diseased tissue than vascularization in a diseased tissue achieved using a corresponding polysaccharide in which hydroxyl groups have not been modified by converting the hydroxyl groups into negatively charged functional groups or using a hydrogel comprising said corresponding polysaccharide. In some embodiments, the diseased tissue comprises a region of ischemia.

In some embodiments, the disclosure provides a method for inducing or enhancing vascularization, e.g., angiogenesis, in a subject, e.g., in a diseased tissue, e.g., an ischemic tissue. Angiogenesis is a complex multi-step process involving endothelial cell activation, controlled proteolytic degradation of the extracellular matrix (ECM), proliferation and migration of endothelial cells, and formation of capillary vessel lumina. Diaz-Flores et al., 33 Anat. Histol. Embryol. 334-338 (2004).

In some embodiments, the synthetic polymer of the invention may bind one or more growth factors that promote vascularization, e.g., VEGF. In some embodiments, the synthetic polymer of the disclosure may be administered in combination with one or more growth factors that promote vascularization, e.g., VEGF. In some embodiments, administration of the synthetic polymer to a tissue of a subject may promote vascularization in the tissue.

In some embodiments, the present disclosure provides a method for promoting, inducing or enhancing wound healing and/or tissue regeneration that comprises administering to a subject in need thereof a synthetic polymer, or a hydrogel comprising the synthetic polymer, or a composition comprising the synthetic polymer described herein in combination with one or more growth factors that promote and regulate cell proliferation and/or cell differentiation. Wound healing and tissue regeneration are processes that requires cell proliferation, cell remodeling and vascularization of the new tissue. In some embodiments, the synthetic polymer of the invention may bind one or more growth factors that promote and regulate cell proliferation and/or cell differentiation, e.g., PDGF, FGF, EGF, IGF-I, IF-II, TGF-α, TGF-β, growth factors of the BMP family and growth factors of the Wnt family, and other growth factors known in the art. In some embodiments, the wound is a diabetic ulcer.

In some embodiments, the present disclosure provides a method for modulating an immune response that comprises administering to a subject in need thereof a synthetic polymer, or a hydrogel comprising the synthetic polymer, or a composition comprising the synthetic polymer described herein in combination with a cytokine. The synthetic polymer of the invention may bind one or more cytokines that function to modulate an immune response at the site of administration of the synthetic polymer. Exemplary cytokines that may be administered in combination with a synthetic polymer of the disclosure include, e.g., cytokines in the interleukin (IL) family (e.g., IL-3, IL-1α, IL-1β, IL-6, IL-7, IL-8, IL-11, IL-13), cytokines in the tumor necrosis factor (TNF) family (e.g., TNF-α) and other immunomodulatory cytokines known in the art.

In some embodiments, the synthetic polymer, a hydrogel comprising the synthetic polymer, or a composition comprising the synthetic polymer, may be administered in combination with one or more cytokines that suppresses an immune response, e.g., to prevent rejection of an implant. In some embodiments, the synthetic polymer, a hydrogel comprising the synthetic polymer, or a composition comprising the synthetic polymer, is administered in combination with one or more cytokines that promotes bone marrow regrowth and regeneration.

In some embodiments, the present disclosure provides a method for promoting bone or cartilage formation that comprises administering to a subject in need thereof a synthetic polymer, or a hydrogel comprising the synthetic polymer, or a composition comprising the synthetic polymer in combination with a bone morphogenic protein (BMP).

In some embodiments, the present disclosure provides a method for promoting tissue regeneration that comprises administering to a subject in need thereof a synthetic polymer, or a hydrogel comprising the synthetic polymer, or a composition comprising the synthetic polymer in combination with a growth factor of the Wnt family.

In certain aspect, the synthetic polymer may be administered in a composition comprising one or more bioactive agent, e.g., growth factor, cytokine, or a combination thereof, to a subject. In some embodiments, the synthetic polymer of the invention may be incubated with one or more bioactive agent, e.g., growth factor and/or cytokine, before the synthetic polymer is administered to a subject.

In another aspect, the disclosure provides a method of promoting activity of a growth factor in a subject, comprising administering to a subject the growth factor in combination with the synthetic polymer, a composition comprising the synthetic polymer, or the hydrogel comprising the synthetic polymer, such that the growth factor activity is promoted. Exemplary growth factors and cytokines may be selected from the group consisting of stem cell factor (SCF), granulocyte-colony stimulating factor (G-CSF), granulocyte-macrophage stimulating factor (GM-CSF), stromal cell-derived factor-1, steel factor, VEGF, TGFβ, platelet derived growth factor (PDGF), angiopoetins (Ang), epidermal growth factor (EGF), bFGF, HNF, NGF, fibroblast growth factor (FGF), hepatocye growth factor, liver growth factor (LGF) insulin-like growth factor (IGF-1), interleukin (IL)-3, IL-1α, IL-1β, IL-6, IL-7, IL-8, IL-11, and IL-13, colony-stimulating factors, thrombopoietin, erythropoietin, fit3-ligand, tumor necrosis factor α (TNFα), a growth factor of the bone morphogenetic protein (BMP) family (e.g. BMP1, BMP2, BMP3, BMP4, BMP5, BMP6, BMP7, BMP8a, BMP8b, BMP10, BMP11, BMP15), a growth factor that functions in the Wnt signaling pathway (e.g., WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11 and WNT16), and other growth factors and cytokines known in the art.

In certain embodiments, the synthetic polymer and hydrogel described herein can be administered, e.g., injected or implanted in a subject. Subjects that can be administered a synthetic polymer, a composition comprising the synthetic polymer, or a hydrogel comprising the synthetic polymer may be human subject or non-human subject. Non-limiting examples of non-human subjects include non-human primates, dogs, cats, mice, rats, guinea pigs, rabbits, fowl, pigs, horses, cows, goats, sheep, etc. In certain embodiments, the subject can be any animal. In certain embodiments, the subject can be any mammal. In certain embodiments, the subject is a human.

In certain embodiments, the synthetic polymer, a composition comprising the synthetic polymer, or a hydrogel comprising the synthetic polymer, may be administered to a subject by injection. In other embodiments, the synthetic polymer, a composition comprising the synthetic polymer or a hydrogel comprising the synthetic polymer may be administered to a subject by implantation into a tissue in a subject. Non-limiting examples of tissues include connective tissue (e.g., cartilage, bone, areolar tissue, adipose tissue, reticular tissue, tendon), epithelial tissue, muscle tissue, and nervous tissue. In some embodiments, the tissue may be in an organ, e.g., heart, lung, liver, kidney, muscles, pancreas, adrenal gland or pituitary gland of the subject. In some embodiments, the tissue may be a diseased or damaged tissue that may require regeneration and/or re-vascularization, e.g., a tissue comprising ischemia.

Use of the Synthetic Polymer for Cell Implants

In some embodiments, the present disclosure also provides a method of promoting vascularization of a cell implant or an engineered tissue construct in a subject that comprises administering to the subject the cell implant or the engineered tissue construct in combination with a synthetic polymer, a composition comprising the synthetic polymer, or a hydrogel comprising the synthetic polymer of the disclosure.

In some embodiments, promoting vascularization of a cell implant or an engineered tissue construct results in an amount of vascularization of an engineered tissue construct that is greater, e.g., at least 10% greater, at least 25% greater, at least 50% greater, at least 75% greater or at least 100% greater than the amount of vascularization of an engineered tissue construct obtained using a corresponding polysaccharide in which hydroxyl groups have not been modified by converting the hydroxyl groups into negatively charged functional groups or using a composition or a hydrogel comprising the corresponding polysaccharide.

In some embodiments, the present disclosure provides a method of promoting cell survival in a cell implant or an engineered tissue construct in a subject, comprising administering to the subject the cell implant or engineered tissue construct in combination with the synthetic polymer, a composition comprising the synthetic polymer of a hydrogel comprising the synthetic polymer of the disclosure. In some embodiments, promoting cell survival in a cell implant or an engineered tissue construct results in a greater cell survival in a cell implant or an engineered tissue construct than cell survival in a cell implant or an engineered tissue construct achieved using a corresponding polysaccharide in which hydroxyl groups have not been modified by converting the hydroxyl groups into negatively charged functional groups or using a composition or a hydrogel comprising the corresponding polysaccharide. In some embodiments, promoting cell survival encompasses preventing death of cells in a cell implant or an engineered tissue construct to support proper functioning of the cell implant or an engineering tissue construct after administration to a subject.

Some embodiments, the present disclosure also provides a method of promoting engraftment of a cell implant or an engineered tissue construct in a subject, comprising administering to the subject the cell implant or engineered tissue construct in combination with the synthetic polymer, the composition comprising the synthetic polymer or the hydrogel comprising the synthetic polymer described herein. In some embodiments, promoting engraftment of a cell implant or an engineered tissue construct results in a greater engraftment of a cell implant or an engineered tissue construct than engraftment of a cell implant or an engineered tissue construct achieved using a corresponding polysaccharide in which hydroxyl groups have not been modified by converting the hydroxyl groups into negatively charged functional groups or using a composition or a hydrogel comprising the corresponding polysaccharide.

In some embodiments, a synthetic polymer, a composition comprising a synthetic polymer, or a hydrogel comprising the synthetic polymer of the disclosure may be administered in combination with one or more population of cells.

In some embodiments, a cell population that may be administered in combination with the synthetic polymer, or a composition comprising the synthetic polymer or hydrogel comprising the synthetic polymer may be parenchymal cells. In some embodiments, the parenchymal cells are derived from, e.g., lung, kidney, nerve, heart, fat, bone, muscle, thymus, salivary gland, pancreas, adrenal, spleen, gall bladder, liver, thyroid, parathyroid, small intestine, uterus, ovary, bladder, skin, testes, prostate, or mammary gland.

In some embodiments, a cell population that may be administered in a combination with a synthetic polymer, or a composition comprising the synthetic polymer, or a hydrogel comprising the synthetic polymer of the disclosure may be non-parenchymal cells (e.g., endothelial cells, stromal cells, Kupffer cells, stellate cells).

Parenchymal and non-parenchymal cell types that can be used include, but are not limited to, hepatocytes, pancreatic cells (alpha, beta, gamma, delta), myocytes, enterocytes, renal epithelial cells and other kidney cells, osteoclast, brain cell (neurons, astrocytes, glia), respiratory epithelium, stem cells, adult and embryonic stem cells, blood-brain barrier cells, and other parenchymal cell types known in the art, fibroblasts, endothelial cells, and other non-parenchymal cell types known in the art.

The cells for administration with the synthetic polymer of the disclosure are selected based on the necessary tissue functionality and structures required to replace or facilitate the repair of a tissue of the subject. For examples, the cells selected for administration with the synthetic polymer maybe muscle cells to provide contractile structures, vascular and/or neural cells to provide conductive elements, metabolically active secretory cells, such as liver cells, hormone synthesizing cells, sebaceous cells, pancreatic islet cells or adrenal cortex cells to provide secretory structures, stem cells, such as bone marrow-derived or embryonic stem cells, dermal fibroblasts, skin keratinocytes, Schwann cells for nerve implants, smooth muscle cells and endothelial cells for vessel structures, urothelial and smooth muscle cells for bladder/urethra structures and osteocytes, chondrocytes, and tendon cells for bone and tendon structures, or a combination thereof.

In certain embodiments, the cells may be derived from a subject different from the subject that will receive the administration of the synthetic polymer and cells (e.g., xenograft). In certain embodiments, the cells may be derived from the same subject that will receive the administration of the synthetic polymer and cells (e.g., autograft).

In certain embodiments, the synthetic polymer, a composition comprising the synthetic polymer, or a hydrogel comprising the synthetic polymer and one or more population of cells may be administered, e.g., injected or implanted, in a subject. A subject may be a human subject or a non-human subject. Non-limiting examples of non-human subjects include non-human primates, dogs, cats, mice, rats, guinea pigs, rabbits, fowl, pigs, horses, cows, goats, sheep, etc. In certain embodiments, the subject can be any animal. In certain embodiments, the subject can be any mammal. In certain embodiments, the subject can be a human.

In certain embodiments, the synthetic polymer, a composition comprising the synthetic polymer, or a hydrogel comprising the synthetic polymer and one or more population of cells may be administered, e.g., injected or implanted, into a tissue in a subject. Non-limiting examples of tissues include connective tissue (e.g., cartilage, bone, areolar tissue, adipose tissue, reticular tissue, tendon), epithelial tissue, muscle tissue, and nervous tissue. In some embodiments, the tissue may be in an organ, e.g., heart, lung, liver, kidney, muscles, pancreas, adrenal gland and pituitary gland, and other organ known in the art. In some embodiments, the tissue may be a diseased or damaged tissue that requires regeneration and/or re-vascularization.

Additionally, in certain aspect, the disclosure provides method of promoting a growth factor-dependent cell therapy that comprises administering to a subject the growth factor-dependent cell therapy in combination with a synthetic polymer, a composition comprising the synthetic polymer or the hydrogel comprising the synthetic polymer as described herein, such that the growth factor-dependent cell therapy is promoted.

In some embodiments, the synthetic polymer may be administered with one or more populations of cells and one or more bioactive agents, e.g., a growth factor or cytokine.

In some embodiments, the growth factor that may be administered with the synthetic polymer and population of cells promotes vascularization, e.g., VEGF, and others known in the art. In some embodiments, the growth factor that may be administered in combination with the synthetic polymer and population of cells promotes survival, proliferation, and/or differentiation of cells, e.g., PDGF, FGF, EGF, IGF-I, IF-II, TGF-α, TGF-β, growth factors of the BMP family and growth factors of the Wnt family, and others known in the art.

In some embodiments, the cytokine that may be administered in combination with the synthetic polymer, a composition comprising the synthetic polymer or a hydrogel comprising the synthetic polymer and population of cells is a cytokine. In a preferred embodiments, the synthetic polymer is administered with one or more cytokine that suppress an immune response, e.g., to prevent rejection of a cell implant.

In certain embodiments, the one or more populations of cells are part of a tissue.

In one aspect, the disclosure provides a method of promoting vascularization of a tissue graft in a subject that comprises contacting a tissue to be grafted with a synthetic polymer, a composition comprising the synthetic polymer or the hydrogel comprising the synthetic polymer as described herein prior to grafting of the tissue, e.g., ex vivo. In some embodiments, the tissue to be grafted is contacted with the synthetic polymer, a composition comprising the synthetic polymer or the hydrogel comprising the synthetic polymer for an period of time that is sufficient to promote vascularization of the tissue graft upon grafting in the subject.

In some embodiment, a tissue to be grafted may be contacted with the synthetic polymer, a composition comprising the synthetic polymer or the hydrogel comprising the synthetic polymer and one or more bioactive agents, e.g., a growth factor and/or a cytokine, prior to implantation into a subject.

In some embodiments, the synthetic polymer of the disclosure may be administered in combination with one or more population of cells as a part of a composition that is not a hydrogel.

In some embodiments, a synthetic polymer of the disclosure may be administered to a subject in combination one or more populations of cells, optionally with one or more bioactive agents, e.g., growth factor and/or a cytokine, as a part of a hydrogel. In some embodiments, the hydrogel comprising the synthetic polymer and one or more populations of cells, optionally with one or more bioactive agents, is used to generate a scaffold to support survival and function of one or more population of cells, e.g., hepatocytes, as described herein. In some embodiments, the hydrogel can encapsulate cells of one or more distinct cell types. For example, the hydrogel scaffold can include, but is not limited to, muscle cells to provide contractile structures, vascular and/or neural cells to provide conductive elements, metabolically active secretory cells, such as liver cells, hormone synthesizing cells, sebaceous cells, pancreatic islet cells or adrenal cortex cells to provide secretory structures, stem cells, such as bone marrow-derived or embryonic stem cells, dermal fibroblasts, skin keratinocytes, Schwann cells for nerve implants, smooth muscle cells and endothelial cells for vessel structures, urothelial and smooth muscle cells for bladder/urethra structures and osteocytes, chondrocytes, and tendon cells for bone and tendon structures, or a combination thereof. In certain embodiments, the hydrogel scaffold can include other cell types including, but not limited, to endothelial cells and hepatocytes.

Use of the Synthetic Polymer and Hydrogel for Engineered Tissue Construct

In certain embodiments, the hydrogel described herein can be used to generate an engineered tissue construct, e.g., a pre-vascularized tissue graft, for rapid in vivo integration of the engineered tissue construct into the subject receiving the implantation.

Accordingly, in some embodiments, the present disclosure provides an engineered tissue construct comprising the synthetic polymer of the disclosure. As used herein, the term “tissue construct” refers to a construct that comprises cells associated with, e.g., placed on or within, matrices. In some embodiments, an engineered tissue construct may comprise a synthetic polymer, a composition comprising a synthetic polymer or a hydrogel comprising a synthetic polymer of the disclosure and one or more cell populations. In some embodiments, cells may be cultured with the matrix to form a tissue graft. In some embodiments, cells may be cultured with the matrix and a synthetic polymer of the disclosure, a composition comprising a synthetic polymer of the disclosure, or a hydrogel comprising a synthetic polymer of the disclosure to form a tissue construct. In some embodiments, the tissue construct may further comprise one or more growth factors and/or one or more cytokines described herein. In some embodiments, the tissue construct may be a pre-vascularized tissue construct.

In some embodiments, endothelial cells, and optionally fibroblasts, are cultured in the hydrogel of the disclosure to form a vascular network, and the vascular network is formed prior to implantation into a subject. In some embodiments, one or more other types of cells, e.g., hepatocytes, are co-cultured with endothelial cells, and optionally fibroblasts, to form an organoid or tissue-specific graft, e.g., liver tissue graft. In some embodiments, liver cells e.g. hepatocytes or liver progenitor cells, are co-cultured with endothelial cells, and optionally fibroblasts, to form a pancreatic graft. liver progenitor cells. In some embodiments, pancreatic cells (alpha, beta, gamma, delta) are co-cultured with endothelial cells, and optionally fibroblasts, to form a pancreatic graft. In some embodiments, enterocytes are co-cultured with endothelial cells, and optionally fibroblasts, to form an intestinal graft. In some embodiments, kidney cells, e.g. renal epithelial cells, are co-cultured with endothelial cells, and optionally fibroblasts, to form a kidney graft. In some embodiments, astrocytes, brain cells, neuron, and/or glia cells are co-cultured with endothelial cells, and optionally fibroblasts, to form a neural tissue graft. In some embodiments, muscle cells or myoblasts are co-cultured with endothelial cells, and optionally fibroblasts, to form a muscle graft. In some embodiments, lung or respiratory epithelial cells, e.g., cilia cells, goblet cells, basal cells and/or pneumocytes, are co-cultured with endothelial cells, and optionally fibroblasts, to form a respiratory epithelium graft, e.g., lung, trachea and bronchi graft. In some embodiments, adipocytes are co-cultured with endothelial cells, and optionally fibroblasts, to form an adipose tissue graft. In some embodiments, osteoclasts are co-cultured with endothelial cells, and optionally fibroblasts, to form an bone graft. In some embodiments, stem cells are co-cultured with endothelial cells, and optionally fibroblasts, to form an stem tissue graft.

In some embodiment, endothelial cells, and optionally other cells, e.g., fibroblast and/or hepatocytes, are cultured in the hydrogel for at least 4 days, 5 days, 6 days, or 7 days to a vascular network that is sufficient for implantation.

In some embodiments, the hydrogel is used to generate a scaffold, and endothelial cells are patterned in the hydrogel scaffold to form geometrically defined structures that resemble cylinders, rods, strings, or filaments and networks of such structures. The pre-defined structure provide an architecture for vascular expansion and development in the graft by providing a template for capillary formation. The methods of making and use of an engineered tissue construct wherein endothelial cells are patterned in a defined structure to form a vascular network in a hydrogel scaffold is described in WO 2017/062757.

### Other Embodiments

The disclosure relates to the following embodiments. Throughout this section, the term embodiment is abbreviated as ‘E’ followed by an ordinal. For example, E1 is equivalent to Embodiment 1.
E1. A synthetic heparin mimetic comprising a polymeric carbohydrate backbone of repeating polysaccharide units, each unit having one or more chemically reactive hydroxyl groups, wherein the mimetic is modified at the one or more hydroxyl groups with a functional group to provide a negative charge to the mimetic to promote growth factor binding and/or growth factor activity.
E2. The synthetic heparin mimetic of embodiment 1, wherein the repeating polysaccharide units are the same.
E3. The synthetic heparin mimetic of embodiment 2, which is a homopolymer.
E4. The synthetic heparin mimetic of embodiment 1, wherein the repeating polysaccharide units comprise 2, 3 or more different polysaccharide units.
E5. The synthetic heparin mimetic of any one of embodiments 1-4, wherein the functional group is selected from a sulfate group, a phosphate group, a carboxylic group, and mixtures thereof.
E6. The synthetic heparin mimetic of any one of embodiments 1-5, comprising a zeta potential of −10 to −50 mV.
E7. The synthetic heparin mimetic of any one of embodiments 1-6, having a molecular weight of 70 to 90 kDa.
E8. The synthetic heparin mimetic of any one of embodiments 1-7, wherein each repeating polysaccharide unit comprises 0.5-2.0 functional groups per repeating unit.
E9. The synthetic heparin mimetic of any one of embodiment 1-8, wherein the growth factor is a VEGF, FGF, or combination thereof.
E10. The synthetic heparin mimetic of any one of embodiments 1-9, having reduced anti-coagulant activity relative to heparin.
E11. The synthetic heparin mimetic of any one of embodiments 4-10, wherein the polysaccharide is selected from the group: alginate, agarose, chondroitin sulfate, chitin/chitosan, cellulose, starch, and glycogen.
E12. The synthetic heparin mimetic of any one of embodiments 3 and 5-10, wherein the homopolymer is dextran.
E13. A method for generating the synthetic heparin mimetic of any one of embodiments 1-12, comprising contacting the polymeric carbohydrate backbone with the functional group under conditions that allow for a chemical reaction between the hydroxyl group and the functional group.
E14. A composition comprising a modified dextran molecule having at least one chemically reactive hydroxyl group modified with a sulfate group to provide a negative charge, wherein the modified dextran molecule has a molecular weight of 70-90 kDa and a zeta potential of −20 to −30 mV.
E15. The composition of embodiment 14, wherein the dextran molecule comprises repeating polysaccharide units, each unit comprising 0.5-2.0 sulfate groups per repeating unit.
E16. The composition of embodiment 14 or 15, wherein the negative charge promotes growth factor binding and/or growth factor activity.
E17. The composition of embodiments 16, wherein the growth factor is a VEGF, FGF, or combination thereof.
E18. The composition of any one of embodiments 14-17, having reduced anti-coagulant activity relative to heparin.
E19. A hydrogel comprising a plurality of the synthetic heparin mimetic of any one of embodiments 1-12, wherein the synthetic heparin mimetics are cross-linked via a cross-linker.
E20. A hydrogel comprising a plurality of modified dextran molecules each having at least one chemically reactive hydroxyl group modified with a sulfate group to provide a negative charge, wherein the modified dextran molecules are cross-linked via a cross-linker.
E21. The hydrogel of embodiment 19 or 20, wherein the cross-linker is a cleavable cross-linker.
E22. The hydrogel of embodiment 21, wherein the cleavable cross-linker is a matrix metalloproteinase (MMP)-cleavable dithiol-containing crosslinker peptide.
E23. The hydrogel of embodiment 22, wherein the crosslinker peptide is CGPQGIAGQGCR (SEQ ID NO: 3).
E24. The hydrogel of any one of embodiments 19-23, wherein the synthetic heparin mimetic or the modified dextran molecule is functionalized with methacrylate prior to cross-linking.
E25. The hydrogel of any one of embodiments 19-24, further comprising a cell-adhesive peptide.
E26. The hydrogel of embodiment 20, wherein the cell-adhesive peptide is CGRGDS (SEQ ID NO: 1).
E27. The hydrogel of any one of embodiments 18-26, further comprising at least one growth factor.
E28. The hydrogel of embodiment 21, wherein the at least one growth factor is a VEGF, FGF, or combination thereof.
E29. The hydrogel of any one of embodiments 19-28, further comprising at least one population of cells.
E30. The hydrogel of any one of embodiments 19-28, further comprising at least two different populations of cells.
E31. The hydrogel of embodiment 29 or 30, wherein the cells are capable of forming multicellular sprouts, and wherein the number of multicellular sprouts is increased relative to a hydrogel without the heparin mimetic or modified dextran molecule.
E32. A method of increasing vascularization of an engineered tissue construct in a subject, comprising administering to the subject the engineered tissue construct in combination with the synthetic heparin mimetic of any one of embodiments 1-12, the composition of any one of embodiments 14-18, or the hydrogel of any one of embodiments 19-31, wherein vascularization is increased relative to an engineered tissue construct administered without the heparin mimetic, the composition, or the hydrogel.
E33. A method of increasing survival of an engineered tissue construct in a subject, comprising administering to the subject the engineered tissue construct in combination with the synthetic heparin mimetic of any one of embodiments 1-12, the composition of any one of embodiments 14-18, or the hydrogel of any one of embodiments 19-31, wherein survival is increased relative to an engineered tissue construct administered without the heparin mimetic, the composition, or the hydrogel.
E34. A method of increasing engraftment of an engineered tissue construct in a subject, comprising administering to the subject the engineered tissue construct in combination with the synthetic heparin mimetic of any one of embodiments 1-12, the composition of any one of embodiments 14-18, or the hydrogel of any one of embodiments 19-31, wherein engraftment is increased relative to an engineered tissue construct administered without the heparin mimetic, the composition, or the hydrogel.
E35. A method of promoting angiogenesis in a diseased tissue in a subject, comprising administering to the subject the synthetic heparin mimetic of any one of embodiments 1-12, the composition of any one of embodiments 14-18, or the hydrogel of any one of embodiments 19-31.
E36. The method of embodiment 35, wherein the diseased tissue comprises a region of ischemia.
E37. A hydrogel comprising a plurality of modified dextran molecules conjugated with heparin, wherein each modified dextran molecule comprises repeating units comprising at least one chemically reactive hydroxyl group modified with a sulfate group to provide a negative charged, and wherein the dextran molecules are cross-linked via a crosslinker.

### INCORPORATION BY REFERENCE

All documents and references, including patent documents and websites, described herein are individually incorporated by reference to into this document to the same extent as if there were written in this document in full or in part.

### EQUIVALENTS

Those skilled in the art will recognize or be able to ascertain, using no more than routine experimentation, many equivalents of the specific embodiments described herein. Such equivalents are intended to be encompassed by the following claims.

### EXAMPLES

### Example 1: Generation of Heparin-Dextran Hydrogels

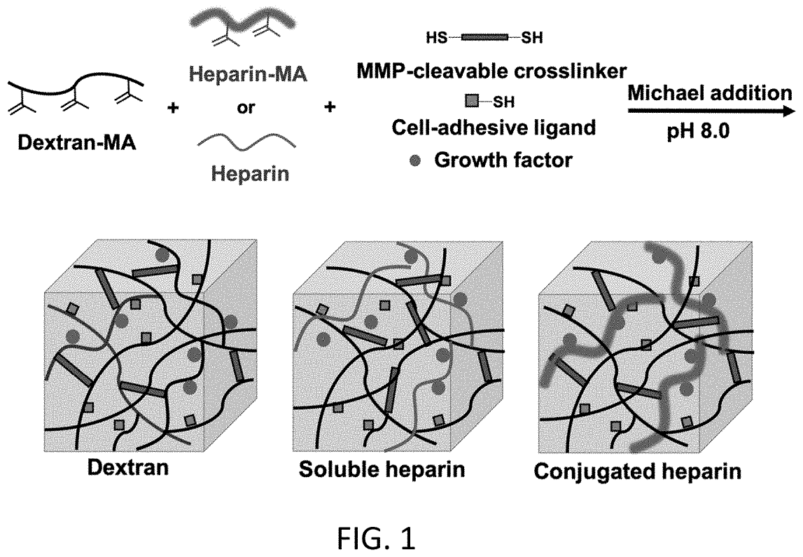

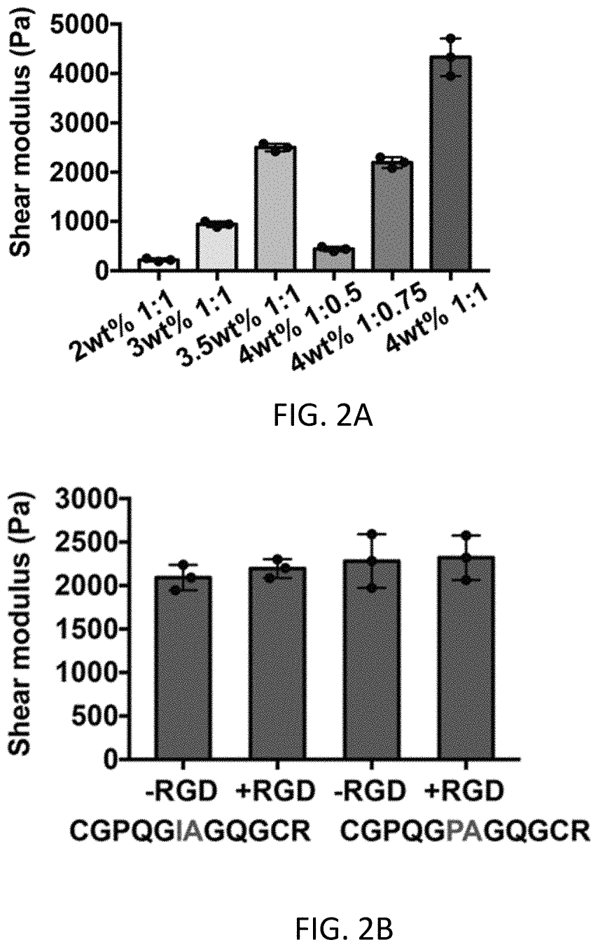

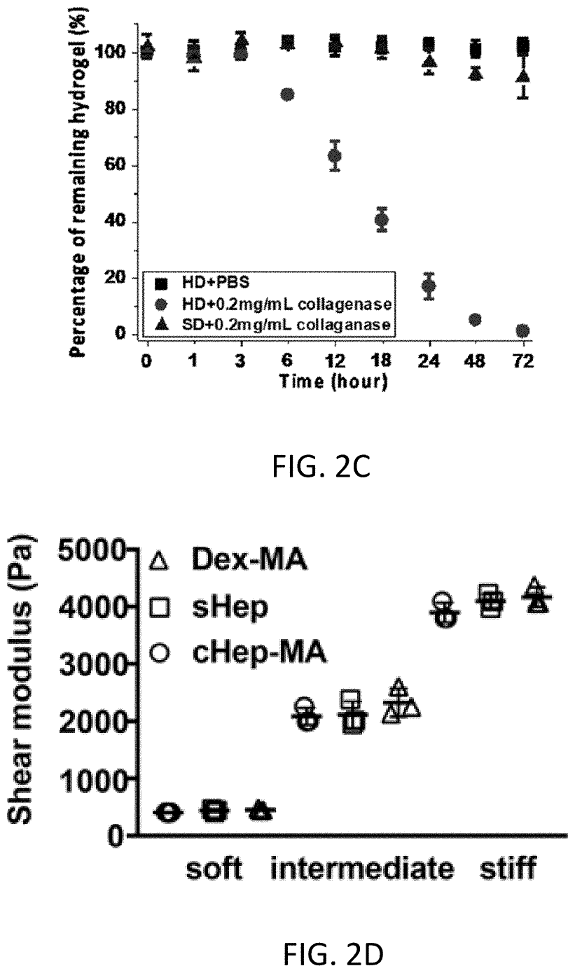

To generate a new material that mimics the pro-angiogenic activity of heparin-conjugated biomaterials but eliminates its shortcomings, a synthetic material system using dextran, a widely used polysaccharide in clinical settings, was generated. Dextran is intrinsically biocompatible and bio-inert with no known cell surface receptor binding activity, and has structural features similar to the glycosylated layer of native extracellular matrices. Cell interactive, biomimetic hydrogels were formed by reacting methacrylate functionalized dextran macromers with di-thiolated metalloproteinase (MMP)-cleavable crosslinkers, and thiol-terminated RGD peptides, through Michael-type addition reaction (FIG. 1). As described previously, a combination of integrin-binding sites and substrates for MMPs renders the matrix degradable and invasive by cells that secrete MMPs (see e.g., Lutolf, et al. PNAS (2003) 100:5413-5418). In this synthetic system, multiple material properties were modulated, such as hydrogel stiffness (through adjusting bulk material solution concentrations crosslinking density (FIG. 2A)), cell adhesiveness (through coupling different concentrations of cell-adhesive RGI) peptide (FIG. 2B)) and matrix degradation (through varying the MMP-labile crosslinker sequences (FIG. 2C). To support enhanced angiogenesis, heparin was chemically conjugated to the dextran gels (FIG. 1). A soluble non-modified heparin was used as a control during hydrogel crosslinking. The incorporation of conjugated heparin or soluble heparin into the dextran hydrogel did not impact the ability to control materials parameters (FIG. 2D).

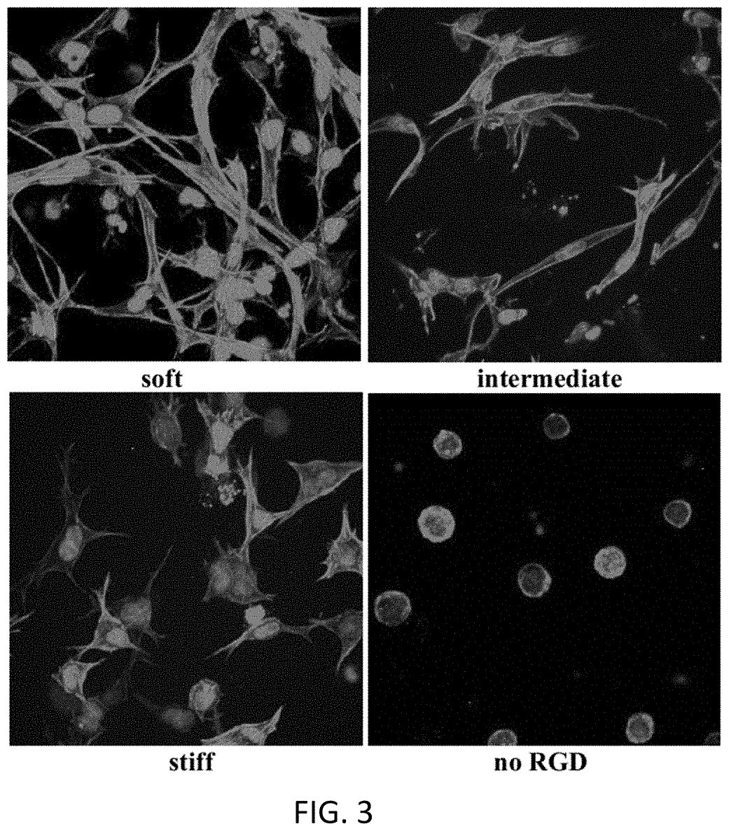

Further assessment analyzed cell-adhesiveness of HDFs in the hydrogel (FIG. 3). Specifically, the effect of hydrogel composition on morphology of encapsulated human dermal fibroblasts (HDFs) was evaluated. Dextran hydrogels with variable stiffness (modulated by altering the crosslinking density) or containing no RGD peptide were prepared and seeded with GFP-expressing HDFs at 1×10⁶/mL. Images of the hydrogels were taken by confocal microscopy at 3 days following cell encapsulation. As shown in FIG. 3, soft hydrogels favored adhesive morphology of the HDFs, while stiff hydrogels or hydrogels lacking RGD contained HDFs lacking focal adhesions exhibiting minimal cell spreading.

### Example 2: Incorporation of Heparin in Hydrogel Enhances Vascular Network Formation In Vitro

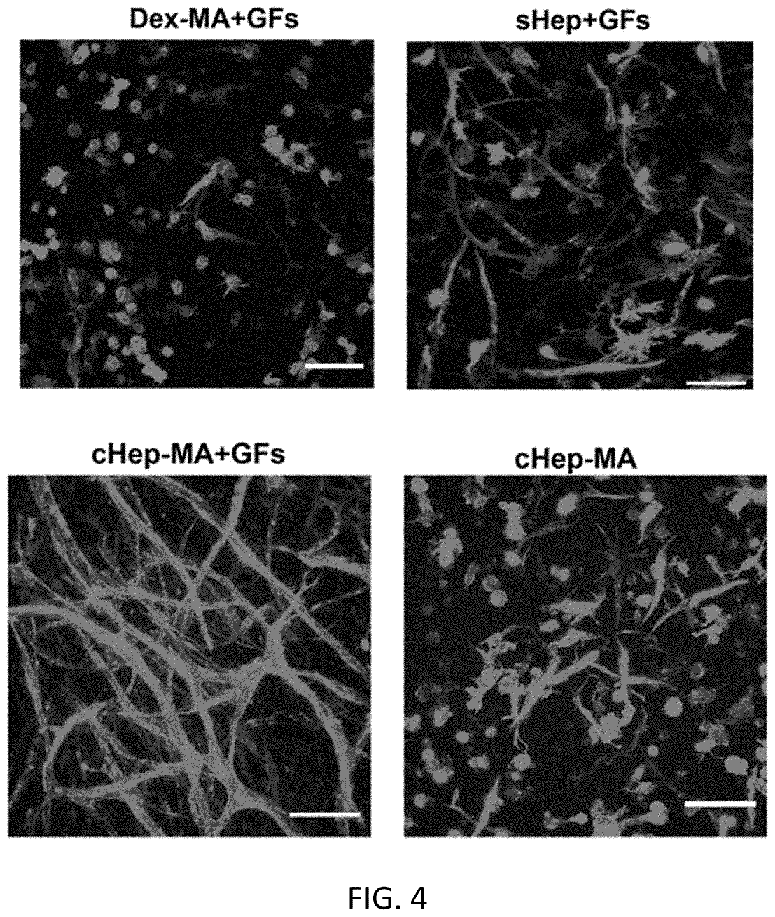

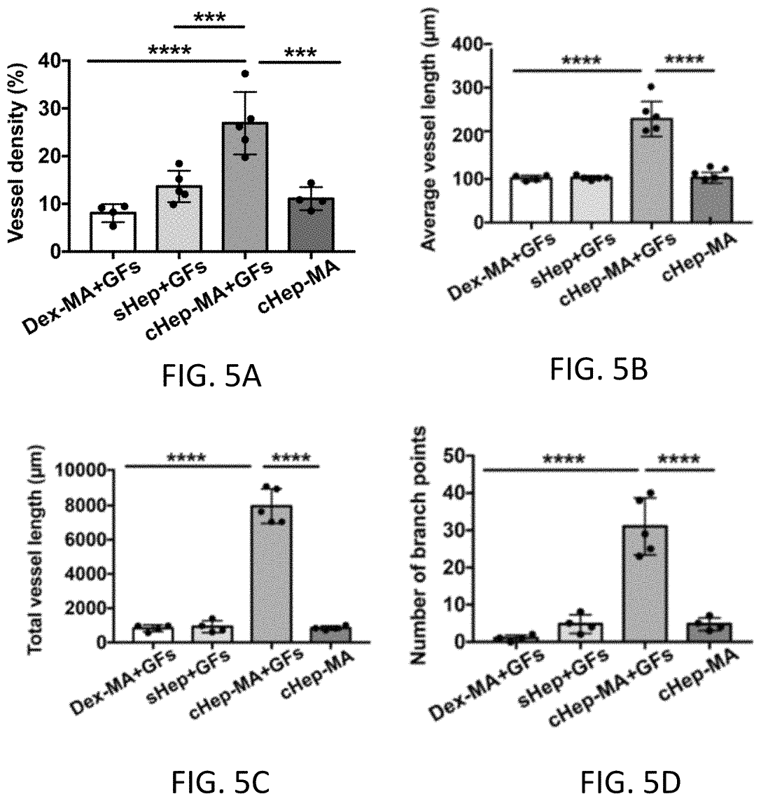

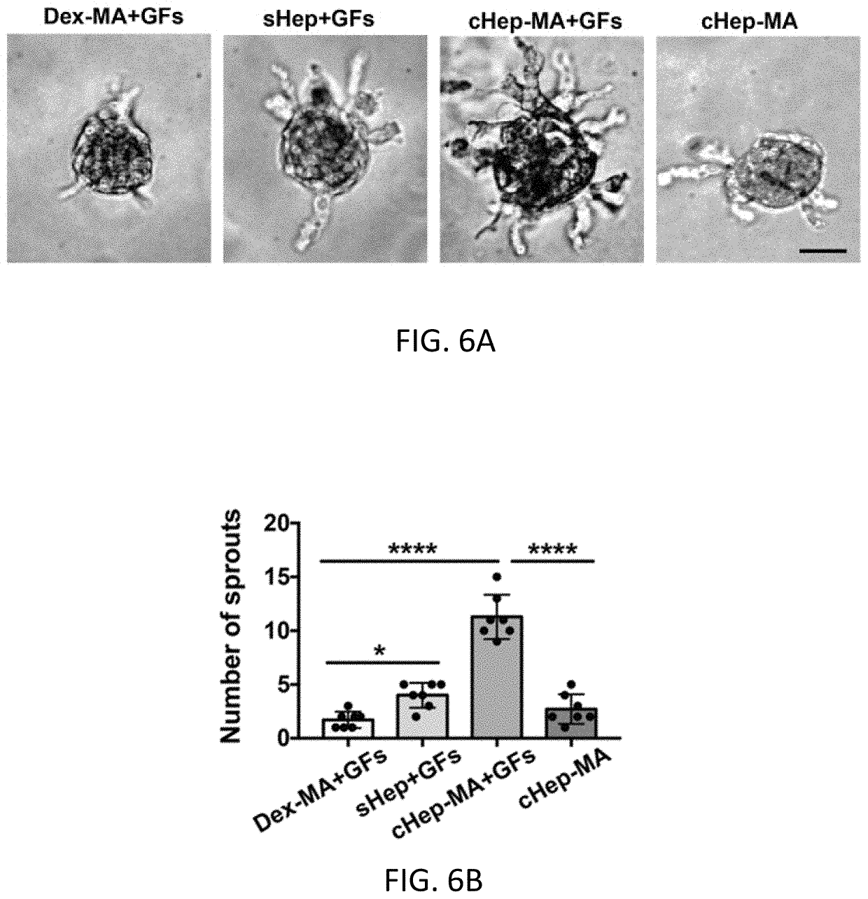

Having established a synthetic material platform, the effect of hydrogel composition and heparin incorporation on vascular network formation in culture was examined. To assess vasculogenesis, human umbilical vein endothelial cells (HUVECs) expressing Ruby-LifeAct and human dermal fibroblasts (HDFs) expressing GFP were encapsulated and co-cultured in various dextran-based hydrogels. After 14 days, endothelial cells assembled robust multicellular networks featuring higher densities of longer vessels with numerous branch points and defined lumen structures only in dextran gels with conjugated heparin and impregnated vascular endothelial growth factor (VEGF) and basic fibroblast growth factor (bFGF) (cHep-MA+GFs), while no vascular structures were apparent in hydrogels with no heparin (Dex-MA+GFs), soluble heparin (sHep+GFs) or conjugated heparin without added growth factors (cHep-MA) (FIGS. 4 and 5A-5D). These findings were recapitulated using an angiogenic sprouting assay, wherein encapsulated HUVEC. spheroids exhibited different extents of sprouting and invasion of endothelial cells depending on hydrogel compositions; cHep-MA+GFs induced significantly more multicellular sprouts compared to all other gel formulations (FIGS. 6A-6B).

### Example 3: Incorporation of Heparin in Hydrogel Enhances Vascular Network Formation In Vivo

To test whether enhanced vascular network formation would translate in vivo, the hydrogel compositions of Example 2 were introduced subcutaneously in mice and vascularization was assessed.

Specifically, heparinized dextran hydrogels were compared to no heparin (Dex-MA+GFs), soluble heparin (sHep+GFs), or no growth factors (cHep-MA) conditions, i.e., vs. heparin-conjugated dextran with growth factors (cHep-MA+GFs), at day 14 following injection in mice. In particular, heparin-conjugated dextran hydrogels containing VEGF and bFGF growth factors (cHep-MA+GFs), dextran hydrogels containing no heparin and VEGF and bFGF growth factors (Dex-MA+GFs), dextran hydrogels containing soluble heparin and VEGF and bFGF growth factors (sHep+GFs), or heparin-conjugated dextran hydrogels containing no growth factors (cHep-MA) were examined. The hydrogels were subcutaneously injected as well as preformed and implanted into mice and evaluated for vascularization at 14 days following injection.

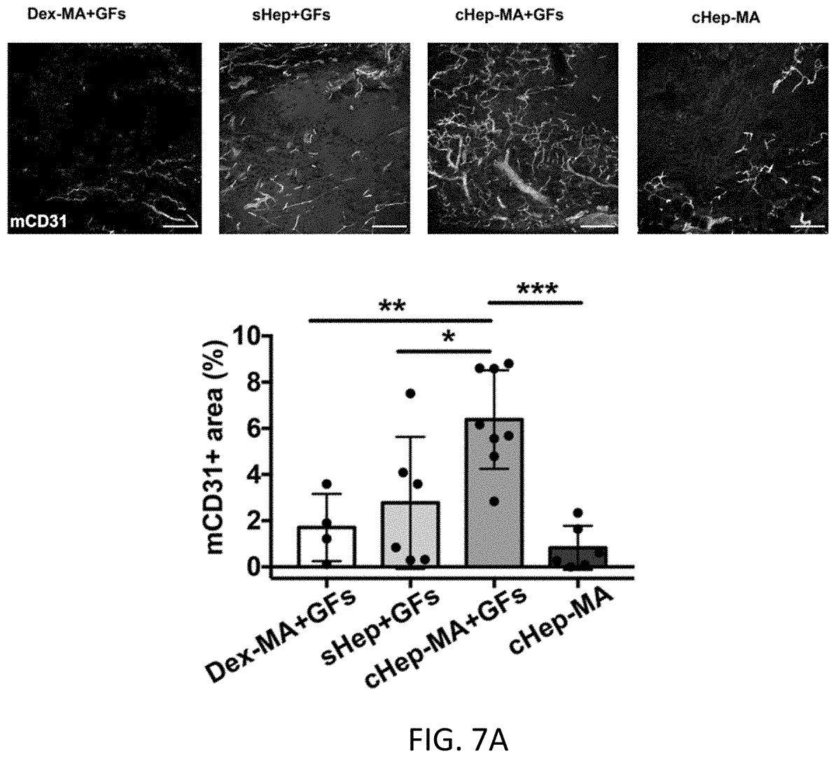

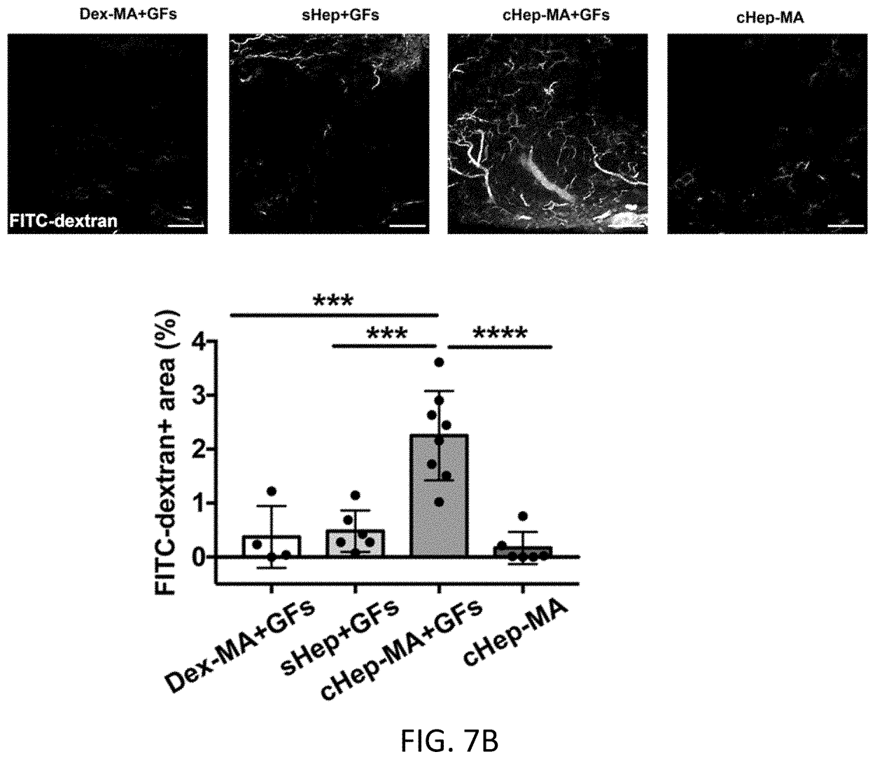

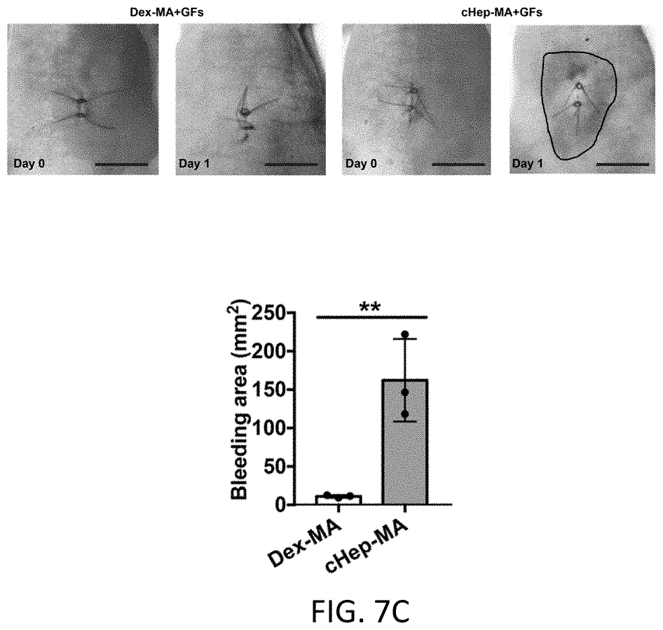

The results are shown in FIGS. 7A-7B. Hydrogels induced significant invasion of host vasculature and exhibited the highest degree of vascular invasion as measured by the presence of CD31+ host endothelial cells (FIG. 7A), i.e., with the heparin-conjugated dextran hydrogels (cHep-MA+GFs) inducing the highest degree of vascular invasion as measured by CD31+ host endothelial cells in hydrogels harvested from the mice heparin. Moreover, these invading vessels featured hierarchical branching networks and established connectivity to the systemic circulation as demonstrated by perfusion of 70 kDa FITC-dextran that was injected intravenously prior to tissue harvesting (FIG. 7B).

While these initial studies confirmed that heparin conjugation in biomaterials is an effective strategy to enhance in vivo vascularization, it was observed that animals receiving heparin-containing hydrogels experienced persistent local bleeding during implantation and post-operatively, significant bruising around the implantation sites (FIG. 7C), and in 30-50% of the cases resulted in impaired mobility and morbidity due to prolonged bleeding from the wounds. These observations highlight safety concerns of incorporating heparin for translational applications, and prompted investigation in to whether a synthetic heparin mimetic could be developed that recapitulates the observed pro-angiogenic benefits without the anticoagulant drawbacks of native heparin.

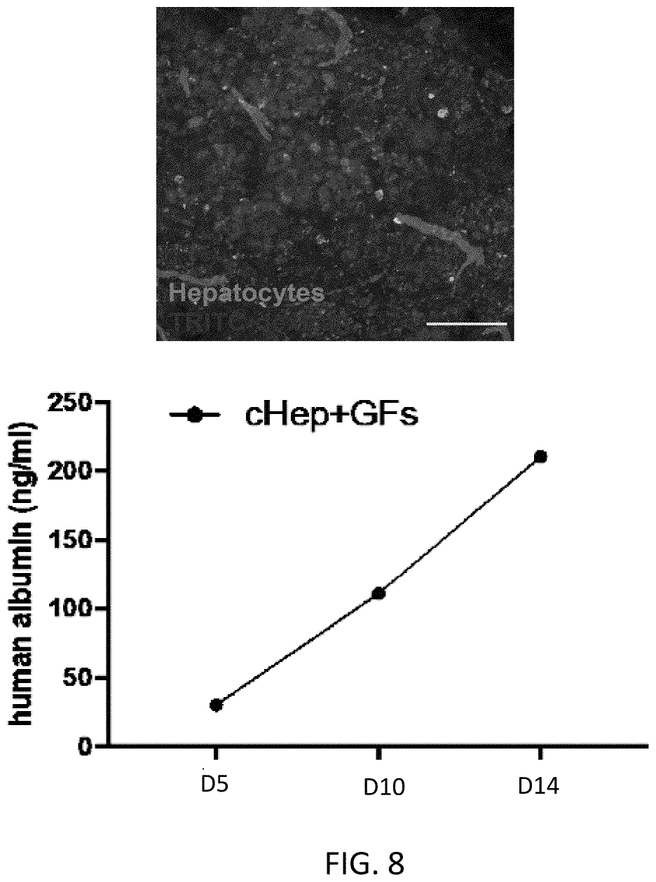

Additionally, to test whether the engineered pro-angiogenic biomaterials can be used for tissue engineering applications, we further examine if the vascular network formation formed in the composition of Example 2 is sufficient to support survival and function of other cells in vivo, human hepatocyte aggregates containing dermal fibroblasts at 1:2 ratio co-encapsulated in dextran-based hydrogels with different heparin content in the presence of VEGF and bFGF (50 ng/mL) were generated. Hydrogels containing the hepatocyte aggregates were injected in mice and were found to enhanced in vivo host vessel invasion and tissue engraftment (FIG. 8, top). Blood was collected at days 5, 10, 14 for albumin measurements (FIG. 8, bottom) and showed tissue function with albumin production. Specifically, the concentration of human albumin in serum was measured, and was found to increase over time following implantation of the hydrogels. These results indicate that the host mouse vessels invaded into the heparin conjugated material. These results further indicate that the implanted human hepatocytes were effectively functioning in vivo to produce albumin.

### Example 4: Engineering Sulfated Dextran Polymers that Mimic the Pro-Angiogenic Properties of Heparin

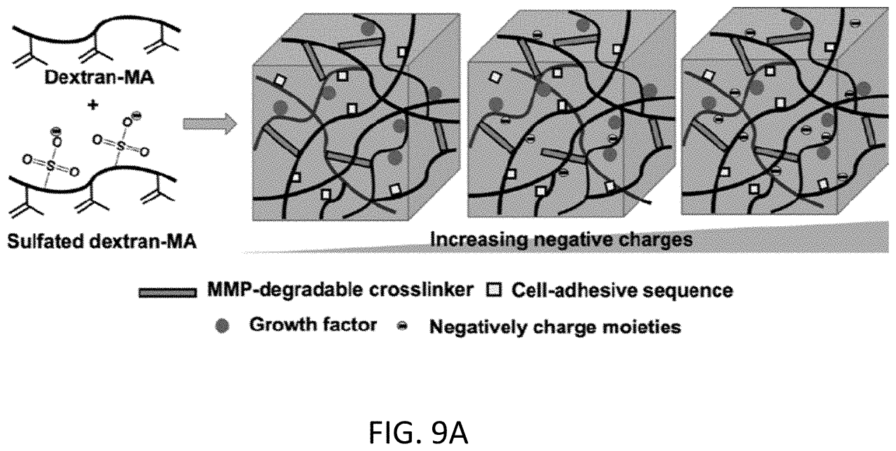

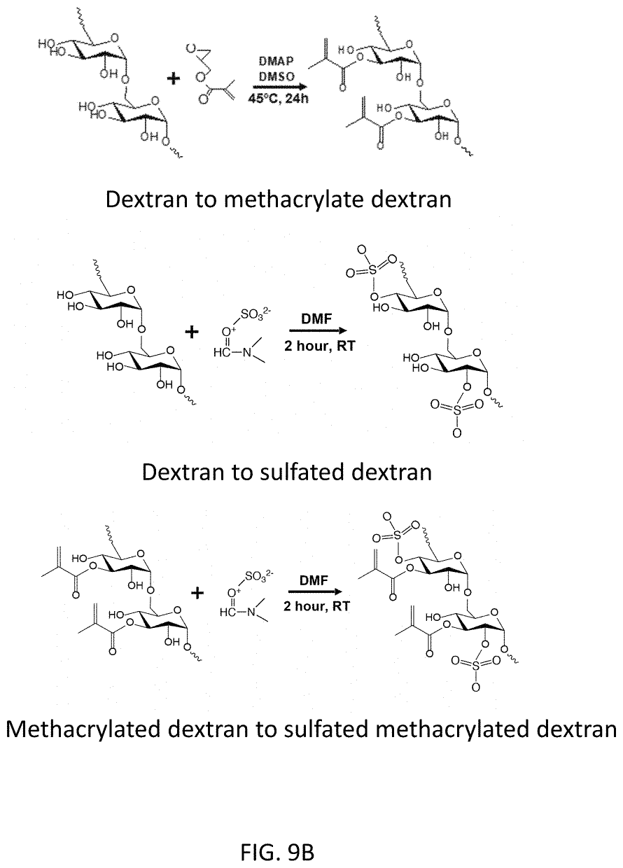

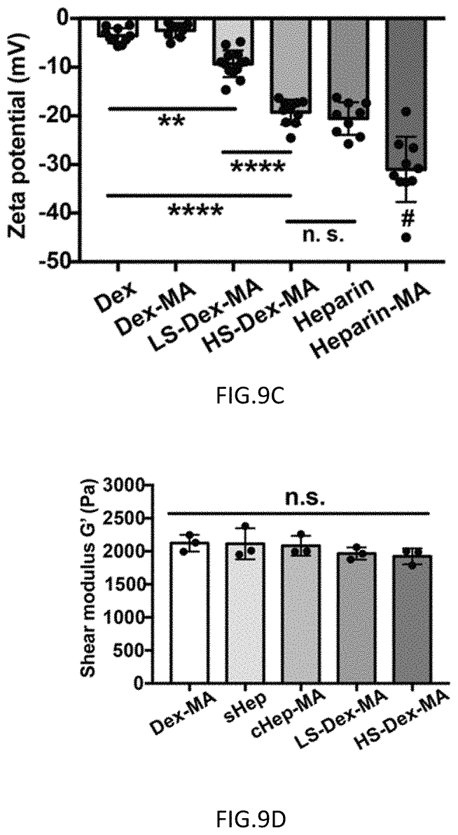

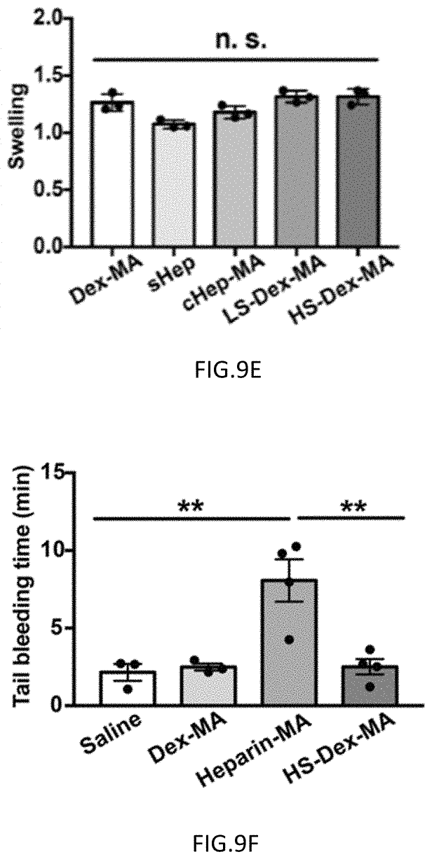

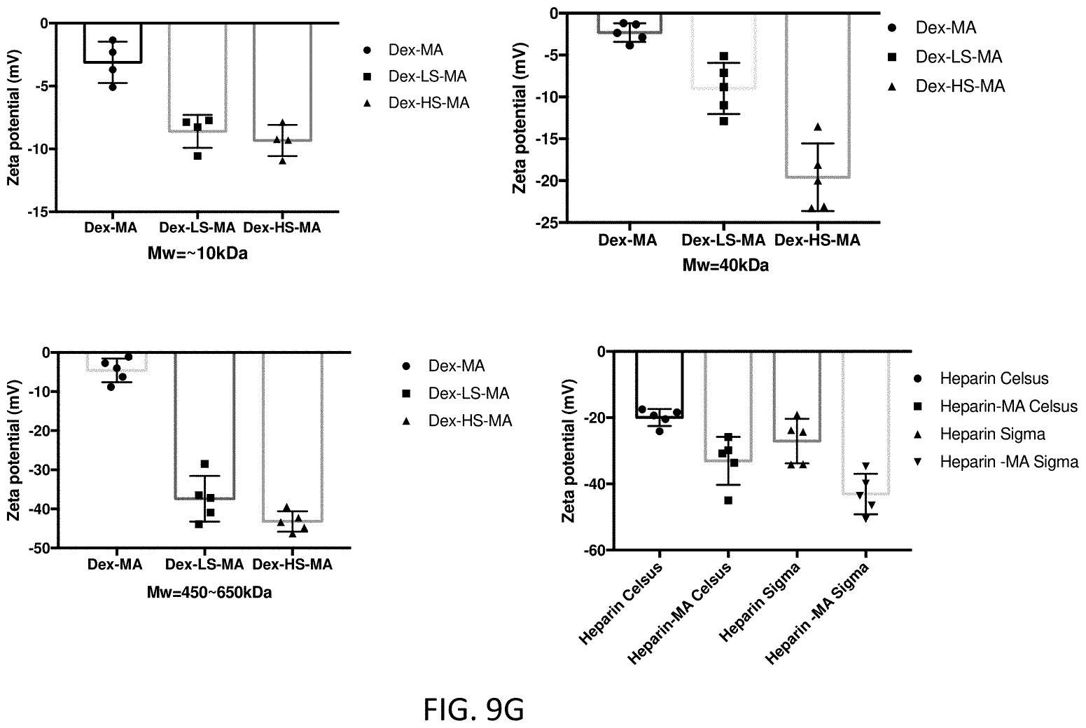

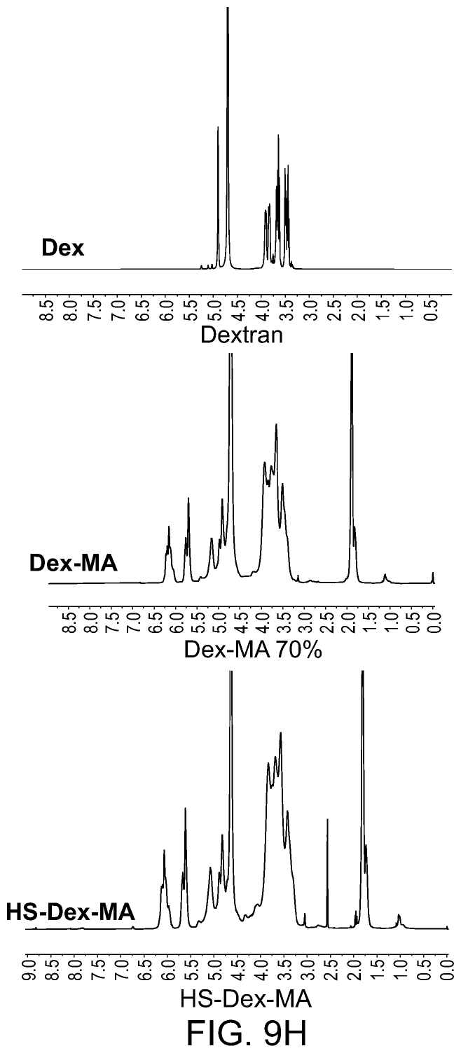

The pro-angiogenic property of heparin arises from its affinity to bind growth factors, which is mediated through electrostatic interactions of the high density of negatively charged sulfate groups of heparin. To assess the effects of charge and sulfation on dextran, charge adducts were introduced to the dextran backbone through sulfation. A sulfur trioxide/DMF complex was used to sulfate the dextran (FIGS. 9A-9B). The degree of sulfation was altered resulting in a highly sulfated dextran (HS-Dex-MA), and a lower sulfated dextran (LS-Dex-MA). As shown in FIG. 9B, the sulfation reaction was used to sulfate unmodified dextran (mean weight-average MW was approximately 86 kDa with a range of 60-90 kDa) or dextran first modified with methacrylate (Dex-MA). The degree of sulfation was altered in order to provide a highly sulfated methacrylated dextran (HS-Dex-MA), and a lower sulfated methacrylated dextran (LS-Dex-MA). The degree of sulfation was measured by titration or element analysis. The highly sulfated dextran contained >1 sulfates per monomer unit or >20% sulfur content and the low sulfated dextran contained <0.6 sulfates per monomer unit or <9% sulfur content.

The HS-Dex-MA had a degree of sulfation that was comparable to native heparin and heparin-conjugated dextran, as confirmed by a dimethylmethylene blue (DMMB) colorimetric assay (data not shown) and changes in zeta potential of the backbone (FIG. 9C). Additionally, NMR spectra of Dex, Dex-MA and HS-Dex-MA shows that sulfation modification did not change methacrylation degree (FIG. 9H).

The HS-Dex-MA and LS-Dex-MA polymers were crosslinked using a MMP-degradable crosslinker (SEQ ID NO: 3) to prepare hydrogels as described in Example 1. The hydrogels were formed by crosslinking methacrylated dextran (Dex-M A) and sulfated Dex-MA at a ratio of 80:20 (w/w (%)). The chemical modifications did not appear to alter mechanical properties of hydrogels generated from these sulfated dextrans compared to dextran hydrogel (Dex-MA), dextran hydrogel containing soluble heparin (sHep), or heparin-conjugated dextran hydrogels (cHep-MA), as demonstrated by oscillatory shear rheology and swelling (FIGS. 9D-9E). Together, these data suggest that scaffold sulfation can be modified independent of matrix stiffness and swelling.

Zeta potentials were further measured on sulfated dextran of increasing mean weight-average molecular weights, including ˜10, ˜40 and ˜500 kDa. These results show that more negative zeta potentials were achieved in dextran with higher molecular weight (FIG. 9G).

### Example 5: Dextran Sulfation does not Impair Blood Coagulation

To test the effects of the HS-Dex-MA on coagulation as compared to heparin, a mouse tail bleeding assay following systematic infusion through subcutaneously implanted osmotic mini-pumps was performed. Briefly, unconjugated/unsulfated dextran hydrogel (Dex-MA), heparin-conjugated dextran hydrogel (cHep-MA), or HS-Dex-MA hydrogel was loaded in osmotic mini-pumps and implanted under the dorsal skin of mice. At 36 hours following implantation, the mice were euthanized and the tail bleeding time was evaluated. Implantation of the heparin-conjugated dextran hydrogel impaired blood coagulation and elicited a much longer clotting time (˜8 mins) compared to saline (˜2.2 mins) and unmodified dextran (˜2.5 mins) conditions (FIG. 9F). In contrast, the synthetic heparin mimetic (sulfated dextran) did not alter clotting time (˜2.5 mins).

### Example 6: Sulfated Dextran Enhances Growth Factor Signaling

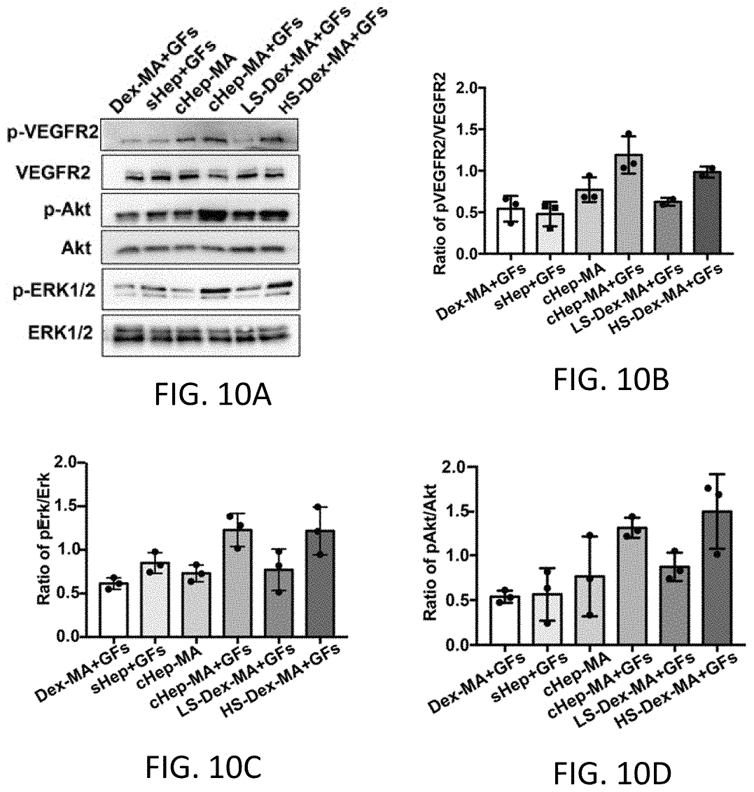

The key bioactivity of interest in heparin is its ability to enhance growth factor signaling. In a VEGF-R2 knock-out model of HUVECS, cells failed to form vascular networks in heparinized hydrogels in the presence of VEGF and bFGF (data not shown), demonstrating the importance of the growth factor signaling. To test whether the sulfated dextrans when incorporated into hydrogels can enhance growth factor signaling, endothelial cells were cultured on various growth factor-laden hydrogels. Specifically, highly sulfated dextran hydrogel (HS-Dex-MA) was prepared with VEGF and bFGF loaded. Comparison was made to low sulfated dextran hydrogel (LS-Dex-MA), heparin conjugated dextran hydrogel (cHep-MA), or dextran hydrogel (Dex-MA) loaded with VEGF and bGFG. Heparin-conjugated dextran hydrogel (cHep-MA) without growth factors and soluble heparin (sHep) combined with VEGF and bFGF were used as controls. The activation of VEGFR2 and ERK1/2 and Akt, two key signaling pathways downstream of VEGF receptor signaling were examined in sulfated dextran by western blot. Cells cultured on growth-factor containing dextran hydrogels formulated with conjugated heparin (cHep-MA GFs) or highly sulfated dextran (HS-Dex-MA+GFs) showed elevated and comparable levels of VEGFR2, Erk1/2 and Akt phosphorylation via western blot analysis (FIGS. 10A-10D), as compared to all other hydrogel compositions, suggesting that sulfated dextran retains the growth factor-enhancing activity of native heparin, but without the anticoagulant activity.

### Example 7: Sulfated Dextran Hydrogels Support Vascularization In Vitro

Having determined that sulfated dextran could be a potential heparin mimetic that preserves growth factor signaling effects without anti-coagulant bioactivity, it was next evaluated whether sulfated dextran hydrogels support vascularization using various in vitro assays.

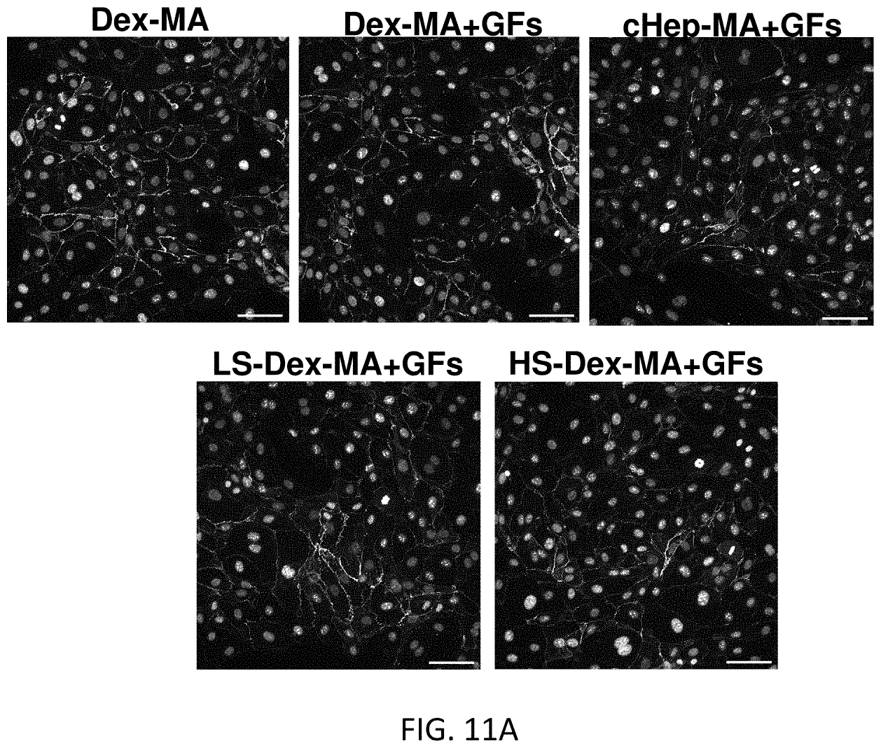

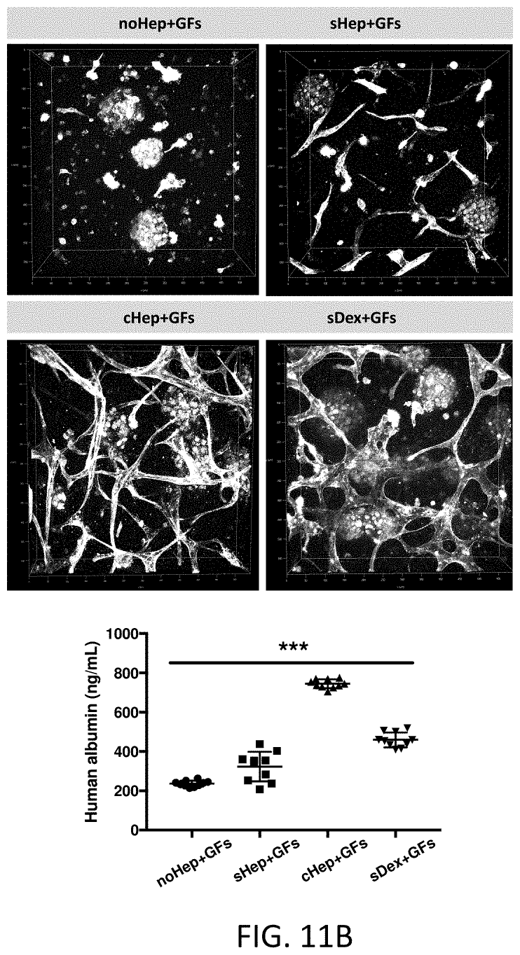

Comparison was made of highly sulfated dextran hydrogels and low sulfated dextran hydrogels, each prepared with VEGF and bFGF growth factors. Specifically, different dextran hydrogels were evaluated for their ability to support cell survival and proliferation using HUVECS. The results showed that the different dextran hydrogels supported cell survival, cell attachment and proliferation of HUVECs (FIG. 11A).

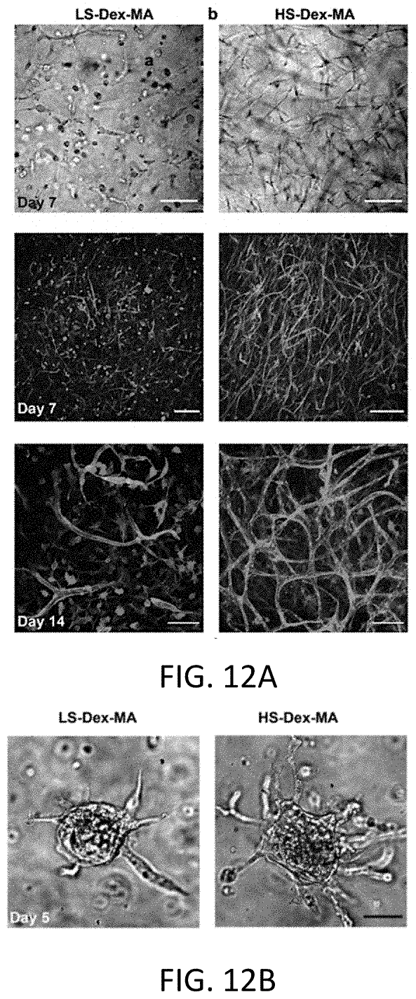

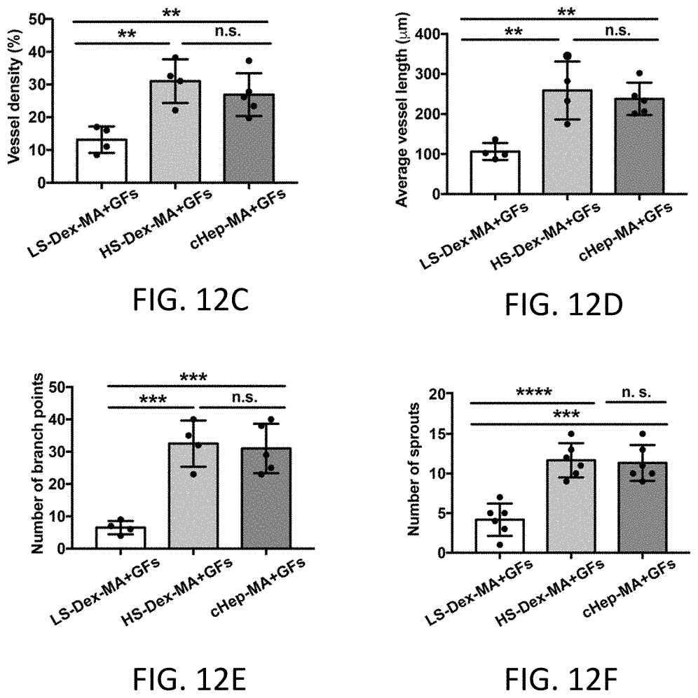

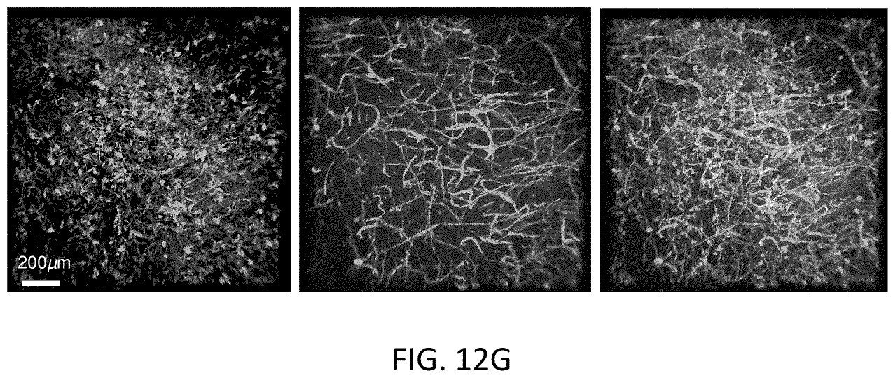

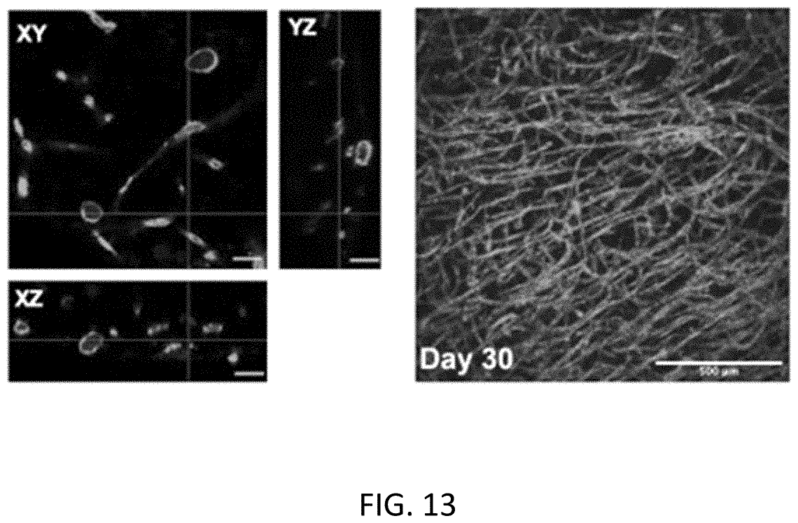

Next, in vitro vascular network formation was examined using previously established sprouting and vasculogenesis assays, through co-culturing of Ruby-Lifeact-HUVECs and GFP-HDFs in sulfated dextran hydrogels. HS-Dex-MA demonstrated substantial sprouting and the formation of extensive vascular networks that were comparable to those observed in the heparin-conjugated composition (see e.g., FIG. 5B), and significantly higher than those observed for all other control conditions, including LS-Dex-MA (FIGS. 12A-12B). Quantitative analysis in HUVEC-aggregates confirmed comparable values in vessel density, vessel length, number of branch points, and number of sprouts between highly sulfated dextran hydrogels loaded with VEGF and bFGF growth factors (HS-Dex-MA+GFs) and heparin-conjugated dextran hydrogel loaded with growth factors (cHep-MA-GFs) conditions (FIG. 12C-12F). Interconnected lumens could be observed in the self-assembled microvasculature formed in HS-Dex-MA GFs hydrogels (FIG. 13 left), and persisted and maintained vascular integrity over a month in culture (FIG. 13 right).

In additional experiments, the ability of hydrogels comprising high molecular weight sulfated dextran to support in vitro vascularization was examined. The results show that hydrogel comprising high molecular weight sulfated dextran in the 400-600 kDa range (˜550 kDa in average) with low zeta potential (˜47 mV) were also able to support in vitro vascularization (FIG. 12G).

Finally, to determine if the sulfate dextran hydrogel is suitable for promoting vascularization in tissue constructs for tissue engineering applications, human hepatocyte aggregates containing dermal fibroblasts were co-encapsulated with Ruby-HUVECs in the presence of VEGF and bFGF in sulfated dextran-based hydrogels (sDex+GFs), dextran-based hydrogel loaded with soluble heparin and VEGF and bFGF (sHep+GFs), dextran-based hydrogel loaded with VEGF and bFGF only (noHep+GFs) and heparin-conjugated dextran hydrogel loaded with VEGF and bFGF (cHEN+GFs). Vascularization was observed in hydrogels having human hepatocyte aggregates, and the greatest vascularization was observed with the sulfated dextran hydrogels (FIG. 11B, top images). Albumin production by the hepatocytes in sulfated dextran-based hydrogels was also better than dextran-based hydrogels with soluble heparin (sHep+GFs) (FIG. 11B, bottom graph). The heparin-mimetic sulfated dextran hydrogels were able to support the vascularization of human hepatocytes and promoted organ level of function.

### Example 8: Sulfated Dextran Supports Angiogenesis In Vivo

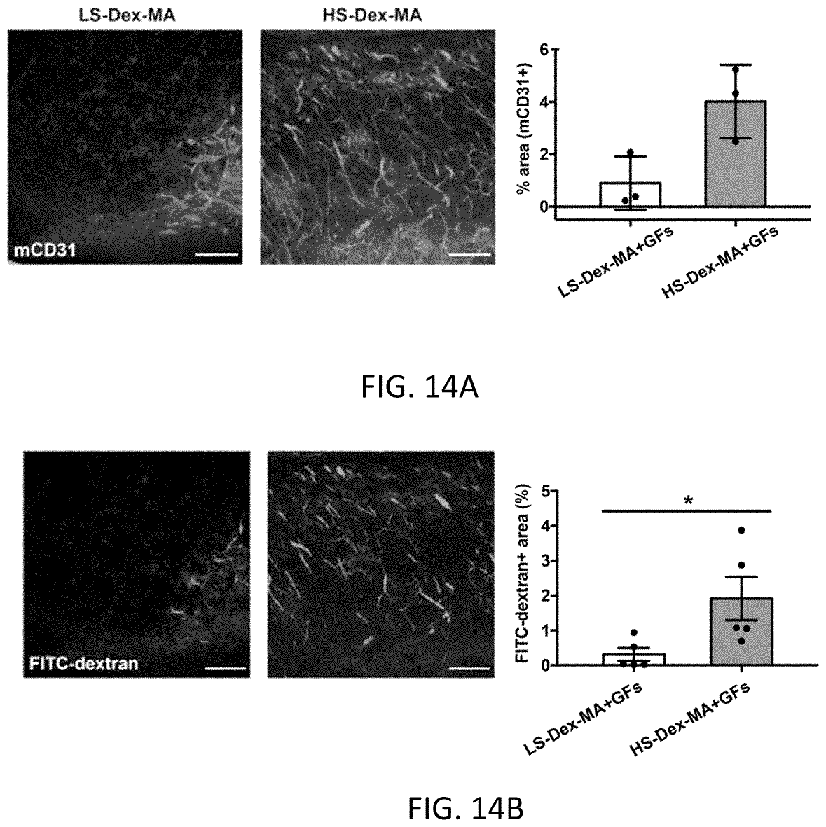

To investigate whether the sulfated dextran-based material could support angiogenesis in vivo the mouse model used in Example 3 was injected with the sulfated dextran hydrogels described in Example 4. Dextran gels featuring a high degree of sulfation induced substantial angiogenesis, as evidenced by substantial endothelial network invasion in tissue sections, measured by mCD31 staining (endothelial cell marker) (FIG. 14A) and perfusion visible by intravenously injected FITC-dextran (FIG. 14B), while low sulfation compositions did not. Importantly, no evidence of microvascular bleeding, bruising, or loss of mobility in animals as a result of exposure to the sulfated dextran hydrogels upon implantation was observed. Together, these data demonstrate a generated synthetic heparin mimetic that lacks anticoagulation activity without compromising its activity to promote tissue vascularization.

### Materials and Methods

Chemical synthesis of methacrylated dextran. Dextran (Mw˜86 kDa, MP Biomedicals) was functionalized with methacrylate groups according to a previous published protocol (van Dijk-Wolthuis, W. N. E. et al. Macromolecules 28, 6317-6322 (1995)) Briefly, dextran (2.0 g) was dissolved in 10 mL anhydrous dimethyl sulfoxide (DMSO) with the addition of 0.2 g of base catalyst 4-dimethylamino pyridine (DMAP) and desired molar equivalent of glycidyl methacrylate (GMA, density=1.042 g/mL at 25° C.). The solution mixture was kept constant at 45° C. and stirred for 24 hours before precipitating the final product via pipetting in drop-by-drop fashion of dark brown reaction solution to 100 mL ice-chilled isopropanol. The crude product was then collected via centrifugation, re-dissolved in milli-Q water and dialyzed against milli-Q water (at 4° C.) for 3 days with 3 changes (4 L) daily before lyophilization. The degree of dextran methacrylate functionality was characterized via ¹H NMR spectroscopy, confirming a 70% modification (70 conjugated methacrylate groups per 100 dextran glucopyranose residues).

Chemical synthesis of methacrylated heparin. Heparin (sodium salt from porcine intestinal mucosa, Mw˜16 kDa, Sigma) was modified with methacrylate groups following a previously published method. Briefly, 5% w/v heparin in milli-Q H₂O was prepared and reacted with 5-fold molar excess of methacrylic anhydride. The pH of the reaction mixture was adjusted to 8.5 using 5 N NaOH, and the reaction was proceeded overnight at 4° C. The product was then precipitated in 95% ethanol, dried and dialyzed (3000 Mw cutoff) for 3 days in milli-Q H₂O and lyophilized. The degree of methacrylation was characterized via ¹H NMR spectroscopy, confirming an average of 16% methacrylation.

Chemical synthesis of sulfated dextran. Methacrylate modified dextran was modified following a previous published method. Briefly, Dex-MA (0.5 wt %) was dissolved in N, N-dimethylformadmide (DMF) with various amount of SO₃/DMF complex added to the reaction solution to achieve a range of molar ratio of SO3/DMF:Dex-MA repeat unit (e.g., 1:1, 5:1 and 10:1, mol/mol), a means to tune the degree of sulfation in final product. The solution mixture was reacted under N₂ at room temperature for 1 hour followed by dialysis (10000 Mw cutoff) against milli-Q H₂O at 4° C. for 7 days and lyophilized.

Dextran-based hydrogel formulation. 3D dextran-based hydrogels were prepared via mixing Dex-MA (100 mg/mL) with 8 mM thiolated RGD peptide (cell-adhesive sequence: CGRGDS (SEQ ID NO: 1); non-adhesive control: CGRGES (SEQ ID NO: 2), Aapptec) in the presence of matrix metalloproteinase (MMP)-cleavable dithiol-containing crosslinker peptide (degradable crosslinker: CGPQGIAGQGCR (SEQ ID NO: 3), derived from collagen I; slow-degradable control: CGPQGPAGQGCR (SEQ ID NO: 4), Aapptec) in M199 media containing sodium bicarbonate (3.5% w/v) and HEPES (10 mM). To formulate heparinized dextran hydrogels, Dex-MA precursor solution was mixed with either heparin-MA (100 mg/mL) or non-modified heparin (100 mg/mL) at 90:10 w/w ratio. To formulate sulfated dextran hydrogels, Dex-MA precursor solution was mixed with sulfated Dex-MA (100 mg/mL, at low and high sulfation degree) at 80:20 w/w ratio. The pH of the solution was then adjusted approximately to 8 with NaOH (1 M) to initiate hydrogel formation through Michael-type addition reaction and maintained for 45 minutes at 37° C. for complete gelation. To formulate hydrogels with different stiffness, bulk material solution concentration or MMP-labile peptide crosslinker density can be tuned independent of other material parameters during crosslinking.

Mechanical characterization via oscillatory rheology. Bulk hydrogel mechanical properties were measured using a strain-controlled Discovery HR-2 oscillatory shear rheometer (TA Instruments, New Castle, Del.), with a 20-mm diameter cone-on plate geometry, 2° cone angle and at a 62 μm gap distance at 37° C. Hydrogels with various compositions were prepared as described above. Hydrogel precursor solutions were deposited onto the rheometer Peltier plate for in situ mechanical stiffness measurements. To determine hydrogel formation and gelation kinetics, a time sweep was first performed at a constant 6 rad/s frequency, 1% strain; followed by a frequency sweep conducted over a logarithmic scale from 0.1 rad/s to 100 rad/s at a fixed strain amplitude of 1% to confirm the mechanical stability of resulting hydrogels. Data were collected from multiple measurements of 4 independent samples.

Hydrogel swelling. Dextran hydrogels (˜200 μL, 4 wt % polymer concentration) with various compositions were prepared as described above and their in situ weights after crosslinking were measured. Samples were then immersed in PBS and hydrogel swollen weights were measured after 24 hours incubation at 37° C. The swelling degree of hydrogels is calculated by dividing swollen weight over in situ weight.

In situ hydrogel degradation. Dextran-based hydrogels formulated with degradable crosslinker (CGPQGIAGQGCR (SEQ ID NO: 3), derived from collagen I, 200 μL starting volume per gel) were incubated in PBS for 24 hours at 37° C. to assess the initial equilibrium swollen weight. The swollen hydrogels were then transferred to a 0.2 mg/ml collagenase solution in PBS and the hydrogel weight was continuously monitored over 72 hours. Control hydrogels include degradable gels incubated in PBS without collagenase and gels formulated with low, degradable crosslinker sequence (CGPQGPAGQGCR (SEQ ID NO: 4)) in the presence of 0.2 mg/mL collagenase.

Zeta potential measurements. Various polysaccharide-based solutions were prepared at a concentration of 100 mg/mL in MilliQ H₂O and approximate 800 μL solution was loaded into a disposable cuvette. The analyses were conducted at room temperature using NanoBrook ZetaPlus apparatus (Brookhaven Instruments, Holtsville, N.Y.). The zeta potential of polysaccharide solutions were measured using the electrophoretic light scattering spectrophotometer of the instrument.

Dimethylmethylene blue assay. To confirm the successful incorporation and visualization of sulfate residues in hydrogels, 200 μL hydrogels with various compositions were prepared (as described above), immersed in PBS at 37° C. for 24 hours to reach equilibrium swelling, and then incubated in a DMMB solution (16 mg dimethylmethylene blue, 3.04 g glycine, 2.37 g NaCl and 95 mL 0.1 M HCl in 1 L MilliQ H₂O, with a final solution pH approximate ˜3.0) overnight at 37° C. Hydrogels were then washed with PBS and photographed.

Cell culture and 3D encapsulation. Human dermal fibroblasts labeled with GFP (HDFs, passage 7-9) were cultured in fully supplemented fibroblast growth medium-2 (FGM-2) (Lonza). Human umbilical vein endothelial cells labeled with Ruby-LifeAct (HUVECs, passage 2-7) were cultured in fully supplemented endothelial cell growth medium-2 (EGM-2) (Lonza). For cell culture in 3D hydrogels, endothelial multicellular aggregates were fabricated using microwell culture plates (AggreWell™400, Stemcell Technologies, Vancouver, Canada) according to standard protocols and were encapsulated at ˜500 aggregates for each hydrogel composition for angiogenesis assay. For the vasculogenesis assay, GFP-HDFs and Ruby-LifeAct-HUVECs were encapsulated in the pH adjusted hydrogel precursor solutions at a final concentration of ˜9 million cells/mL (GFP-HDF: Ruby-LifeAct-HUVECs=1:2, cell density). 50 μL of the cell-containing hydrogel solution was then deposited onto uncoated glass-bottomed 35 mm dishes (MaTek Corporation, P35G-1.0-20) and allowed to polymerize for 45 minutes in 37° C. incubator before adding cell culture medium (EMG-2). HUVEC-aggregate/HUVEC-HDF co-culture hydrogels were maintained at 37° C. and 5% CO₂ in a humidified incubator with cell medium changes every two days. Cell lines were tested for mycoplasma contamination using MycoAlert Mycoplasma Detection Kit (Lonza).

Western blot. HUVECs were seeded on 2D hydrogels substrates (formulated with identical material compositions for 3D cell encapsulations) for 20-24 hours. Cells were then washed twice with ice cold PBS and lysed in RIPA buffer (1% TritonX-100, 0.1% SDS, 1% Sodium deoxycholate, 50 mM Tris-HCl, pH 7.4, 150 mM NaCl, 2 mM EDTA, and 1× protease halt (Thermo Fischer Scientific, Waltham, Mass.). Cell lysate aliquots with equal amounts of total protein (as measured using the Pierce Coomassie protein assay reagent) were separated on an SDS-PAGE gel, transferred to PVDF, blocked in 5% milk or 5% BSA (phospho-proteins) and subjected to Western blot analysis using antibodies from Cell Signaling (pVEGFR2, 2478; VEGFR2, 2479; pERK1/2, #4370; ERK1/2, #4695; pAkt, #9271; Akt, #9272; and GAPDH, #5174). The blots were developed using ECL Western blot detection reagents (Pierce), and the signal was detected on iBright™ CL1500 Imaging System (ThermoFisher Scientific, Waltham, Mass.).

Tail bleeding assay. The tail-bleeding assay was performed to determine the anti-coagulation property of heparin, Dex-MA and sulfated-Dex-MA. Briefly, an osmotic minipump (ALZET, model 1007D, Cupertino, Calif.) loaded with 100 μL of Hep-MA, Dex-MA and sulfated-Dex-MA (stock solution concentration at 100 mg/ml) was implanted under the dorsal skin 36 hours before the assay was performed. On the day of the assay, the animals were anaesthetized using an isoflurane nebulizer, which was maintained throughout the procedure. A distal 7-mm segment of the tail was amputated with a scalpel. Immediately after, the animal was placed in prone position with the tail vertically immersed in isotonic saline pre-warmed to 37° C. and the bleeding time was recorded using a timer for a maximum of 20 minutes. The animal was then euthanized by overdose of isoflurane and cervical dislocation. All animal procedures were performed at the Charles River campus animal facility, Boston University, under a protocol approved by the Institutional Animal Care and Use Committee. All experiments pertaining to this investigation conformed to the “Guide for the Care and Use of Laboratory Animals”.

In vivo angiogenesis assay and quantifications. To evaluate in vivo cell invasion and angiogenesis, hydrogels formulated with various compositions were introduced to the abdominal subcutaneous space of mouse models either through injections or implantations. Recombinant mouse growth factors, VEGF₁₆₄ and bFGF (R&D System), were incorporated during hydrogel formation at the concentrations of 18.5 nM and 5.2 nM, respectively. Mice (six-to-eight-week-old female C57BL/6NTac or BALB/c nude mice, CrTac:NCr-Foxn1nu strain, JAX or Taconic) were used in this study. The animals were anaesthetized using an isoflurane nebulizer, which was maintained throughout the procedure. For injections, the hydrogel solution (100 μL) was directly injected subcutaneously before they polymerized. For implantation, the hydrogel was pre-formed in 6 mm-diameter, 4 mm-height PDMS molds (˜60 μL in volume) before being extracted out of the mold and inserted into the subcutaneous pocket. Standard septic surgery procedures were followed by appropriate deep anesthesia using standard isoflurane throughout the procedures following by appropriate analgesic administrations. Two weeks after the injection or implantation, lysine-fixable fluorescein-conjugated dextran (FITC-dextran, 100 μL, Mw˜70 kDa, 10 mg mL⁻¹ in saline; Invitrogen) was injected retro-orbitally, five minutes post-injection, animals were euthanized by cervical dislocation under anesthesia, and hydrogel samples were harvested. All hydrogel samples were fixed in 4% paraformaldehyde (PFA) in PBS at 4° C. overnight, washed in PBS at 4° C. overnight and immersed in 30% sucrose in PBS at 4° C. for at least 2 days. Hydrogel samples were then embedded in optimum cutting temperature compound (OCT, Tissue-Tek® or Fisherbrand) in the orientation that the skin side is vertical so that the tissue cross-sections would include the skin to mark the hydrogel margin. From the middle region of each hydrogel sample, 50 μm-thick sections were collected on Superfrost Plus slides (Fisherbrand) for immunostaining and analysis.

To demonstrate blood vessels, tissue sections were stained with mouse CD31 (1:100, 4° C. overnight, clone MEC13.3, BD Pharmingen, #561814) followed by AlexaFluor 647 anti-rat antibody (1:500, RT 1 hour) and DAPI staining. To quantify invaded blood vessels and their perfusion, fluorescent images of mCD31 and FITC-dextran signals for the full 50 μm tissue section of each hydrogel sample were acquired with a Leica Microscope Objective (HCX Apo 10×/0.3W) on an upright Leica TCS SP8 multiphoton microscope with the same setting in a randomized but not overlapping fashion. The percentage (%) total area of mCD31 or FITC-dextran signal was then determined using ImageJ and the average of the fluorescent images for each hydrogel sample represents that hydrogel sample.

Fluorescent staining and microscopy. HDFs and HUVECs co-cultured in various Dex-MA hydrogels were fixed with 4% paraformaldehyde (PFA) at room temperature for 30 minutes. To visualize the organization of the actin cytoskeleton, cells were stained with phalloidin-Alexa Fluor 488, 1:1000 (Life Technologies, Carlsbad, Calif.) for overnight with nuclei counterstained with Hoechst (1:1000) for 1 hour at room temperature on next day. For immunostaining, fixed samples were first permeabilized with 0.1% Triton X-100 for 30 minutes and then blocked with 5 wt % goat serum in 0.01% Triton X-100 for 3 hour, followed by incubating with primary antibody (laminin: 1:500 rabbit polyclonal to laminin (Abcam, ab23753); CD31: 1:500 mouse monoclonal anti-CD31 I (Abcam, ab9498)) overnight and secondary antibody (1:1000 Alexa Fluor 567 goat anti-rabbit IgG (H+L) (Life Technologies) and 1:1000 Alexa Fluor 488 goat anti-mouse IgG (H+L) and) simultaneously for 1 hour at 4° C. Fluorescent images were acquired using a Leica SP8 laser scanning confocal microscope (Leica Microsystems) with a Leica HC FLUOTAR L 25×/0.95 W VISIR or a Leica HCX APO L 10×/0.30 W U-VI objective. Composite images were acquired in spatial sequence using equal laser intensity and detector gain. Unless otherwise specified, images are manually processed and presented as maximum intensity projections using ImageJ.

Statistical analysis. Statistical analysis was performed in GraphPad Prism 7, where multigroup analysis was determined by a one-way analysis of variance (ANOVA) followed by Tukey-HSD post-hoc test on all data set. Dual group analysis was performed using an unpaired Student's t-test. For experiments involving angiogenic sprouting assay, multicellularity vascular network formation experiments, and in vivo tissue vascularization characterization, results are presented in scatter plots containing mean±standard deviation, n≥4 samples were analyzed. Statistical significance is indicated by *, **, *** or **** which corresponds to P values≤0.05, 0.01, 0.001 or 0.0001, and n. s. stands for statistically insignificant.

Sequence Listing

| SEQ ID | | |
|---|---|---|
| NO | Description | Sequence |
| 1 | Adhesive RGD peptide | CGRGDS |
| 2 | Non-adhesive RGD peptide | CGRGES |
| 3 | Degradable cross-linker | CGPQGIAGQGCR |
| | derived from collagen I | |
| 4 | Slow degradable cross- | CGPQGPAGQGCR |
| | linker | |
| 5 | Cell-adhesive peptide | MNYYSNS |
| 6 | Cell-adhesive peptide | CNYYSNS |
| 7 | Cell-adhesive peptide | DAPS |
| 8 | Cell-adhesive peptide | AELDVP |
| 9 | Cell-adhesive peptide | VALDEP |
| 10 | Cell-adhesive peptide | GFOGER |
| 11 | Cell-adhesive peptide | NGRAHA |
| 12 | MMP-cleavable peptide | GPQGIAGQ |
| 13 | MMP-cleavable peptide | GPQGIWGQ |
| 14 | MMP-cleavable peptide | VPMSMRGG |
| 15 | MMP-cleavable peptide | QPQGLAK |
| 16 | MMP-cleavable peptide | GPLGLSLGK |
| 17 | MMP-cleavable peptide | GPLGMHGK |

1. A synthetic polymer comprising a polysaccharide modified to comprise one or more negatively charged functional groups,
wherein said negatively charged functional groups provide an amount of negative charge to the synthetic polymer that is sufficient to promote one or more of binding of a growth factor, growth factor activity, and vascularization; or
wherein said synthetic polymer is characterized by a zeta potential of −10 mV or less.

2. The synthetic polymer of claim 1, wherein said synthetic polymer promotes one or more of binding of growth factors, growth factor activity and vascularization to a greater extent than a corresponding polysaccharide that comprises hydroxyl groups instead of the negatively charged functional groups.

3. The synthetic polymer of claim 1, wherein said synthetic polymer does not impair blood coagulation.

4. (canceled)

5. The synthetic polymer of claim 1, wherein said growth factor is selected from the group consisting of a vascular endothelial growth factor (VEGF), a fibroblast growth factor (FGF), a bone morphogenic protein (BMP), an epidermal growth factor (EGF), a platelet derived growth factor (PDGF), a WNT, and a combination thereof.

6. The synthetic polymer of claim 1, wherein said growth factor[s] is a cytokine, optionally wherein the cytokine is an interleukin, an interferon, or chemokine.

7-8. (canceled)

9. The synthetic polymer of claim 1, wherein said synthetic polymer promotes an equivalent or greater amount of growth factor dependent cell signaling as heparin.

10-13. (canceled)

14. The synthetic polymer of claim 1, wherein said synthetic polymer is characterized by a zeta potential of about −10 mV to about −60 mV.

15. (canceled)

16. The synthetic polymer of claim 1, wherein said synthetic polymer comprises an average of 0.1 to 2.0 functional groups per monosaccharide unit.

17-18. (canceled)

19. The synthetic polymer of claim 1, wherein said polysaccharide is derived from a naturally occurring polysaccharide.

20. The synthetic polymer of claim 19, wherein said naturally occurring polysaccharide is selected from the group consisting of alginate, agarose, chondroitin sulfate, chitin/chitosan, cellulose, dextran, starch, glycogen, galactogen, inulin, pectin, and hyaluronic acid.

21-23. (canceled)

24. The synthetic polymer of claim 20, wherein said polysaccharide is dextran.

25. The synthetic polymer of claim 1, wherein said negatively charged functional groups are selected from the group consisting of a sulfate group, a phosphate group, a carboxyl group and combinations thereof.

26. The synthetic polymer of claim 1, wherein said synthetic polymer has a mean weight-average molecular weight of about 5 kDa to about 650 kDa.

27-31. (canceled)

32. A synthetic dextran polymer having one or more negatively charged functional groups, wherein said synthetic dextran polymer has a zeta potential of −10 mV or less.

33. The synthetic dextran polymer of claim 32, wherein the synthetic dextran polymer comprises an average of at least 0.5 negatively charged functional groups per monosaccharide unit of the polymer.

34-35. (canceled)

36. A method for generating the synthetic polymer of claim 1, said method comprising contacting a polysaccharide comprising hydroxyl groups with a moiety comprising a negatively charged functional group under conditions that allow for conversion of one or more of said hydroxyl groups into negatively charged functional groups.

37. A hydrogel comprising a plurality of the synthetic polymers of claim 1, wherein the synthetic polymers are cross-linked to each other by a cross-linker.

38. (canceled)

39. The hydrogel of claim 37, wherein said polysaccharide is dextran.

40-44. (canceled)

45. The hydrogel of claim 37, wherein said cross-linker is a cleavable cross-linker.

46-49. (canceled)

50. The hydrogel of claim 37, further comprising a cell-adhesive peptide.

51-52. (canceled)

53. The hydrogel of claim 37, further comprising at least one growth factor.

54-55. (canceled)

56. The hydrogel of claim 37, further comprising a population of cells.

57. (canceled)

58. The hydrogel of claim 56, wherein said population of cells comprises two or more cell types.

59. The hydrogel of claim 56, wherein said population of cells comprises parenchymal cells, stromal cells, or both parenchymal and stromal cells.

60. The hydrogel of claim 59, wherein said parenchymal cells are of heart, lung, liver, kidney, adrenal gland, pituitary gland, pancreas, or muscle.

61-62. (canceled)

63. The hydrogel of claim 59, wherein said stromal cells comprise endothelial cells, fibroblasts, or endothelial cells and fibroblasts.

64. A composition comprising the synthetic polymer of claim 1.

65. (canceled)

66. A method of promoting vascularization of a cell implant or an engineered tissue construct in a subject, comprising administering to the subject the cell implant or engineered tissue construct in combination with the synthetic polymer of claim 1.

67. (canceled)

68. A method of promoting cell survival in a cell implant or an engineered tissue construct in a subject, comprising administering to the subject the cell implant or engineered tissue construct in combination with the synthetic polymer of claim 1.

69. (canceled)

70. A method of promoting engraftment of a cell implant or an engineered tissue construct in a subject, comprising administering to the subject the cell implant or engineered tissue construct in combination with the synthetic polymer of claim 1.

71. (canceled)

72. A method of promoting vascularization in a diseased or damaged tissue in a subject, comprising administering to the subject the synthetic polymer of claim 1.

73-74. (canceled)

75. A method of promoting vascularization of a tissue graft in a subject, comprising contacting a tissue to be grafted with the synthetic polymer of claim 1 prior to grafting of the tissue for a sufficient time to promote vascularization of the tissue graft upon grafting in the subject.

76. A method of promoting a growth factor-dependent cell therapy, comprising administering to a subject the growth factor-dependent cell therapy in combination with the synthetic polymer of claim 1.

77. (canceled)
